SEQUENCE LISTING (161 sequences)

SEQ ID NO: 1 - protein, 299 aa, Mus musculus
SKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 2 - protein, 293 aa, Mus musculus
SSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 3 - protein, 280 aa, Mus musculus
GTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 4 - protein, 103 aa, Mus musculus
MADKILRAKRKQFINSVSIGTINGLLDELLEKRVLNQEEMDKIKLANITAMDKARDLCDHVSKKGPQASQIFITYICNEDCYLAGILELQSAPSAETFVATED

SEQ ID NO: 5 - protein, 402 aa, Mus musculus
MADKILRAKRKQFINSVSIGTINGLLDELLEKRVLNQEEMDKIKLANITAMDKARDLCDHVSKKGPQASQIFITYICNEDCYLAGILELQSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 6 - protein, 285 aa, Homo sapiens
NPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 7 - protein, 115 aa, Homo sapiens
MADKVLKEKRKLFIRSMGDGLLDELLQTRVLNKEEMEKVKRENATVMDKTRALIDSVIPKGAQACQICITYICEEDSYLAGTLGLSADQTSGNYLNMQDSQGVLSSFPAPQAVQD

SEQ ID NO: 8 - protein, 400 aa, Homo sapiens
MADKVLKEKRKLFIRSMGDGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 9 - protein, 273 aa, Homo sapiens
ALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 10 - protein, 104 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTD

SEQ ID NO: 11 - protein, 377 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 12 - protein, 317 aa, Homo sapiens
SLRKNRVAHQMFTQTLLNMDQKITSVKPLLQIEAGPPESAESTNILKLCPREEFLRLCKKNHDEIYPIKKREDRRRLALIICNTKFDHLPARNGAHYDIVGMKRLLQGLGYTVVDEKNLTARDMESVLRAFAARPEHKSSDSTFLVLMSHGILEGICGTAHKKKKPDVLLYDTIFQIFNNRNCLSLKDKPKVIIVQACRGEKHGELWVRDSPASLALISSQSSENLEADSVCKIHEEKDFIAFCSSTPHNVSWRDRTRGSIFITELITCFQKYSCCCHLMEIFRKVQKSFEVPQAKAQMPTIERATLTRDFYLFPGN

SEQ ID NO: 13 - protein, 117 aa, Homo sapiens
MAEDSGKKKRRKNFEAMFKGILQSGLDNFVINHMLKNNVAGQTSIQTLVPNTDQKSTSVKKDNHKKKTVKMLEYLGKDVLHGVFNYLAKHDVLTLKEEEKKKYYDTKIEDKALILVD

SEQ ID NO: 14 - protein, 434 aa, Homo sapiens
MAEDSGKKKRRKNFEAMFKGILQSGLDNFVINHMLKNNVAGQTSIQTLVPNTDQKSTSVKKDNHKKKTVKMLEYLGKDVLHGVFNYLAKHDVLTLKEEEKKKYYDTKIEDKALILVDSLRKNRVAHQMFTQTLLNMDQKITSVKPLLQIEAGPPESAESTNILKLCPREEFLRLCKKNHDEIYPIKKREDRRRLALIICNTKFDHLPARNGAHYDIVGMKRLLQGLGYTVVDEKNLTARDMESVLRAFAARPEHKSSDSTFLVLMSHGILEGICGTAHKKKKPDVLLYDTIFQIFNNRNCLSLKDKPKVIIVQACRGEKHGELWVRDSPASLALISSQSSENLEADSVCKIHEEKDFIAFCSSTPHNVSWRDRTRGSIFITELITCFQKYSCCCHLMEIFRKVQKSFEVPQAKAQMPTIERATLTRDFYLFPGN

SEQ ID NO: 15 - protein, 267 aa, Homo sapiens
REQDSESQTLDKVYQMKSKPRGYCLIINNHNFAKAREKVPKLHSIRDRNGTHLDAGALTTTFEELHFEIKPHHDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 16 - protein, 212 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSP

SEQ ID NO: 17 - protein, 479 aa, Homo sapiens [sequence omitted]

SEQ ID NO: 18 - protein, 277 aa, Homo sapiens
GALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 19 - protein, 139 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDV

SEQ ID NO: 20 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 21 - protein, 289 aa, Mus musculus
MSSVKLWPTGASAVPLVSREELKKLEFVGKGGFGVVFRAHHRTWNHDVAVKIVNSKKISWEVKAMVNLRNENVLLLLGVTEDLQWDFVSGQALVTRFMENGSLAGLLQPECPRPWPLLCRLLQEVVLGMCYLHSLNPPLLHRDLKPSNILLDPELHAKLADFGLSTFQGGSQSGSGSGSGSRDSGGTLAYLDPELLFDVNLKASKASDVYSFGILVWAVLAGREAELVDKTSLIRETVCDRQSRPPLTELPPGSPETPGLEKLKELMIHCWGSQSENRPSFQDCEPKTN

SEQ ID NO: 22 - protein, 23 aa, Mus musculus
NNCSEVQIGNYNSLVAPPRTTAS

SEQ ID NO: 23 - protein, 486 aa, Mus musculus
MSSVKLWPTGASAVPLVSREELKKLEFVGKGGFGVVFRAHHRTWNHDVAVKIVNSKKISWEVKAMVNLRNENVLLLLGVTEDLQWDFVSGQALVTRFMENGSLAGLLQPECPRPWPLLCRLLQEVVLGMCYLHSLNPPLLHRDLKPSNILLDPELHAKLADFGLSTFQGGSQSGSGSGSGSRDSGGTLAYLDPELLFDVNLKASKASDVYSFGILVWAVLAGREAELVDKTSLIRETVCDRQSRPPLTELPPGSPETPGLEKLKELMIHCWGSQSENRPSFQDCEPKTNEVYNLVKDKVDAAVSEVKHYLSQHRSSGRNLSAREPSQRGTEMDCPRETMVSKMLDRLHLEEPSGPVPGKCPERQAQDTSVGPATPARTSSDPVAGTPQIPHTLPFRGTTPGPVFTETPGPHPQRNQGDGRHGTPWYPWTPPNPMTGPPALVFNNCSEVQIGNYNSLVAPPRTTASSSAKYDQAQFGRGRGWQPFHK

SEQ ID NO: 24 - protein, 430 aa, Mus musculus
MSSVKLWPTGASAVPLVSREELKKLEFVGKGGFGVVFRAHHRTWNHDVAVKIVNSKKISWEVKAMVNLRNENVLLLLGVTEDLQWDFVSGQALVTRFMENGSLAGLLQPECPRPWPLLCRLLQEVVLGMCYLHSLNPPLLHRDLKPSNILLDPELHAKLADFGLSTFQGGSQSGSGSGSGSRDSGGTLAYLDPELLFDVNLKASKASDVYSFGILVWAVLAGREAELVDKTSLIRETVCDRQSRPPLTELPPGSPETPGLEKLKELMIHCWGSQSENRPSFQDCEPKTNEVYNLVKDKVDAAVSEVKHYLSQHRSSGRNLSAREPSQRGTEMDCPRETMVSKMLDRLHLEEPSGPVPGKCPERQAQDTSVGPATPARTSSDPVAGTPQIPHTLPFRGTTPGPVFTETPGPHPQRNQGDGRHGTPWYPWTP

SEQ ID NO: 25 - protein, 289 aa, Homo sapiens
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQM

SEQ ID NO: 26 - protein, 23 aa, Homo sapiens
NIYNCSGVQVGDNNYLTMQQTTA

SEQ ID NO: 27 - protein, 518 aa, Homo sapiens
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGK

SEQ ID NO: 28 - protein, 233 aa, Homo sapiens
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAIC

SEQ ID NO: 29 - protein, 149 aa, Homo sapiens
PPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 30 - protein, 671 aa, Homo sapiens
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 31 - protein, 299 aa, Homo sapiens
MNGEAICSALPTIPYHKLADLRYLSRGASGTVSSARHADWRVQVAVKHLHIHTPLLDSERKDVLREAEILHKARFSYILPILGICNEPEFLGIVTEYMPNGSLNELLHRKTEYPDVAWPLRFRILHEIALGVNYLHNMTPPLLHHDLKTQNILLDNEFHVKIADFGLSKWRMMSLSQSRSSKSAPEGGTIIYMPPENYEPGQKSRASIKHDIYSYAVITWEVLSRKQPFEDVTNPLQIMYSVSQGHRPVINEESLPYDIPHRARMISLIESGWAQNPDERPSFLKCLIELEPVLRTFEE

SEQ ID NO: 32 - protein, 115 aa, Homo sapiens
GNSERLQPGIAQQWIQSKREDIVNQMTEACLNQSLDALLSRDLIMKEDYELVSTKPTRTSKVRQLLDTTDIQGEEFAKVIVQKLKDNKQMGLQPYPEILVVSRSPSLNLLQNKSM

SEQ ID NO: 33 - protein, 540 aa, Homo sapiens
MNGEAICSALPTIPYHKLADLRYLSRGASGTVSSARHADWRVQVAVKHLHIHTPLLDSERKDVLREAEILHKARFSYILPILGICNEPEFLGIVTEYMPNGSLNELLHRKTEYPDVAWPLRFRILHEIALGVNYLHNMTPPLLHHDLKTQNILLDNEFHVKIADFGLSKWRMMSLSQSRSSKSAPEGGTIIYMPPENYEPGQKSRASIKHDIYSYAVITWEVLSRKQPFEDVTNPLQIMYSVSQGHRPVINEESLPYDIPHRARMISLIESGWAQNPDERPSFLKCLIELEPVLRTFEEITFLEAVIQLKKTKLQSVSSAIHLCDKKKMELSLNIPVNHGPQEESCGSSQLHENSGSPETSRSLPAPQDNDFLSRKAQDCYFMKLHHCPGNHSWDSTISGSQRAAFCDHKTTPCSSAIINPLSTAGNSERLQPGIAQQWIQSKREDIVNQMTEACLNQSLDALLSRDLIMKEDYELVSTKPTRTSKVRQLLDTTDIQGEEFAKVIVQKLKDNKQMGLQPYPEILVVSRSPSLNLLQNKSM

SEQ ID NO: 34 - protein, 141 aa, Homo sapiens
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDA

SEQ ID NO: 35 - protein, 330 aa, Homo sapiens
DEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK

SEQ ID NO: 36 - protein, 471 aa, Homo sapiens
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK

SEQ ID NO: 37 - protein, 275 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTD

SEQ ID NO: 38 - protein, 209 aa, Homo sapiens
GVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 39 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 40 - protein, 77 aa, Artificial sequence
  Synthetic
MGTRTEIIRELERSLREQEELAKRLKELLRELERLQREGSSDEDVRELLREIKELVEEIEKLAREQKYLVEELKRQD

SEQ ID NO: 41 - protein, 77 aa, Artificial sequence; identical to SEQ ID NO: 40
  Synthetic

SEQ ID NO: 42 - protein, 77 aa, Artificial sequence; identical to SEQ ID NO: 40
  Synthetic

SEQ ID NO: 43 - protein, 99 aa, Artificial sequence
  Synthetic
GSGSGSGSHMSEELRAVADLQRLNIELARKLLEAVARLQELNIDLVRKTSELTDEKTIREEIRKVKEESKRIVEEAEEEIRRAKEESRYIADESRGSLE

SEQ ID NO: 44 - protein, 73 aa, Artificial sequence
  Synthetic
MTEDEELQRVEEENRELERKAKELEYKIEEIRKKVNGRSPQAEALLMQALALLLIAEALSSQAKALLMIARST

SEQ ID NO: 45 - protein, 492 aa, Artificial sequence
  Synthetic
MGTRTEIIRELERSLREQEELAKRLKELLRELERLQREGSSDEDVRELLREIKELVEEIEKLAREQKYLVEELKRQDGSEGSEGSEGSRTMADKILRAKRKQFINSVSIGTINGLLDELLEKRVLNQEEMDKIKLANITAMDKARDLCDHVSKKGPQASQIFITYICNEDCYLAGILELQSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 46 - protein, 383 aa, Artificial sequence
  Synthetic
MGTRTEIIRELERSLREQEELAKRLKELLRELERLQREGSSDEDVRELLREIKELVEEIEKLAREQKYLVEELKRQDGSEGSEGSEGSRTSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 47 - protein, 366 aa, Artificial sequence
  Synthetic
MTEDEELQRVEEENRELERKAKELEYKIEEIRKKVNGRSPQAEALLMQALALLLIAEALSSQAKALLMIARSTGSEGSEGSEGSRTGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 48 - protein, 569 aa, Artificial sequence
  Synthetic
MGTRTEIIRELERSLREQEELAKRLKELLRELERLQREGSSDEDVRELLREIKELVEEIEKLAREQKYLVEELKRQDGSEGSEGSEGSRTMDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHHDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 49 - protein, 369 aa, Artificial sequence
  Synthetic
MGTRTEIIRELERSLREQEELAKRLKELLRELERLQREGSSDEDVRELLREIKELVEEIEKLAREQKYLVEELKRQDGSGSGSTSGGGGSRGSHMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSDV

SEQ ID NO: 50 - protein, 504 aa, Artificial sequence
  Synthetic
MGTRTEIIRELERSLREQEELAKRLKELLRELERLQREGSSDEDVRELLREIKELVEEIEKLAREQKYLVEELKRQDGSEGSEGSEGSMDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 51 - protein, 369 aa, Artificial sequence
  Synthetic
MGTRTEIIRELERSLREQEELAKRLKELLRELERLQREGSSDEDVRELLREIKELVEEIEKLAREQKYLVEELKRQDGSEGSEGSEGSRTGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVD

SEQ ID NO: 52 - protein, 585 aa, Artificial sequence
  Synthetic
MSSVKLWPTGASAVPLVSREELKKLEFVGKGGFGVVFRAHHRTWNHDVAVKIVNSKKISWEVKAMVNLRNENVLLLLGVTEDLQWDFVSGQALVTRFMENGSLAGLLQPECPRPWPLLCRLLQEVVLGMCYLHSLNPPLLHRDLKPSNILLDPELHAKLADFGLSTFQGGSQSGSGSGSGSRDSGGTLAYLDPELLFDVNLKASKASDVYSFGILVWAVLAGREAELVDKTSLIRETVCDRQSRPPLTELPPGSPETPGLEKLKELMIHCWGSQSENRPSFQDCEPKTNEVYNLVKDKVDAAVSEVKHYLSQHRSSGRNLSAREPSQRGTEMDCPRETMVSKMLDRLHLEEPSGPVPGKCPERQAQDTSVGPATPARTSSDPVAGTPQIPHTLPFRGTTPGPVFTETPGPHPQRNQGDGRHGTPWYPWTPPNPMTGPPALVFNNCSEVQIGNYNSLVAPPRTTASSSAKYDQAQFGRGRGWQPFHKGSGSGSGSHMSEELRAVADLQRLNIELARKLLEAVARLQELNIDLVRKTSELTDEKTIREEIRKVKEESKRIVEEAEEEIRRAKEESRYIADESRGSLE

SEQ ID NO: 53 - protein, 529 aa, Artificial sequence
  Synthetic
MSSVKLWPTGASAVPLVSREELKKLEFVGKGGFGVVFRAHHRTWNHDVAVKIVNSKKISWEVKAMVNLRNENVLLLLGVTEDLQWDFVSGQALVTRFMENGSLAGLLQPECPRPWPLLCRLLQEVVLGMCYLHSLNPPLLHRDLKPSNILLDPELHAKLADFGLSTFQGGSQSGSGSGSGSRDSGGTLAYLDPELLFDVNLKASKASDVYSFGILVWAVLAGREAELVDKTSLIRETVCDRQSRPPLTELPPGSPETPGLEKLKELMIHCWGSQSENRPSFQDCEPKTNEVYNLVKDKVDAAVSEVKHYLSQHRSSGRNLSAREPSQRGTEMDCPRETMVSKMLDRLHLEEPSGPVPGKCPERQAQDTSVGPATPARTSSDPVAGTPQIPHTLPFRGTTPGPVFTETPGPHPQRNQGDGRHGTPWYPWTPGSGSGSGSHMSEELRAVADLQRLNIELARKLLEAVARLQELNIDLVRKTSELTDEKTIREEIRKVKEESKRIVEEAEEEIRRAKEESRYIADESRGSLE

SEQ ID NO: 54 - protein, 570 aa, Artificial sequence
  Synthetic
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSKGSGSGSGSHMSEELRAVADLQRLNIELARKLLEAVARLQELNIDLVRKTSELTDEKTIREEIRKVKEESKRIVEEAEEEIRRAKEESRYIADESRGSLE

SEQ ID NO: 55 - protein, 240 aa, Artificial sequence
  Synthetic
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDAGSGSGSGSHMSEELRAVADLQRLNIELARKLLEAVARLQELNIDLVRKTSELTDEKTIREEIRKVKEESKRIVEEAEEEIRRAKEESRYIADESRGSLE

SEQ ID NO: 56 - protein, 570 aa, Artificial sequence
  Synthetic
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPHMTEDEELQRVEEENRELERKAKELEYKIEEIRKKVNGRSPQAEALLMQALALLLIAEALSSQAKALLMIARSTGSEGSEGSEGSRT

SEQ ID NO: 57 - protein, 361 aa, Artificial sequence
  Synthetic
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDMTEDEELQRVEEENRELERKAKELEYKIEEIRKKVNGRSPQAEALLMQALALLLIAEALSSQAKALLMIARSTGSEGSEGSEGSRT

SEQ ID NO: 58 - protein, 75 aa, Artificial sequence
  Synthetic
TRTRSLREQEEIIRELERSLREQEELLRELERLQREGSSDEDVRELLREIKKLAREQKYLVEELKKLAREQKRQD

SEQ ID NO: 59 - protein, 75 aa, Artificial sequence
  Synthetic
TRTEIIRELERSLREQERSLREQEELLRELERLQREGSSDEDVRELLREIKKLAREQKKLAREQKYLVEELKRQD

SEQ ID NO: 60 - protein, 75 aa, Artificial sequence
  Synthetic
TRTEIIRELERSLREQEELAKRLKRSLREQERLQREGSSDEDVRKLAREQKELVEEIEKLAREQKYLVEELKRQD

SEQ ID NO: 61 - protein, 75 aa, Artificial sequence
  Synthetic
TRTEIIRELEELAKRLKRSLREQERSLREQERLQREGSSDEDVRKLAREQKKLAREQKELVEEIEYLVEELKRQD

SEQ ID NO: 62 - protein, 76 aa, Artificial sequence
  Synthetic
  MISC_FEATURE (1)..(1): optionally absent
GTAIEANSRMLKALIEIAKAIWKALWANSLLLEATSRGDTERMRQWAEEAREIYKEAKKIIDEADEIVKEAKERHD

SEQ ID NO: 63 - protein, 87 aa, Artificial sequence
  Synthetic
  MISC_FEATURE (86)..(87): optionally absent
SEELRAVADLQRLNIELARKLLEAVARLQELNIDLVRKTSELTDEKTIREEIRKVKEESKRIVEEAEEEIRRAKEESRYIADESRGS

SEQ ID NO: 64 - protein, 77 aa, Artificial sequence
  Synthetic
  MISC_FEATURE (1)..(1): optionally absent
GTSDYIIEQIQRDQEEARKKVEEAEERLERVKEASKRGVSSDQLLDLIRELAEIIEELIRIIRRSNEAIKELIKNQS

SEQ ID NO: 65 - protein, 77 aa, Artificial sequence
  Synthetic
  MISC_FEATURE (1)..(1): optionally absent
GSEDYKLREAQRELDKQRKDTEEIRKRLKEIQRLTDERTSTADELIKELREIIRRLQEQSEKLREIIEELEKIIRKR

SEQ ID NO: 66 - protein, 76 aa, Artificial sequence
  Synthetic
  MISC_FEATURE (1)..(1): optionally absent
GTRTEIIRELERSLREQEELAKRLKELLRELERLQREGSSDEDVRELLREIKELVEEIEKLAREQKYLVEELKRQD

SEQ ID NO: 67 - protein, 77 aa, Artificial sequence
  Synthetic
  MISC_FEATURE (1)..(1): optionally absent
GTDTDELLRLAKEQAELLKEIKKLVEEIARLVKEIQEDPSDELLKTLAELVRKLKELVEDMERSMKEQLYIIKKQKS

SEQ ID NO: 68 - protein, 71 aa, Artificial sequence
  Synthetic
  MISC_FEATURE (1)..(1): optionally absent
GSKDTEDSRKIWEDIRRLLEEARKNSEEIWKEITKNPDTSEIARLLSEQLLEIAEMLVRIAELLSRQTEQR

SEQ ID NO: 69 - protein, 75 aa, Artificial sequence
  Synthetic
  MISC_FEATURE (1)..(1): optionally absent
GTKYEIREALKEAQKQLEDLKRMLDELRRNLEELKRNPSEDALVENNELIVRVLEVIVENNRSIIEILKLLAKSD

SEQ ID NO: 70 - protein, 75 aa, Artificial sequence
  Synthetic
  MISC_FEATURE (1)..(1): optionally absent
GTKYKIREMLEEAKRSLEELRRILEKLKESLRELRRNPSEDALVNNNEVIVKAIEASVENQRIIIELARMLAESD

SEQ ID NO: 71 - protein, 75 aa, Artificial sequence
  Synthetic
  MISC_FEATURE (1)..(1): optionally absent
GTKYRIKDTLRELKRALEELKKILEELQRSLEELRRNPSEDALVNNNEVIVKAIEAAVRAIEISAENQRMLAESD

SEQ ID NO: 72 - protein, 75 aa, Artificial sequence
  Synthetic
  MISC_FEATURE (1)..(1): optionally absent
GTKYEARKQLEEMKKQLKDLKRSLERLREILERLEENPSEDVIVEAIRAIVENNKQIVENNRSIIENNETIIRSD

SEQ ID NO: 73 - protein, 75 aa, Artificial sequence
  Synthetic
  MISC_FEATURE (1)..(1): optionally absent
GTKYELRRQLEELEKLLRELRKSLDELRKILEELERNPSEDVIVRAIKASVKNQEIIVEVLRAIIENNKTIAKSD

SEQ ID NO: 74 - protein, 75 aa, Artificial sequence
  Synthetic
  MISC_FEATURE (1)..(1): optionally absent
GTKYELRRALEELEKALQELREMLRKLKESLEELKKNPSEDALVRNNELIVEVLRVIVEVLSIIARVLEINARSD

SEQ ID NO: 75 - protein, 75 aa, Artificial sequence
  Synthetic
  MISC_FEATURE (1)..(1): optionally absent
GTKYELRRALEELEKALRELKKSLDELERSLEELEKNPSEDALVENNRLNVENNKIIVEVLRIIAEVLKINAKSD

SEQ ID NO: 76 - protein, 75 aa, Artificial sequence
  Synthetic
  MISC_FEATURE (1)..(1): optionally absent
GTKYKIKETLKRLEDSLRELRRILEELKEMLERLEKNPDKDVIVEVLKVIVKAIEASVENQRISAENQKALAESD

SEQ ID NO: 77 - protein, 75 aa, Artificial sequence
  Synthetic
  MISC_FEATURE (1)..(1): optionally absent
GTKYEIKKALKELEEAIQKLKKSLKELKESLKELQKNPSEDALVKNNSLNVANNEIIVEVLEIIARILELLARSD

SEQ ID NO: 78 - protein, 75 aa, Artificial sequence
  Synthetic
  MISC_FEATURE (1)..(1): optionally absent
GTKYEIKEALRELNRALKELKEALRELERSLRELQKNPDKDALVRNNELNVDVARIIVEVLSIIARVLELLAKSD

SEQ ID NO: 79 - protein, 75 aa, Artificial sequence; identical to SEQ ID NO: 74
  Synthetic
  MISC_FEATURE (1)..(1): optionally absent

SEQ ID NO: 80 - protein, 75 aa, Artificial sequence; identical to SEQ ID NO: 75
  Synthetic
  MISC_FEATURE (1)..(1): optionally absent

SEQ ID NO: 81 - protein, 75 aa, Artificial sequence
  Synthetic
  MISC_FEATURE (1)..(1): optionally absent
GTKYELREAIRKLEEALRKLKKALDELRKSLEELKKNPSEDALVRNNELNVKVAEIIVKVLKIIAEAIKINAKSD

SEQ ID NO: 82 - protein, 74 aa, Artificial sequence
  Synthetic
  MISC_FEATURE (1)..(1): optionally absent
GTEEYKLRELLKRHNEVLKELQKAAKEAEEVAERFKKTNDITEAIRVIADLLRAIVKAIETNSRVVKMIVELNE

SEQ ID NO: 83 - protein, 75 aa, Artificial sequence
  Synthetic
  MISC_FEATURE (1)..(1): optionally absent
GTKYIEKLLREAQRTLEELKRLLEELKEMLKELERANATDARLIAEVIRVIVEVLRASVENQEMIIRILKAITEE

SEQ ID NO: 84 - protein, 76 aa, Artificial sequence
  Synthetic
  MISC_FEATURE (75)..(76): optionally absent
TEKDVLRIIVKNNEIIVKVLSVIAEVLKIIAKILENPSEYMLKELKKALKELEKMLKELRKSLKELKEALRELEGS

SEQ ID NO: 85 - protein, 75 aa, Artificial sequence
  Synthetic
  MISC_FEATURE (1)..(1): optionally absent
GTLDYKLDEMLKKLEKSREEMEKMAQELRRALEELEKNSNVDKVLKIIIKAIQLSIENQKLNLEAVRLLIEAQKS

SEQ ID NO: 86 - protein, 75 aa, Artificial sequence; identical to SEQ ID NO: 76
  Synthetic
  MISC_FEATURE (1)..(1): optionally absent

SEQ ID NO: 87 - protein, 77 aa, Artificial sequence
  Synthetic
  MISC_FEATURE (1)..(1): optionally absent
GTDEYKWKEEVRRFEEEAKKWEEELKEMRKRIEDAKKGRPTLKVNLEAAEALLEAARLIVEAAKLLLAAAKLNEKQN

SEQ ID NO: 88 - protein, 77 aa, Artificial sequence
  Synthetic
  MISC_FEATURE (1)..(1): optionally absent
GSDEDRKAKELIERQRKLTDEAEEWAKQNEEIAKKIEKQPDTSLVARMLANVSRMLLATNRALLANTEALEALIRKT

SEQ ID NO: 89 - protein, 76 aa, Artificial sequence; identical to SEQ ID NO: 62
  Synthetic
  MISC_FEATURE (1)..(1): optionally absent

SEQ ID NO: 90 - protein, 135 aa, Artificial sequence
  Synthetic
  MISC_FEATURE (134)..(135): optionally absent
SALEKIAKLIIEAARLSAELARRAARASAEMARKAIEAVSEERGSESLLKIVADLIVESQEAVVRLIIESQQIAAKLAEDLIRAAKEAASDESKMEEVAKEVQERAERAARDIERKLKRVLEELDYKLKESRDGS

SEQ ID NO: 91 - protein, 135 aa, Artificial sequence
  Synthetic
  MISC_FEATURE (134)..(135): optionally absent
TALEIAVRLNREAAREAARENADTARKAARRIAEVAKRLAEENRDAKLAARLLAEIARLLAELIARQSELLAEWLATQSKLAAELARKDTSATDEAERIRKESEELLDKVREEIKRLEDEVSKTIEELSERVRGS

SEQ ID NO: 92 - protein, 135 aa, Artificial sequence
  Synthetic
  MISC_FEATURE (134)..(135): optionally absent
SILELAHESNRRALEMASRANREAMKAAREMIRAASEAARRAGSSNDKDSLRMIEEALRLALRMIEETNKKAVRMVLENNRKMVEAEKKKLSEEEIKRIAKETEDRMREIARRASEEARRLAEEIKREADYRSGS

SEQ ID NO: 93 - protein, 86 aa, Artificial sequence
  Synthetic
  MISC_FEATURE (85)..(86): optionally absent
EELRAVADLQRLNIELARKLLEAVARLQELNIDLVRKTSELTDEKTIREEIRKVKEESKRIVEEAEEEIRRAKEESRYIADESRGS

SEQ ID NO: 94 - protein, 85 aa, Artificial sequence
  Synthetic
  MISC_FEATURE (1)..(1): optionally absent
GTERKDRLRKELKRIAEETDKWVEELKEELERILRTIEELRKDPSSEVIVDIARIQLEALREVIRVVAENSKAILEAIHRVIEEG

SEQ ID NO: 95 - protein, 92 aa, Artificial sequence
  Synthetic
  MISC_FEATURE (91)..(92): optionally absent
SKEVRLQKLNAEIMKEIMELIIRLQEANARIIEELVRLIIDLERSTDSKRMIEEIRKVAERAIEESKRLLEEAEKAMRRAIYESEDALREGS

SEQ ID NO: 96 - protein, 87 aa, Artificial sequence
  Synthetic
  MISC_FEATURE (1)..(1): optionally absent
GSKVEELLRKSEEAAERAKRELERLLEESERIVAEAQALAEKYESQKVWVRILIELIRATNRMLAEIARILLEMIEVTNRMIAESTK

SEQ ID NO: 97 - protein, 92 aa, Artificial sequence
  Synthetic
  MISC_FEATURE (91)..(92): optionally absent
SEQLKEIARILIKLIESLTRFILEVARILIELIEETQRLIVASTDSDESELERIARESKKKAKKALDELKKIVDDQRREAKKAIEELEYDGS

SEQ ID NO: 98 - protein, 71 aa, Artificial sequence; identical to SEQ ID NO: 68
  Synthetic
  MISC_FEATURE (1)..(1): optionally absent

SEQ ID NO: 99 - protein, 76 aa, Artificial sequence
  Synthetic
  MISC_FEATURE (1)..(1): optionally absent
GTAYELLRKAEELEKKQQELLKRQEELAKTAEELRKKGGNADSMMKIIKESTRIVRESTEIVKELLKIIRELRRQS

SEQ ID NO: 100 - protein, 77 aa, Artificial sequence
  Synthetic
  MISC_FEATURE (1)..(1): optionally absent
GTRTEYLKKLAEEAKELAKRSRELSKESRRLSEEARRDPDKEKLLRVVKKLQEVIEELQRVIEELLRVIKEALENQS

SEQ ID NO: 101 - protein, 77 aa, Artificial sequence
  Synthetic
  MISC_FEATURE (1)..(1): optionally absent
GTETEYQRELAREARRLAKRSRELSERSRKLSEDAKRDPDKDKLLEVVERLQQVIEELQKVIEELLRVIESSLKTIS

SEQ ID NO: 102 - protein, 77 aa, Artificial sequence; identical to SEQ ID NO: 64
  Synthetic
  MISC_FEATURE (1)..(1): optionally absent

SEQ ID NO: 103 - protein, 77 aa, Artificial sequence
  Synthetic
  MISC_FEATURE (1)..(1): optionally absent
GTEEYRRKEQEERTKEQQERTERQRRKTEELKRATKEGTLTPEEAIRQAQKQSENAERQSREAEKQSREANEALRKR

SEQ ID NO: 104 - protein, 77 aa, Artificial sequence; identical to SEQ ID NO: 65
  Synthetic
  MISC_FEATURE (1)..(1): optionally absent

SEQ ID NO: 105 - protein, 77 aa, Artificial sequence
  Synthetic
  MISC_FEATURE (1)..(1): optionally absent
GSEDYKLKELQKRNKKQEEEAKRNDDERKKIEELTRKRTSTADELIRELQRSNEEMQRSQREMQDQSRRLEDIIRKR

SEQ ID NO: 106 - protein, 77 aa, Artificial sequence
  Synthetic
  MISC_FEATURE (1)..(1): optionally absent
GTEDYKRREAERKLQKQQEELKELKRKLEEIRELHEKGVGSPDRLIRELERIIRELQRMQKENEKIIKELQRIIKKR

SEQ ID NO: 107 - protein, 85 aa, Artificial sequence
  Synthetic
  MISC_FEATURE (1)..(1): optionally absent
GTESKYLLEEARRLKDEARKLKEEAKKVKEESRKLIERIDRGEDSDRELLERLKEQNNRLLEIIERLLEIIERLLKLIEEWTRDS

SEQ ID NO: 108 - protein, 85 aa, Artificial sequence
  Synthetic
  MISC_FEATURE (1)..(1): optionally absent
GTEEDYAEREIRKMKEEQKRQRKRLEELERELQEMQEKKREGTSDAKEVIDQLERIIRELQEIIRSQEDITRKLEEIIRRMKENS

SEQ ID NO: 109 - protein, 77 aa, Artificial sequence
  Synthetic
  MISC_FEATURE (1)..(1): optionally absent
GTNKEELKRTMEEQQRILEKLLRTIKEQKEILRKQEEGRATKEELKRLTKLAQEQERMMRELIDLARKQAYLLKRES

SEQ ID NO: 110 - protein, 77 aa, Artificial sequence
  Synthetic
  MISC_FEATURE (1)..(1): optionally absent
GTREEKIRRILEEIQKIMEEIKRIMEEIKRTQEEAEKHGSSKKAIEKQKELLRRLEELLRKLERLLRELEYLMRDEK

SEQ ID NO: 111 - protein, 77 aa, Artificial sequence
  Synthetic
  MISC_FEATURE (1)..(1): optionally absent
GTREEWLYRILELIERIERLIKEIIRLSRRALELLENNASNEEWAQEIKEMQRKIQEWLKQILEWLKKIKEWIRESQ

SEQ ID NO: 112 - protein, 76 aa, Artificial sequence; identical to SEQ ID NO: 66
  Synthetic
  MISC_FEATURE (1)..(1): optionally absent

SEQ ID NO: 113 - protein, 77 aa, Artificial sequence; identical to SEQ ID NO: 67
  Synthetic
  MISC_FEATURE (1)..(1): optionally absent

SEQ ID NO: 114 - protein, 85 aa, Artificial sequence
  Synthetic
  MISC_FEATURE (1)..(1): optionally absent
GTEETKNSKRVLDIIEELMRQVEENSRELEKRIKELLRQTKEGKTKKELERDVRRTIEEQKKELRRLKEQVRKTKEEQREEQYRS

SEQ ID NO: 115 - protein, 85 aa, Artificial sequence
  Synthetic
  MISC_FEATURE (1)..(1): optionally absent
GTRTEKLMKEVEEIQRRQIELLKKLMKEVEDSSKRNQEATERGTTKKKWKEEQEKILEDLKREVRRIIEESRKWLEDLKKKVYES

SEQ ID NO: 116 - protein, 85 aa, Artificial sequence
  Synthetic
  MISC_FEATURE (1)..(1): optionally absent
GTEKYRLREEVRRTIEEQKENLERLKQEVKETERKTEEWRERNTTTEDAQREQIKIIRRLMKEVERNSRRLEKELRRLVEETRES

SEQ ID NO: 117 - protein, 85 aa, Artificial sequence
  Synthetic
  MISC_FEATURE (1)..(1): optionally absent
GTEKYRLIRESERALRELKRKVRELEEDQRERLDEQRKKVEEGQTTDELLRQNEENSRRMLKETKKLLREIERIQREQQRQNQEN

SEQ ID NO: 118 - protein, 85 aa, Artificial sequence
  Synthetic
  MISC_FEATURE (1)..(1): optionally absent
GTEKEKEIEKNSREVIKQVEDILREIKENSKRNIEIIKELQKDPSDEKMRETIEQQRENLERLERKARELIRRQERNLRETQYKD

SEQ ID NO: 119 - protein, 85 aa, Artificial sequence
  Synthetic
  MISC_FEATURE (1)..(1): optionally absent
GTEKYRIIEEQRRNLEDLEREIREIIKKLKEALERLRELVERNSTNDRLLDEVRKIIEEAIEDMKRLLEKVERSIRQNIEELRRS

SEQ ID NO: 120 - protein, 85 aa, Artificial sequence
  Synthetic
  MISC_FEATURE (1)..(1): optionally absent
GTNKEYLRRKVKELKDQQKRNLEELEREVRRLIKEIEEWRERNTTTDRALKEIIRQIQRLLEEARRNSEEVLRQIEEIMEETRES

SEQ ID NO: 121 - protein, 85 aa, Artificial sequence
  Synthetic
  MISC_FEATURE (1)..(1): optionally absent
GTEEERALERIIRAIRELMREVERNSKEVLQWIKEMLRLTKENSSTKELEERWREIEERQRRNLEKLKEEVRRLEDEIRQETYRS

SEQ ID NO: 122 - protein, 85 aa, Artificial sequence
  Synthetic
  MISC_FEATURE (1)..(1): optionally absent
GTETKKLVEEVERALRELLKTSEDLVRKVEKALRELLELIRRGGTKDKIEEKIRRVLEEIKRELERQKRKIEDVLRQIKEELYRS

SEQ ID NO: 123 - protein, 111 aa, Artificial sequence
  Synthetic
  MISC_FEATURE (110)..(111): optionally absent
SDYLRLATEHNKLAVEANRLAIELAKSAVELAETDPSKTALEHAELAARLLEMMVQFTKAAQELTREAIRKEGRNEESEKVLRKSKEAYKESEKALEDARRLLDELRKKGS

SEQ ID NO: 124 - protein, 111 aa, Artificial sequence
  Synthetic
  MISC_FEATURE (110)..(111): optionally absent
SEELRKAAENNELAVRLAEAALRMARSALHLFEENPSDEMLKFLELAMEVAKMAAELLKASLKMLKKAAEERGSDESVKYLADKSRDIMRQITEELKKLEEEAKRAQKRGS

SEQ ID NO: 125 - protein, 112 aa, Artificial sequence
  Synthetic
  MISC_FEATURE (111)..(112): optionally absent
SEKARIAVENLEAALRLNRAAAEMQKSAIKIMDDNRSDEKALRYLRLTTKVLRMSVELLRASLELAEKALREEGSDDSAEKVRKEAEEILKESTEILKEADKETKRADEEGS

SEQ ID NO: 126 - protein, 112 aa, Artificial sequence
  Synthetic
  MISC_FEATURE (111)..(112): optionally absent
SRRLELAARINKAAAENARSAIEIQELAARLADELSSSKKVIDFARATTEVLRMSVKLLKLSLEMLEEAARQDGRSEEVRYLAEESKKILEEARKALEDADRLTKRIEEEGS

SEQ ID NO: 127 - protein, 112 aa, Artificial sequence
  Synthetic
  MISC_FEATURE (111)..(112): optionally absent
TDVLRIAAENLKAAVELAKAALEMAKSAIEIAKTLTEDDEALKFARAAAEVLRMAAKLLKLSIELARKAAEEEGSDDEVRYILDEARKQADELREALKKVDEIMKELDKRGS

SEQ ID NO: 128 - protein, 70 aa, Artificial sequence
  Synthetic
TRRKQEMKRLKYEMEKIREETEEVKKEIEESKKRPQSESAKNLILIMQLLINQIRLLALQIRMLALQLQE

SEQ ID NO: 129 - protein, 70 aa, Artificial sequence
  Synthetic
TEDQERLRKQMEYERKHTEKVEKEIRKVEQKMKSHEDTSLRLLVLIARLLINQIRLLILQIRSLSNLERN

SEQ ID NO: 130 - protein, 71 aa, Artificial sequence
  Synthetic
TESTLLILIMRLLVQQSELLQLQIQMLQLLLKANNGTNKTEIERRSKEMEEELKRMKESNREMTKRIKEME

SEQ ID NO: 131 - protein, 71 aa, Artificial sequence
  Synthetic
TESDLLRQISKLLIIQIRLLLLQIQMLILLLKMNTGTNTTQITKEAKRIEKEAQEARKELEKMQESNKKQT

SEQ ID NO: 132 - protein, 72 aa, Artificial sequence
  Synthetic
TEDEIRKLRKLLEEAEKKLYKLEDKTRRSEEISKTDDDPKAQSLQLIAESLMLIAESLLIIAISLLLSSRNG

SEQ ID NO: 133 - protein, 72 aa, Artificial sequence
  Synthetic
TEDEELQRVEEEIRELERKAKELHYKSEEIRKKVNGRSPQAEALLMIAQALLNISESLLAIAKALLMIARST

SEQ ID NO: 134 - protein, 70 aa, Artificial sequence
  Synthetic
TDEREIIKRVKRLLEEVEYLIERLRDQIEKAEKGLLDSRKAQQNAEALVNLIKAMVLVLKALLLAKELER

SEQ ID NO: 135 - protein, 70 aa, Artificial sequence
  Synthetic
TEEQYIIEEVKKLLEEVKKLIEELKKQIEKAEKGEEDSRKAQQNAEALVNLIKAMVLVLKALLLAKELER

SEQ ID NO: 136 - DNA, 50 bp, Artificial sequence
  Synthetic
cgccatgctacttatctacggagtcgtgacctagggaacagagaaacagg

SEQ ID NO: 137 - DNA, 24 bp, Artificial sequence
  Synthetic
ttcgaggaagtcaaaacagcgtgg

SEQ ID NO: 138 - DNA, 44 bp, Artificial sequence
  Synthetic
cgctgttttgacttcctcgaaccatgtcttctgtcaagttatgg

SEQ ID NO: 139 - DNA, 56 bp, Artificial sequence
  Synthetic
agaaccactcccttctgatccttcggaacccgtacgcttgtggaagggctgccagc

SEQ ID NO: 140 - DNA, 56 bp, Artificial sequence
  Synthetic
agaaccactcccttctgatccttcggaacccgtacgtcattggattcggtggggtc

SEQ ID NO: 141 - DNA, 39 bp, Artificial sequence
  Synthetic
gatcagaagggagtggttctcatatgggtacgaaatacg

SEQ ID NO: 142 - DNA, 45 bp, Artificial sequence
  Synthetic
cagaggttgattatgcggccttagtcacttttggcgttaattttc

SEQ ID NO: 143 - DNA, 21 bp, Artificial sequence
  Synthetic
ggccgcataatcaacctctgg

SEQ ID NO: 144 - DNA, 43 bp, Artificial sequence
  Synthetic
ccgccatgctacttatctacaaaaaacctcccacatctccccc

SEQ ID NO: 145 - DNA, 21 bp, Artificial sequence
  Synthetic
ggaacccctagtgatggagtt

SEQ ID NO: 146 - DNA, 16 bp, Artificial sequence
  Synthetic
cggcctcagtgagcga

SEQ ID NO: 147 - protein, 77 aa, Artificial sequence
  Synthetic
HMGTKYELRRALEELEKALRELKKSLDELERSLEELEKNPSEDALVENNRLNVENNKIIVEVLRIIAEVLKINAKSD

SEQ ID NO: 148 - protein, 75 aa, Artificial sequence
  Synthetic
GTKYEIERMLKELKDLLEELKRMLEELKRSLKELKKNPSEDALVENNRLIVKVLEVIVEVLRAIIELAELIIRSD

SEQ ID NO: 149 - protein, 72 aa, Artificial sequence; identical to SEQ ID NO: 132
  Synthetic

SEQ ID NO: 150 - protein, 11 aa, Artificial sequence
  Synthetic
GSEGSEGSEGS

SEQ ID NO: 151 - protein, 6 aa, Artificial sequence
  Synthetic
GSGSGS

SEQ ID NO: 152 - protein, 292 aa, Homo sapiens
VEHSLDNKDGPVCLQVKPCTPEFYQTHFQLAYRLQSRPRGLALVLSNVHFTGEKELEFRSGGDVDHSTLVTLFKLLGYDVHVLCDQTAQEMQEKLQNFAQLPAHRVTDSCIVALLSHGVEGAIYGVDGKLLQLQEVFQLFDNANCPSLQNKPKMFFIQACRGDETDRGVDQQDGKNHAGSPGCEESDAGKEKLPKMRLPTRSDMICGYACLKGTAAMRNTKRGSWYIEALAQVFSERACDMHVADMLVKVNALIKDREGYAPGTEFHRCKEMSEYCSTLCRHLYLFPGHPPT

SEQ ID NO: 153 - protein, 160 aa, Homo sapiens
MAAPSAGSWSTFQHKELMAADRGRRILGVCGMHPHHQETLKKNRVVLAKQLLLSELLEHLLEKDIITLEMRELIQAKVGSFSQNVELLNLLPKRGPQAFDAFCEALRETKQGHLEDMLLTTLSGLQHVLPPLSCDYDLSLPFPVCESCPLYKKLRLSTDT

SEQ ID NO: 154 - protein, 452 aa, Homo sapiens
MAAPSAGSWSTFQHKELMAADRGRRILGVCGMHPHHQETLKKNRVVLAKQLLLSELLEHLLEKDIITLEMRELIQAKVGSFSQNVELLNLLPKRGPQAFDAFCEALRETKQGHLEDMLLTTLSGLQHVLPPLSCDYDLSLPFPVCESCPLYKKLRLSTDTVEHSLDNKDGPVCLQVKPCTPEFYQTHFQLAYRLQSRPRGLALVLSNVHFTGEKELEFRSGGDVDHSTLVTLFKLLGYDVHVLCDQTAQEMQEKLQNFAQLPAHRVTDSCIVALLSHGVEGAIYGVDGKLLQLQEVFQLFDNANCPSLQNKPKMFFIQACRGDETDRGVDQQDGKNHAGSPGCEESDAGKEKLPKMRLPTRSDMICGYACLKGTAAMRNTKRGSWYIEALAQVFSERACDMHVADMLVKVNALIKDREGYAPGTEFHRCKEMSEYCSTLCRHLYLFPGHPPT

SEQ ID NO: 155 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 156 - protein, 314 aa, Homo sapiens
QGEEELVSQTDVKTFLEALPQESWQNKHAGSNGNRATNGAPSLVSRGMQGASANTLNSETSTKRAAVYRMNRNHRGLCVIVNNHSFTSLKDRQGTHKDAEILSHVFQWLGFTVHIHNNVTKVEMEMVLQKQKCNPAHADGDCFVFCILTHGRFGAVYSSDEALIPIREIMSHFTALQCPRLAEKPKLFFIQACQGEEIQPSVSIEADALNPEQAPTSLQDSIPAEADFLLGLATVPGYVSFRHVEEGSWYIQSLCNHLKKLVPRHEDILSILTAVNDDVSRRVDKQGTKKQMPQPAFTLRKKLVFPVPLDALSL

SEQ ID NO: 157 - protein, 208 aa, Homo sapiens
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEKYKREKAIQIVTPPVDKEAESY

SEQ ID NO: 158 - protein, 522 aa, Homo sapiens
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEKYKREKAIQIVTPPVDKEAESYQGEEELVSQTDVKTFLEALPQESWQNKHAGSNGNRATNGAPSLVSRGMQGASANTLNSETSTKRAAVYRMNRNHRGLCVIVNNHSFTSLKDRQGTHKDAEILSHVFQWLGFTVHIHNNVTKVEMEMVLQKQKCNPAHADGDCFVFCILTHGRFGAVYSSDEALIPIREIMSHFTALQCPRLAEKPKLFFIQACQGEEIQPSVSIEADALNPEQAPTSLQDSIPAEADFLLGLATVPGYVSFRHVEEGSWYIQSLCNHLKKLVPRHEDILSILTAVNDDVSRRVDKQGTKKQMPQPAFTLRKKLVFPVPLDALSL

SEQ ID NO: 159 - protein, 244 aa, Mus musculus
RTRKALIICNTEFKHLSLRYGANFDIIGMKGLLEDLGYDVVVKEELTAEGMESEMKDFAALSEHQTSDSTFLVLMSHGTLHGICGTMHSEKTPDVLQYDTIYQIFNNCHCPGLRDKPKVIIVQACRGGNSGEMWIRESSKPQLCRGVDLPRNMEADAVKLSHVEKDFIAFYSTTPHHLSYRDKTGGSYFITRLISCFRKHACSCHLFDIFLKVQQSFEKASIHSQMPTIDRATLTRYFYLFPGN

SEQ ID NO: 160 - protein, 128 aa, Mus musculus
MAENKHPDKPLKVLEQLGKEVLTEYLEKLVQSNVLKLKEEDKQKFNNAERSDKRWVFVDAMKKKHSKVGEMLLQTFFSVDPGSHHGEANLEMEEPEESLNTLKLCSPEEFTRLCREKTQEIYPIKEAN

SEQ ID NO: 161 - protein, 373 aa, Mus musculus
MAENKHPDKPLKVLEQLGKEVLTEYLEKLVQSNVLKLKEEDKQKFNNAERSDKRWVFVDAMKKKHSKVGEMLLQTFFSVDPGSHHGEANLEMEEPEESLNTLKLCSPEEFTRLCREKTQEIYPIKEANGRTRKALIICNTEFKHLSLRYGANFDIIGMKGLLEDLGYDVVVKEELTAEGMESEMKDFAALSEHQTSDSTFLVLMSHGTLHGICGTMHSEKTPDVLQYDTIYQIFNNCHCPGLRDKPKVIIVQACRGGNSGEMWIRESSKPQLCRGVDLPRNMEADAVKLSHVEKDFIAFYSTTPHHLSYRDKTGGSYFITRLISCFRKHACSCHLFDIFLKVQQSFEKASIHSQMPTIDRATLTRYFYLFPGN

(19) United States
(12) Patent Application Publication
(10) Pub. No.: US 2021/0040469 A1
(43) Pub. Date: Feb. 11, 2021

(54) ENGINEERED CELL DEATH-INDUCING ENZYMES AND METHODS OF USE

(71) Applicant: University of Washington, Seattle, WA (US)

(72) Inventors: Andrew Oberst, Seattle, WA (US); Nicholas Hubbard, Seattle, WA (US); Annelise Snyder, Seattle, WA (US)

(73) Assignee: University of Washington, Seattle, WA (US)

(21) Appl. No.: 16/964,936

(22) PCT Filed: Jan. 24, 2019
(86) PCT No.: PCT/US2019/014995

§ 371 Date: Jul. 24, 2020

Related U.S. Application Data
(60) Provisional application No. 62/622,049, filed on Jan. 25, 2018.

(51) Int. Cl. C12N 9/64; C12N 9/12; C12N 15/62; A61K 35/33; A61K 45/06; A61P 35/00; A61P 37/04

(52) U.S. Cl. CPC C12N 9/6472; C12Y 304/22062; C12N 9/12; C12Y 207/10002; A61P 37/04; A61K 35/33; A61K 45/06; A61P 35/00; C12N 15/62

(57) ABSTRACT

The disclosure provides compositions and methods for inducing programmed cell death, such as necroptosis. Compositions may comprise fusion proteins comprising a death inducing domain and a multimerization domain; nucleic acids encoding fusion proteins; and cells comprising fusion proteins. The compositions may be used in methods such as cancer therapy, including in combination with additional immunotherapeutics.

### CROSS-REFERENCE(S) TO RELATED APPLICATION

This application claims the benefit of Provisional Application No. 62/622,049, filed Jan. 25, 2018, the disclosure of which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 68084_Seq_Listing_Final_20190124.txt. The text file is 250 KB; was created on Jan. 24, 2019; and is being submitted via EFS-Web with the filing of the specification.

### BACKGROUND

Cancers are characterized by a cell's loss of the ability to control the cell cycle, resulting in abnormal and uncontrolled cell growth. The transformed cells often lose the ability to respond to typical signals that induce programmed cell death (PCD) in healthy cells.

Programmed cell death (PCD) is a process by which cells can activate specific enzymes that lead to their demise. There are multiple forms of programmed cell death, including apoptosis, pyroptosis, and necroptosis. Of these, apoptosis is the best-understood, and is considered nonimmunogenic, while pyroptosis and necroptosis, more recently-described, can promote inflammation and immunity. These processes are carried out by specific enzymes, notably members of the caspase family of cysteine proteases (apoptosis and pyroptosis), by the receptor-interacting protein kinases (RIPKs), the pseudokinase MLKL (necroptosis) and the pore-forming molecules of the Gasdermin family. Importantly, all these enzymes are present in healthy cells as inactive zymogens, and are activated by induced-proximity; that is, inactive precursors come together to form active enzymes, which activate cell death pathways.

Induction of PCD represents a strategy to check growth of cell populations that can be applicable to cancer therapeutic strategies. While signals leading to different types of PCD have been described, there remains a need for compositions and methods for facile and reliable induction of PCD across a variety of cells types, including transformed (e.g., tumor) cells. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a fusion protein comprising a death inducing domain and a multimerization domain. In one embodiment, the death inducing domain and the multimerization domain do not naturally occur together in the same protein. In one embodiment, the death inducing domain induces programmed cell death when the fusion protein forms a complex with a protein comprising one or more further death inducing domains. In one embodiment, the programmed cell death is selected from apoptosis, pyroptosis, and necroptosis.

In one embodiment, the death inducing domain comprises an effector domain selected from: a caspase effector domain, a kinase effector domain, a MLKL effector domain, and a gasdermin effector domain. In a one embodiment, the death inducing domain comprises a caspase effector domain selected from: a caspase 1 effector domain, a caspase 2 effector domain, a caspase 3 effector domain, a caspase 4 effector domain, a caspase 5 effector domain, a caspase 8 effector domain, a caspase 9 effector domain, a caspase 10 effector domain, and a caspase 11 effector domain. In a further embodiment, the fusion protein further comprises a caspase prodomain.

In one embodiment, the death inducing domain comprises a kinase effector domain selected from: a RIPK1 kinase effector domain, a RIPK2 kinase effector domain, and a RIPK3 kinase effector domain. In one embodiment, the fusion protein further comprises a RIP homotypic interaction motif (RHIM) domain.

In one embodiment, the death inducing domain comprises an MLKL effector domain. In one embodiment, the fusion protein further comprises a pseudokinase domain.

In one embodiment, the death inducing domain comprises a gasdermin effector domain. In one embodiment, the fusion protein further comprises an auto-inhibitory domain.

In some embodiments of the fusion protein, the multimerization domain promotes formation of a complex comprising the fusion protein and one or more further proteins comprising the same multimerization domain or a different multimerization domain. In some embodiments, the multimerization domain comprises: a dimerization domain, a trimerization domain, a tetramerization domain, a pentamerization domain, a heptamerization domain, an octamerization domain, or any combination of the above. In some embodiments, the multimerization domain comprises a domain selected from: 2LHC2-23, 5L6HC3-1, 2L6HC3-9, 2L6HC3-13, 6H8, and 7H3.

In some embodiments of the fusion protein, the death inducing domain and the multimerization domain are joined by a linker domain. In some embodiments, the linker domain is a flexible amino acid linker domain. In some embodiments, the linker domain comprises one or more alanine residues, serine residues, glycine residues, or a combination thereof.

In some embodiments, the fusion protein forms a complex with one or more copies of the same fusion protein.

In another aspect, the disclosure provides a nucleic acid comprising a sequence encoding the fusion protein disclosed herein. In some embodiments, the nucleic acid further comprises a promoter sequence operatively linked to the sequence encoding the fusion protein.

In another aspect, the disclosure provides a vector comprising the nucleic acid disclosed herein. In one embodiment, the vector is a viral vector, a circularized nucleic acid, or a nanoparticle. In one embodiment, the vector is a viral vector selected from an adeno associated virus (AAV) vector, an adenovirus vector, a retrovirus vector, and a lentivirus vector. In one embodiment, the vector is an AAV2.5 viral vector.

In another aspect, the disclosure provides a cell comprising the nucleic acid disclosed herein, or the vector disclosed herein. The cell can be a tumor cell, a tumor-associated stromal cell, or other cell.

In another aspect, the disclosure provides a method of inducing programmed cell death in a cell. The method comprises providing the cell with multiple copies of the fusion protein disclosed herein. In one embodiment, the step of providing the cell with multiple copies of the fusion protein comprises contacting the cell with the nucleic acid disclosed herein or the vector disclosed herein under conditions that permit expression of the fusion protein in the cell. In one embodiment, the programmed cell death induced in the cell is selected from apoptosis, pyroptosis, and necroptosis.

In one embodiment, the death inducing domain is a caspase effector domain selected from: a caspase 2 effector domain, a caspase 3 effector domain, a caspase 8 effector domain, a caspase 9 effector domain, and a caspase 10 effector domain, and wherein the programmed cell death induced in the cell is apoptosis. In one embodiment, the death inducing domain is a caspase effector domain selected from: a caspase 1 effector domain, a caspase 4 effector domain, a caspase 5 effector domain, a caspase 9 effector domain, and a caspase 11 effector domain, and wherein the programmed cell death induced in the cell is pyroptosis. In one embodiment, the cell death domain is a gasdermin effector domain, and wherein the programmed cell death induced in the cell is pyroptosis. In one embodiment, the death inducing domain is selected from: a RIPK1 kinase effector domain, a RIPK2 kinase effector domain, a RIPK3 kinase effector domain, and a MLKL effector domain, and wherein the programmed cell death induced in the cell is necroptosis.

In some embodiments of the method, the cell is a tumor cell, a tumor-associated stromal cell, or other cell. In some embodiments of the method, the programmed cell death is induced in the cell in vitro. In some embodiments of the method, the programmed cell death is induced in the cell in vivo in a subject with a tumor or cancer.

In another aspect, the disclosure provides a method of inhibiting tumor cell growth in a subject. The method comprises administering to the subject an effective amount of the nucleic acid disclosed herein or the vector disclosed herein. In one embodiment, the nucleic acid or vector is administered under conditions that permit expression of the fusion protein in a tumor cell or tumor-associated stromal cell. In one embodiment, the nucleic acid or vector is administered intratumorally. In one embodiment, the method further comprises administering to the subject an effective amount of an immunotherapeutic agent. In one embodiment, the immunotherapeutic agent is a checkpoint inhibitor. In one embodiment, the checkpoint inhibitor is a PD-1 pathway inhibitor. In one embodiment, the PD-1 pathway inhibitor is an anti-PD-1 antibody, an anti-PD-L1 antibody (or a functional fragment or derivative thereof).

In another aspect, the method provides a method of inhibiting growth of tumor cells in a subject. The method comprises modifying the tumor microenvironment of the tumor cells to contain necroptotic cells, wherein the necroptotic cells comprise multimers of an engineered protein comprising a death inducing domain that induces necroptosis.

In one embodiment, the presence of necroptotic cells within the tumor microenvironment (i) promotes an anti-tumor immune response in the subject, (ii) increases the number of CD8+ T-cells, (iii) increases antigen uptake or activation of tumor-associated antigen presenting cells, (iv) upregulates NF-κB-dependent gene expression, (v) upregulates expression of inflammatory chemokines or cytokines, or (vi) a combination of any of (i)-(v). In one embodiment, the engineered protein is a fusion protein as disclosed herein. In one embodiment, the death inducing domain comprises a RIPK1 kinase effector domain, a RIPK2 kinase effector domain, a RIPK3 kinase effector domain, or a MLKL effector domain. In one embodiment, the fusion protein further comprises a RHIM domain. In one embodiment, the step of modifying the tumor microenvironment comprises: generating necroptotic cells in vitro, and administering the necroptotic cells to the tumor microenvironment prior to the death of at least some of the necroptotic cells. In one embodiment, the necroptotic cells are generated by expressing the engineered protein in cells under conditions that permit multimerization of the engineered protein. In one embodiment, the engineered protein is expressed in the cells by contacting the cells with the nucleic acid disclosed herein or the vector disclosed herein. In one embodiment, the cells are autologous with respect to the tumor cells in the tumor microenvironment. In one embodiment, the cells are tumor cells or tumor-associated stromal cells that are obtained from the tumor microenvironment prior to generating necroptotic cells therefrom. In one embodiment, the cells are administered by intratumoral injection. In one embodiment, the step of modifying the tumor microenvironment comprises exposing the tumor microenvironment to the nucleic acid disclosed herein or the vector disclosed herein in vivo.

In one embodiment, the tumor microenvironment is exposed to the vector of any one disclosed herein, and wherein the vector is an AAV. In one embodiment, the tumor microenvironment is exposed to the nucleic acid or vector by intratumoral injection.

In one embodiment, the method further comprises administering to the subject an effective amount of an immunotherapeutic agent. In one embodiment, the immunotherapeutic agent is a PD-1 pathway inhibitor. In one embodiment, the PD-1 pathway inhibitor is an anti-PD-1 antibody or an anti-PD-L1 antibody (or functional fragment or derivative thereof).

In another aspect, the disclosure provides a fusion protein comprising a death inducing domain and a multimerization domain, wherein the death inducing domain comprises a human RIPK3 kinase effector domain, and the multimerization domain is a trimerization domain. In one embodiment, the fusion protein comprises amino acid sequence of SEQ ID NO:53. In one embodiment, the fusion protein further comprises a RHIM domain. In one embodiment, the fusion protein further comprising the amino acid sequence of SEQ ID NO:52.

In another embodiment, the disclosure provides a method of treating a subject with a tumor comprising administering to the subject a vector comprising a nucleic acid that encodes the fusion protein as disclosed herein. In one embodiment, the fusion protein comprises a death inducing domain and a multimerization domain, wherein the death inducing domain comprises a human RIPK3 kinase effector domain, and the multimerization domain is a trimerization domain. In one embodiment, the fusion protein comprises amino acid sequence of SEQ ID NO:53. In one embodiment, the fusion protein further comprises a RHIM domain. In one embodiment, the fusion protein further comprising the amino acid sequence of SEQ ID NO:52. In one embodiment, the vector is an adenovirus vector, such as AAV2.5.

In another aspect, the disclosure provides a method of treating a subject with a tumor, comprising administering to the subject a cell comprising the fusion protein disclosed herein. In one embodiment, the fusion protein comprises a death inducing domain and a multimerization domain, wherein the death inducing domain comprises a human RIPK3 kinase effector domain, and the multimerization domain is a trimerization domain. In one embodiment, the cell is a tumor cell, a tumor-associated stromal cell, or other cell. In one embodiment, the other cell is a fibroblast. In one embodiment, the cell is autologous with respect to the subject. In one embodiment, the tumor is a solid tumor selected from pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, and soft tissue sarcoma.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic comparing the endogenous pathway for proximity induced induction of cell programmed death with exemplary embodiments of the engineered, constitutively active cell death effectors.

FIGS. 2A-2C schematically and graphically illustrate the engineered multimerizing Caspase death inducing domains and their induction of death in cells.

FIGS. 3A and 3B illustrate results of a similar approach to that described in FIGS. 2A-2C, but incorporating the necroptosis inducing enzyme RIPK3.

FIGS. 4A-4F illustrate that intratumoral administration of necroptotic cells confers control of both primary and distal syngeneic flank tumors. FIGS. 4A-4E illustrate tumor growth and survival of B6/J mice bearing B16.F10-OVA (FIGS. 4A and 4C), LL/2-OVA (FIGS. 4B and 4D), or E.G7-OVA (FIG. 4E) flank tumors following administration of apoptotic or necroptotic autologous (FIGS. 4A and 4B) or unmatched (FIGS. 4C-4E) NIH-3T3 fibroblast cells. N=10-16 mice per group. FIG. 4F illustrates tumor growth of ipsilateral (“I”, treated) and contralateral (“C”, untreated) B16.F10-OVA tumors following administration of either apoptotic or necroptotic NIH-3T3 cells. N=9-11 mice per group. ****p<0.0001. Black arrows indicate intratumoral dying cell injections. Error bars represent SEM. Data are pooled from 3-5 independent experiments.

FIGS. 5A-5H graphically illustrate that tumor control by necroptotic fibroblasts requires Batf3⁺ cDC1 and CD8⁺ leukocytes, and occurs independently of DAMP sensing or systemic inflammation. FIGS. 5A and 5B illustrate that tumor growth and survival following administration of lytic necrotic fibroblasts in single (FIG. 5A), or bilateral (FIG. 5B) B16.F10-OVA flank tumors. Tumor growth and survival curves for PBS, acCASP8, and acRIPK3 as presented in FIGS. 4A-4F are also graphed for comparison. RIPK3ΔC, MLKL, and freeze/thaw conditions all represent forms of lytic necrotic NIH-3T3 fibroblasts. N=9-16 mice per group. FIG. 5C graphically illustrates day 12 volumes of B16.F10-OVA tumors in various innate immune knockout mice following necroptotic fibroblast injections on days 6, 8, and 10. N=5-15 mice per group. FIG. 5D graphically illustrates assessment of systemic inflammation via Luminex assay for inflammatory serum cytokines and chemokines 48 hours post-intratumoral dying NIH-3T3 injection. N=3-5 mice per group. FIG. 5E illustrates B16.F10-OVA tumor growth curves following intratumoral (IT), intraperitoneal (IP), distal subcutaneous (distal subQ), or intravenous (IV) injection of necroptotic fibroblasts. N=7-9 mice per group. FIG. 5F illustrates tumor growth (left panel) and survival (right panel) of B16.F10-OVA tumor-bearing mice following co-administration of necroptotic fibroblasts with the leukocyte trafficking inhibitor FTY-720. N=8-12 mice per group. FIG. 5G illustrates Day 12 B16.F10-OVA tumor volumes (left panel) and survival (right panel) of mice with varying Batf3 genotypes following necroptotic fibroblast injections on days 6, 8, and 10. N=4-12 mice per group. FIG. 5H illustrates B16.F10-OVA tumor growth upon co-administration of necroptotic fibroblasts with depleting antibodies against either CD4⁺ or CD8⁺ leukocytes. N=6-11 mice per group. *p<0.05, **p<0.01, ***p<0.001, ****p<0.0001. OOR> represents out of range detection value. Black arrows indicate intratumoral dying cell injections. Error bars represent SEM. Data are pooled from 2-5 independent experiments.

FIGS. 6A-6F graphically illustrate that necroptotic fibroblast administration is associated with expansion of beneficial cytotoxic CD8⁺ T cells in the tumor and tumor-draining lymph node, and synergizes with co-administration of α-PD-1 to promote durable tumor clearance. FIG. 6A illustrates absolute numbers of intratumoral CD8⁺ T cells with various phenotyping markers for proliferation (Ki67), effector function (GranzymeB, GzmB), and activation (CD44), normalized per gram of tumor tissue. FIG. 6B illustrates quantification of the ratio of intratumoral activated (CD44^{hi}, left panel) or tumor antigen-specific (SIINFEKL-H2K^{b+}, right panel) CD8⁺ T cells to immunosuppressive Foxp3⁺ CD25⁺ T_{REG}, normalized per gram of tumor tissue. FIG. 6C illustrates quantified percentages of overall activated CD8⁺ T cells in the tumor-draining (inguinal) lymph node. FIG. 6D illustrates quantified percentages of OVA-specific and activated CD8⁺ T cells in the tumor-draining (inguinal) lymph node. FIG. 6D illustrates survival curves of B16.F10-OVA tumor-bearing mice following co-administration of intratumoral necroptotic NIH-3T3 fibroblasts with the immune checkpoint blockade reagent α-PD-1 or IgG2a isotype. N=8-14 mice per group. FIG. 6F, left panel, illustrates schematic of tumor re-challenge experiments in mice from FIG. 6E that successfully clear B16.F10-OVA tumors. FIG. 6F, right panel, illustrates the survival of mice re-challenged with B16.F10-OVA cells on the same flank as initial tumor location. N=10 mice per group. *p<0.05, **p<0.01, ***p<0.001, ****p<0.0001. All flow harvests performed 48 hours post-dying cell injection. Error bars represent SEM. Data are pooled from 3-4 independent experiments.

FIGS. 7A-7G graphically illustrate that necroptotic fibroblast administration correlates with increased infiltration of CD103⁺ DCs into the tumor microenvironment, increased percentages tumor antigen loading by tuAPCs, and increased activation status of tuAPC subsets. FIG. 7A illustrates absolute numbers of tumor-associated myeloid cell subsets following intratumoral dying cell administration, normalized per gram of tumor tissue. N=6-10 mice per group. FIG. 7B illustrates tumor growth curves of B16.F10-OVA tumor-bearing mice following co-administration of intratumoral necroptotic NIH-3T3 fibroblasts with depleting antibodies against NK1.1⁺ NK cells. N=7-10 mice per group. FIG. 7C illustrates experimental schematic of B16.F10-OVA tumor cells expressing zsGreen as a surrogate tumor antigen, allowing for gating on tuAPCs that have phagocytosed tumor antigen. FIG. 7D illustrates percent of zsGreen⁺ tuAPCs following intratumoral dying cell administration. FIG. 7E illustrates geometric mean fluorescence intensity (gMFI) of the costimulatory marker CD80 on zsGreen⁺ subsets of tuAPC populations. N=3-4 mice per group. FIG. 7F illustrates quantification of previously activated OT-I T cell proliferation upon co-culture with zsGreen⁺ tuAPC subsets sorted ex vivo from B16.F10-OVA-zsGreen tumors following dying cell injection. N=3 technical replicates per group, using pooled tuAPCs from 5 mice per treatment group. FIG. 7G illustrates in vitro characterization of bone-marrow derived macrophages (BMDMs) co-cultured with live or necroptotic B16.F10-zsGreen tumor cells and dextran-fluorophore beads, assessed for phagocytosis via dextran uptake (left panel), and expression of costimulatory marker CD80 gMFI in zsGreen⁺ BMDMs (middle panel) or dextran⁺ beads (right panel). N=3 technical replicates per group. *p<0.05, **p<0.01, ***p<0.001, ****p<0.0001. All flow harvests performed 48 hours post-dying cell injection. Error bars represent SEM. Data are representative plots from 2-3 independent experiments (FIGS. 7E-7G), or pooled from 2-3 independent experiments (FIGS. 7A, 7B, and 7D).

FIGS. 8A-8D illustrate that the disclosed engineered self-complementing adeno-associated viruses (AAVs) induce necroptosis in tumor cells in vitro. FIG. 8A illustrates a schematic of AAVs used to transduce tumor cells to express engineered pro-death enzymes fused to a constitutively-oligomerizing (“.co”) recruitment domain under the control of a synthetic MND promoter, leading to subsequent induction of a corresponding PCD modality. For example, AAV transduction of RIPK3.co induces necroptosis. FIGS. 8B and 8C illustrate validation and kinetics of AAV2.5 serotype transduction efficiency in B16.F10 cells in vitro. FIG. 8B shows the percent of GFP⁺ cells transduced with AAV2.5-eGFP control. FIG. 8C shows the percent cell death in cells transduced with various death-inducing constructs. N=3 technical replicates per group. FIG. 8D illustrates a heat map depicting relative expression values of NF-κB-dependent gene targets, inflammatory chemokines, and inflammatory cytokines via Nanostring analysis of B16.F10 tumor cells compared to eGFP-transduced controls 10 hours following AAV2.5 transduction (1×10¹¹ IFU). Error bars represent SEM. Data are representative plots from 2 independent experiments (FIGS. 8B and 8C), or mean of 3 biological replicates from 1 experiment (FIG. 8D).

FIGS. 9A-9F graphically illustrate that adeno-associated viruses (AAVs) target necroptosis in tumor cells in situ, conferring tumor growth restriction that synergizes with immune checkpoint blockade to promote durable tumor clearance. FIG. 9A illustrates tumor growth of ipsilateral (“I”, treated) and contralateral (“C”, untreated) B16.F10-OVA tumors following intratumoral administration of death-inducing AAVs or eGFP control AAV. N=10-12 mice per group. FIG. 9B illustrates survival curves of B16.F10-OVA tumor-bearing mice following co-administration of intratumoral AAVs with IgG2a isotype control antibody. N=14-15 mice per group. FIG. 9C illustrates survival curves of B16.F10-OVA tumor-bearing mice following co-administration of intratumoral AAVs with the immune checkpoint blockade reagent α-PD-1. N=13-16 mice per group. FIG. 9D illustrates B16.F10-OVA tumor growth upon co-administration of necroptosis-inducing RIPK3.co AAV with depleting antibodies against CD8⁺ leukocytes. N=3-8 mice per group. FIG. 9E illustrates B16.F10-OVA tumor growth in Batf3^{-/-} or wild-type control mice following necroptosis-inducing AAV administration. N=10-13 mice per group. FIG. 9F, left panel, is a schematic of tumor re-challenge experiments in mice from FIG. 9C that successfully clear B16.F10-OVA tumors. FIG. 9F, right panel, illustrates survival of mice re-challenged with B16.F10-OVA cells on the same flank as initial tumor location. N=8-10 mice per group. FIG. 9G illustrates Kaplan-Meier plot for overall survival of skin cutaneous melanoma patients in TCGA data set. Data are parsed on upper and lower quartiles (25% ile) of RIPK3 mRNA expression. N=114 patients per group. *p<0.05, **p<0.01, ***p<0.001, ****p<0.0001. Black arrows indicate intratumoral AAV injections. Error bars represent SEM. Data are pooled from 2-4 independent experiments (FIGS. 9A-9F).

FIGS. 10A-10F (related to FIGS. 4A-4F) illustrate that intratumoral necroptotic cell administration extends survival of tumor-bearing mice. FIGS. 10A and 10B illustrate median survival of B6/J mice bearing B16.F10-OVA (FIG. 10A) or LL/2-OVA (FIG. 10B) flank tumors following administration of autologous apoptotic or necroptotic tumor cells. FIGS. 10C-10E illustrate survival of mice B6/J mice bearing B16.F10-OVA (FIG. 10C), LL/2-OVA (FIG. 10D), or E.G7-OVA (FIG. 10E) flank tumors following administration of dying unmatched NIH-3T3 fibroblast cells. FIG. 10F illustrates survival curves and median survival time of B6/J mice bearing bilateral B16.F10-OVA tumors that received intratumoral injection of dying unmatched NIH-3T3 fibroblast cells. ****p<0.0001. Black arrows indicate intratumoral dying cell injections. Error bars represent SEM. Data are pooled from 3-5 independent experiments.

FIGS. 11A-11H (related to FIGS. 5A-5H) illustrate that tumor control requirements are recapitulated in multiple syngeneic flank tumor models. FIG. 11A illustrates growth curves of LL/2-OVA (left panel) or E.G7-OVA (right panel) flank tumors receiving intratumoral lytic necrotic (RIPK3ΔC) fibroblasts. FIG. 11B illustrates survival of B16.F10-OVA tumor-bearing mice following treatment with intratumoral lytic necrotic (RIPK3ΔC) fibroblasts. FIG. 11C illustrates day 12 volumes of LL/2-OVA tumors in various innate immune knockout mice following necroptotic fibroblast injections on days 6, 8, and 10 (left panel). FIG. 11D illustrates assessment of systemic inflammation via Luminex assay for inflammatory serum cytokines and chemokines typically associated with necroptotic NIH-3T3 cells, 48 hours post-intratumoral dying NIH-3T3 injection. FIG. 11E illustrates survival curves (left panel) and median survival (right panel) of B16.F10-OVA tumor-bearing mice following intratumoral (IT), intraperitoneal (IP), distal subcutaneous (distal subQ), or intravenous (IV) injection of necroptotic fibroblasts. FIG. 11F illustrates absolute numbers of lymphocyte subsets isolated from B16.F10-OVA tumors 48 hours post-dying NIH-3T3 injection. FIG. 11G illustrates survival of LL/2-OVA tumor-bearing mice with varying Batf3 genotypes following necroptotic fibroblast injections on days 6, 8, and 10. FIG. 11H illustrates survival of LL/2-OVA tumor-bearing mice upon co-administration of necroptotic fibroblasts with depleting antibodies against either CD4⁺ or CD8⁺ leukocytes. *p<0.05, **p<0.01, ***p<0.001, ****p<0.0001. OOR> represents out of range detection value; bar represents upper limit of detection. Black arrows indicate intratumoral dying cell injections. Error bars represent SEM. Data are pooled from 2-5 independent experiments.

FIGS. 12A-12D (related to FIGS. 6A-6F) graphically illustrate further quantification of T cell subsets in tumor and tumor-draining lymph node following dying cell administration. FIG. 12A illustrates absolute numbers of various CD8⁺ T cells isolated from tumor tissue. FIG. 12B illustrates absolute numbers of overall activated (CD44^{hi}CD62L^{lo}, left panel) and activated, OVA-specific (CD44^{hi} SIINFEKL-H2K^{b+}, right panel) CD8⁺ T cells in the tumor-draining (inguinal) lymph node. FIG. 12C illustrates percent of mice that successfully cleared B16.F10-OVA tumors following co-administration of necroptotic fibroblasts with α-PD-1. N=8-14 mice per group. FIG. 12D illustrates percent of mice in different treatment groups that grew B16.F10-OVA tumors following tumor re-challenge. N=10 mice per group. **p<0.01, ***p<0.001, ****p<0.0001. All flow harvests performed 48 hours post-dying cell injection. Error bars represent SEM. Data are pooled from 3-4 independent experiments.

FIGS. 13A-13F (related to FIGS. 7A-7G) illustrate that necroptotic fibroblast administration correlates with increased infiltration of CD103⁺ DCs into the tumor microenvironment, increased percentages tumor antigen loading in tuAPCs, and increased activation status of tuAPC subsets. FIG. 13A illustrates absolute numbers of tumor-associated myeloid cell subsets following intratumoral dying cell administration, normalized per gram of tumor tissue. N=6-10 mice per group. FIG. 13B illustrates survival of B16.F10-OVA tumor-bearing mice following co-administration of intratumoral necroptotic NIH-3T3 fibroblasts with depleting antibodies against NK1.1⁺ NK cells. N=7-10 mice per group. FIG. 13C illustrates absolute numbers of zsGreen⁺ tuAPCs following intratumoral dying cell administration. FIG. 13D illustrates gMFI of the costimulatory marker CD80 on bulk (zsGreen⁺ and zsGreen⁻) subsets of tuAPC populations. FIG. 13E illustrates representative histograms showing sample gating on proliferated (CellTrace Violet^{lo}), previously-activated OT-I T cells. FIG. 13F illustrates in vitro assessment of immunosuppressive CD206 (left panel) and VCAM-1 (right panel) expression on bone-marrow derived macrophages (BMDMs) co-cultured with live or necroptotic B16.F10-zsGreen tumor cells and dextran-fluorophore beads. N=3 technical replicates per group. *p<0.05, **p<0.01, ****p<0.0001. All flow harvests performed 48 hours post-dying cell injection. Error bars represent SEM. Data are representative plots from 2-3 independent experiments (FIGS. 13D and 13F), or pooled from 2-3 independent experiments (FIGS. 13A, 13C, and 13H).

FIGS. 14A-14E (related to FIGS. 8A-8D) illustrate that the disclosed engineered self-complementing adeno-associated viruses (AAVs) induce necroptosis in tumor cells in vitro. FIG. 14A illustrates a summary of AAVs tested. Caspase-9.co denotes fusion to a 2L4HC2-23 dimerization domain that is sufficient for apoptosis induction, while RIPK3.co and RIPKΔC.co denote target protein fusion to a 2L6HC3-9 trimerization domain that is sufficient for necroptosis or lytic necrosis induction, respectively. FIG. 14B illustrates percentage of GFP transduction in non-leukocytic (CD45⁻) and various immune cell populations isolated from B16.F10-OVA tumors 48 hours following intratumoral injection of 1×10¹¹ IFU of different AAV serotypes encoding eGFP. N=5-10 mice per group. FIG. 14C illustrates confirmation of PCD modality dependence upon expected signaling components in B16.F10 cells. zVAD=pan-caspase inhibitor zVAD-fmk, which inhibits caspase-dependent apoptosis (top panel). GSK843=murine RIPK3 inhibitor, which inhibits RIPK3-mediated necroptosis and RIPK3ΔC-mediated lytic necrosis. Cells transduced with RIPK3.co undergo caspase-8 dependent apoptosis in the presence of GSK843 alone (middle panel), which is eliminated upon co-administration of GSK843+zVAD (bottom panel). N=3 technical replicates per group. FIG. 14D illustrates heat map depicting relative expression values of genes via Nanostring analysis of B16.F10 tumor cells compared to eGFP-transduced controls 10 hours following AAV2.5 transduction (1×10¹¹ IFU). FIG. 14E illustrates qRT-PCR analysis of target gene mRNA expression in B16.F10 tumor cells 10 hours following AAV2.5 transduction (1×10¹¹ IFU). Error bars represent SEM. Data are representative plots from 2 independent experiments (FIGS. 14B and 14C), or mean of 3 biological replicates from 1 experiment (FIG. 14D), or biological replicates from 1 experiment (FIG. 14E).

FIGS. 15A-15F (related to FIGS. 9A-9F) graphically illustrate that adeno-associated viruses (AAVs) target necroptosis in tumor cells in situ, conferring tumor growth restriction that synergizes with immune checkpoint blockade to promote durable tumor clearance. FIG. 15A illustrates survival curves of mice bearing bilateral B16.F10-OVA tumors following administration of various death-inducing AAV2.5 constructs. N=10-12 mice per group. FIG. 15B illustrates B16.F10-OVA tumor growth curves following co-administration of intratumoral AAVs with IgG2a isotype control antibody. N=14-15 mice per group. FIG. 15C illustrates B16.F10-OVA tumor growth curves following co-administration of intratumoral AAVs with the immune checkpoint blockade reagent α-PD-1. N=13-16 mice per group. FIG. 15D illustrates survival curves of B16.F10-OVA tumor-bearing mice following co-administration of necroptosis-inducing RIPK3.coAAV with α-PD-1 in addition to CD8⁺ leukocyte depletion via antibody injection. Asterisks denote significant differences between corresponding IgG2b and α-CD8 treatment groups. N=3-8 mice per group. FIG. 15E illustrates survival curves B16.F10-OVA tumor-bearing Batf3^{-/-} or B6/J mice following co-administration of RIPK3.co AAV with α-PD-1. N=10-13 mice per group. FIG. 15F illustrates percentage of mice from panel 6C (or naive controls) that developed B16.F10-OVA tumors upon flank tumor re-challenge injection. N=8-10 mice per group. *p<0.05, ***p<0.001, ****p<0.0001. Black arrows indicate intratumoral AAV injections. Error bars represent SEM. Data are pooled from 2-4 independent experiments.

### DETAILED DESCRIPTION

The present disclosure is based on the inventors' investigation into signaling pathways that induce different forms of programmed cell death. As described in more detail below, the inventors developed fusion proteins that, upon expression in a cell, are constitutively active to induce various forms of programmed cell death, including apoptosis, necroptosis, and pyroptosis. This functionality occurs without the requirement for additional ligands or multimerization agents. Instead, the disclosed compositions incorporate functional death inducing domains fused with multimerization domains such that oligomers (such as dimers, trimers, and heptamers) can form automatically to elicit programmed death signaling cascades in the expressing cells.

To further advance the understanding of programmed cell death, the inventors demonstrated that ectopic introduction of necroptotic cells to the tumor microenvironment promotes Batf3⁺, DC1⁻, and CD8⁺ leukocyte-dependent anti-tumor immunity accompanied by increased tumor antigen loading by tumor-associated antigen presenting cells. Utilizing the fusion construct design, constitutively-active forms of the necroptosis-inducing enzyme RIPK3 effector domains were created and used to show that delivery of such fusion constructs, here via adeno-associated viruses (AAVs) comprising the encoding DNA, induces tumor cell necroptosis. Moreover, this approach synergized with immune checkpoint blockade to promote durable tumor clearance. These findings demonstrate that induced necroptosis is a beneficial proximal target in the initiation of tumor immunity.

In accordance with the foregoing, the disclosure provides methods and compositions to induce programmed cell death in cells. The methods and compositions are applicable, for example, to induction of programmed cell death in transformed or neoplastic cells, such as tumor cells. In some instances, these compositions and methods further provide the benefit of further inducing anti-tumor immunological responses that can be leveraged when combined with other forms of immunotherapy to provide a synergistic effect on the inhibition and destruction of the target cells. These benefits and others are provided by the inventions disclosed herein. Various aspects of the disclosure are now described in more detail.

Fusion Constructs

In one aspect, the disclosure provides a fusion protein comprising a death inducing domain and a multimerization domain.

The term “fusion protein” refers to an engineered polypeptide comprising discrete poly-amino acid domains. In some embodiments, the discrete domains can be disposed in any configuration together, whether contiguous (i.e., end to end within the overall protein molecule) or discontiguous (i.e., connected by an intervening linker segment within the overall protein molecule). The domains can be in any order within the fusion protein so long as the overall fusion protein retains the desired functionality. In some embodiments, a fusion protein comprises a death inducing domain and a multimerization domain. In some embodiments, the death inducing domain and the multimerization domain do not naturally occur together in a single protein. In some embodiments, the death inducing domain and the multimerization domain do not naturally occur together in the configuration as presented in the fusion protein.

Death Inducing Domain

The term “death inducing domain” refers to a domain that induces programmed cell death signaling under conditions in which the domain is functional. In some embodiments, the death inducing domain induces programmed cell death when complexed with one or more additional death inducing domains. For example, as described in more detail below, enzymes such as caspases, RIP kinases, and MLKL have functional death inducing domains. However, under endogenous conditions, these enzymes start as zymogen precursors that are activated by induced proximity. That is, when multiple copies of the proteins are brought into sufficient proximity, such as through the formation of multimers, the multiplicity of the death inducing domains initiate or substantially increase programmed cell death signaling. In some embodiments, the death inducing domain induces programmed cell death when complexed with one or more additional death inducing domains that are of the same type (e.g., a domain with at least about 80%, 85%, 90%, 95%, 98%, 99% or 100% sequence identity). Complexes (e.g., dimers, trimers, tetramers, pentamers, hexamers, heptamers, and the like) that include death inducing domains of the same type can be referred to generally as homo-multimers. However, in some embodiments, the death inducing domain of the disclosed fusion protein can induce programmed cell death when complexed with one or more additional death inducing domains of a different type (e.g., encoded by a different or unrelated gene). These complexes can be referred to as hetero-multimers.

The programmed cell death can be apoptosis, pyroptosis, or necroptosis. In some embodiments, the programmed cell death is apoptosis, which is a type of programmed cell death that typically occurs by normal, non-pathological processes. Apoptosis occurs following activation of certain caspase proteases. The apoptotic cells are typically characterized by membrane blebbing, but not swelling or pore formation. The clearance of apoptotic debris is nonimmunogenic and is often associated with tolerogenic signaling, resulting in immunomodulatory processes that include, e.g., the caspase-directed inactivation of immunostimulatory damage-associated molecular patterns (DAMPs), such as high-mobility group box-1 protein (HMGB1), as well as immunosuppressive functions of the Tyro3/Axl/Mertk receptor tyrosine kinases (TAM RTKs) that promote tissue repair phenotypes in phagocytes that have engulfed apoptotic debris. In some embodiments, the programmed cell death is pyroptosis, which is induced by a distinct signaling cascade, often associated with caspase-1 activation. Pyroptosis is typically induced by infection with intracellular pathogens and is likely to influence the antimicrobial immune response, including release of pro-inflammatory cytokines. Pyroptotic cells are characterized by cell swelling and lysis, with formation of membrane pores. Finally, in some embodiments, the programmed cell death is necroptosis, which results from yet another distinct signaling pathway that does not rely on caspases. Like pyroptosis, necroptotic cells are characterized by cell swelling and lysis, with formation of membrane pores. Unlike apoptosis, necroptotic cell death culminates in leakage of cell contents into the extracellular space and induction of immune responses.

A death inducing domain may comprise an effector domain. The term “effector domain” refers to a domain or subdomain of a reference protein that induces programmed cell death signaling under conditions in which the domain or subdomain is functional. In some embodiments, the death inducing domain comprises an effector domain selected from a caspase effector domain, a kinase effector domain, a MLKL effector domain, and a gasdermin effector domain. In some embodiments, the effector domain is functional to induce programmed cell death signaling in a cell when complexed with one or more additional effector domains, as described above. The reference protein from which the effector domain is derived can be from any mammal, such as a rodent (e.g., mouse or rat) or primate (e.g., human).

In some embodiments, a fusion protein comprises a death inducing domain and a multimerization domain, wherein the death inducing domain comprises an effector domain from a reference protein. In certain embodiments, the effector domain is selected from a caspase effector domain, a kinase effector domain, a MLKL effector domain, and a gasdermin effector domain. In certain embodiments, the fusion protein further comprises domains of the reference protein other than the effector domain, e.g., domains that facilitate protein interactions or signaling, as further described below.

Caspase-Derived Death Inducing Domains

The term “caspase effector domain” (also referred to as a “caspase protease domain”) refers to an effector domain that is derived from the caspase family of proteases. In some embodiments, the death inducing domain comprises a caspase effector domain. The caspase effector domain can be selected from a caspase 1 effector domain, a caspase 2 effector domain, a caspase 3 effector domain, a caspase 4 effector domain, a caspase 5 effector domain, a caspase 8 effector domain, a caspase 9 effector domain, a caspase 10 effector domain, and a caspase 11 effector domain.

Any protease domain from the above caspases is encompassed by the disclosure. Illustrative, non-limiting examples of various caspase effector domain sequences are described herein. The term “caspase effector domain” encompasses naturally occurring caspase effector domains (e.g., sequences indicated below) or functional variants thereof (either naturally occurring or engineered). Herein, a “functional variant” refers to a polypeptide or polypeptide domain that contains sequence variation relative to the parental sequence but retains the function, or a measurable portion of the function, of the parental polypeptide or polypeptide domain from which it is derived. For example, a functional variant of a death inducing domain, such as a caspase effector domain, fully or partially retains its function in inducing programmed cell death signaling under conditions in which the death inducing domain is functional. In some embodiments, a functional variant has at least about 80%, about 85%, about 90%, about 95%, about 98%, or about 99% sequence identity to the indicated reference sequence. In some embodiments, any sequence differences reflect conservative substitutions, as described below, or truncated sequences with end deletions.

For example, the present disclosure encompasses the caspase 1 effector domains represented by the sequences set forth in SEQ ID NOS:1-3 and 6, or functional variants thereof. SEQ ID NOS:1-3 are exemplary effector domains from murine caspase-1, whereas SEQ ID NO:6 is an exemplary human caspase-1 effector domain. Caspase 2 effector domains encompassed by the present disclosure include the illustrative, non-limiting example set forth in SEQ ID NO:152, or functional variants thereof. Caspase 3 effector domains encompassed by the present disclosure include the illustrative, non-limiting example set forth in SEQ ID NO:155, or functional variants thereof. Caspase 4 effector domains encompassed by the present disclosure include the illustrative, non-limiting example set forth in SEQ ID NO:9 (a representative human caspase-4 protease domain), or functional variants thereof. Caspase 5 effector domains encompassed by the present disclosure include the illustrative, non-limiting example set forth in SEQ ID NO:12 (a representative human caspase-5 protease domain), or functional variants thereof. Caspase 8 effector domains encompassed by the present disclosure include the illustrative, non-limiting example set forth in SEQ ID NO:15 (a representative human caspase-8 protease domain), or functional variants thereof. Caspase 9 effector domains encompassed by the present disclosure include the illustrative, non-limiting example set forth in SEQ ID NO:18 (a representative human caspase-9 protease domain), or functional variants thereof. Caspase 10 effector domains encompassed by the present disclosure include the illustrative, non-limiting example set forth in SEQ ID NO:156, or functional variants thereof. Caspase 11 effector domains encompassed by the present disclosure include the illustrative, non-limiting example set forth in SEQ ID NO:159, or functional variants thereof.

In some embodiments, a fusion protein comprises (i) a death inducing domain comprising a caspase effector domain and (ii) a multimerization domain. In such embodiments, the fusion protein may further comprise a caspase prodomain. Most naturally occurring caspases contain an additional prodomain that is near or adjoins the N-terminal end of the protease domain. In the disclosed fusion proteins, the optional prodomain can be derived from the same caspase as the caspase effector (i.e., protease) domain. For example, both the caspase effector domain and prodomain can be from caspase-1. In other embodiments, the caspase effector domain and prodomain can be from a homologous or different caspase (e.g., the prodomain from caspase-1 and the effector domain from caspase-2), so long as the effector domain retains its intended function as described above. To illustrate, an exemplary murine caspase 1 prodomain is set forth in SEQ ID NO:4. An exemplary combination of a murine caspase-1 protease domain (SEQ ID NO:1) and a murine prodomain (SEQ ID NO:4) is set forth in SEQ ID NO:5. An exemplary human caspase-1 prodomain is set forth in SEQ ID NO:7. An exemplary combination of human caspase-1 protease domain (SEQ ID NO:6) and a human prodomain (SEQ ID NO:7) is set forth in SEQ ID NO:8. An exemplary human caspase-2 prodomain is set forth in SEQ ID NO:153. An exemplary combination of human caspase-2 protease domain (SEQ ID NO:152) and a human prodomain (SEQ ID NO:153) is set forth in SEQ ID NO:154. An exemplary human caspase 4 prodomain is set forth in SEQ ID NO:10. An exemplary combination of human caspase-4 protease domain (SEQ ID NO:9) and a murine prodomain (SEQ ID NO:10) is set forth in SEQ ID NO:11. An exemplary human caspase-5 prodomain is set forth in SEQ ID NO:13. An exemplary combination of human caspase-5 protease domain (SEQ ID NO:12) and a murine prodomain (SEQ ID NO:13) is set forth in SEQ ID NO:14. An exemplary human caspase-8 prodomain is set forth in SEQ ID NO:16. An exemplary combination of human caspase-8 protease domain (SEQ ID NO:15) and a murine prodomain (SEQ ID NO:16) is set forth in SEQ ID NO:17. An exemplary human caspase-9 prodomain is set forth in SEQ ID NO:19. An exemplary combination of human caspase-9 protease domain (SEQ ID NO:18) and a murine prodomain (SEQ ID NO:19) is set forth in SEQ ID NO:20. An exemplary human caspase-10 prodomain is set forth in SEQ ID NO:157. An exemplary combination of human caspase-10 protease domain (SEQ ID NO:156) and a human prodomain (SEQ ID NO:157) is set forth in SEQ ID NO:158. An exemplary murine caspase-11 prodomain is set forth in SEQ ID NO:160. An exemplary combination of murine caspase-11 protease domain (SEQ ID NO:159) and a murine prodomain (SEQ ID NO:160) is set forth in SEQ ID NO:161.

Thus, in some embodiments the death inducing domain comprises an amino acid sequence as set forth in any one of SEQ ID NOS:1-3, 5, 6, 8, 9, 11, 12, 14, 15, 17, 18, and 20, or a functional variant thereof comprising an amino acid sequence with at least about 80%, about 85%, about 90%, about 95%, about 98%, or about 99% sequence identity thereto. In some embodiments, the functional variants are conservatively modified variants.

Kinase-Derived Death Inducing Domains

The term “kinase effector domain” (also referred to as a “kinase domain”) refers to an effector domain that is derived from a member of a kinase family. In some embodiments, the death inducing domain comprises a kinase effector domain. In some embodiments, the kinase effector domain comprises a receptor-interacting serine/threonine-protein kinase (RIPK) kinase effector domain. In some embodiments, the kinase effector domain comprises any one of a RIPK1 kinase effector domain, a RIPK2 kinase effector domain, and a RIPK3 kinase effector domain.

Any kinase domain from the above RIPK enzymes is encompassed by the disclosure. Illustrative, non-limiting examples of various RIPK effector domain sequences are described herein. The term “kinase effector domain” encompasses naturally occurring kinase effector domains (e.g., RIP kinase effector domains such as the sequences indicated below) or functional variants thereof (either naturally occurring or engineered), as defined and exemplified herein.

For example, the present disclosure encompasses the RIPK3 kinase effector domains represented by the sequences set forth in SEQ ID NOS:21 and 25, or functional variants thereof. SEQ ID NO:21 is an exemplary murine RIPK3 kinase effector domain, whereas SEQ ID NO:25 is an exemplary human RIPK3 kinase effector domain. RIPK1 kinase effector domains encompassed by the present disclosure include the illustrative, non-limiting example set forth in SEQ ID NO:28 (a human RIPK1 kinase effector domain), or functional variants thereof. RIPK2 kinase effector domains encompassed by the present disclosure include the illustrative, non-limiting example set forth in SEQ ID NO:31 (a human RIPK2 kinase effector domain), or functional variants thereof.

In some embodiments, a fusion protein comprises (i) a death inducing domain comprising a RIP kinase effector domain (e.g., a RIPK1, RIPK2, or RIPK3 kinase effector domain) and (ii) a multimerization domain. In such embodiments, the fusion protein may further comprise a further RIPK domain, such as an interaction domain or a RIP homotypic interaction motif (RHIM) domain from the same or different RIPK as the RIP kinase effector domain. The RHIM (or interaction) domain can be disposed contiguously with the kinase effector domain or in some embodiments the RHIM domain is separated from the kinase effector domain by intervening sequence. In some embodiments, the intervening sequence is also derived from the parental RIPK protein. Exemplary RHIM or interaction domains and combinations with RIP kinase effector domains are provided. However it will be appreciated that RHIM domains, interaction domains, and RIP kinase effector domains, or any intervening sequence, need not be derived from the same parental RIPK sequence for purposes of a functional death inducing domain. An exemplary murine RIPK3 RHIM domain is represented by the sequence set forth in SEQ ID NO:22. An exemplary combination of a murine RIPK3 kinase effector domain (SEQ ID NO:21) and a murine RHIM domain (SEQ ID NO:22) is set forth in SEQ ID NO:23, which also includes intervening sequence. As an alternative embodiment, SEQ ID NO:24 represents a truncated version of SEQ ID NO:23, where the C-terminal end with the RHIM domain is excised. An exemplary human RIPK3 RHIM domain is represented by the sequence set forth in SEQ ID NO:26. An exemplary combination of a human RIPK3 kinase effector domain (SEQ ID NO:25) and a human RIPK3 RHIM domain (SEQ ID NO:26) is set forth in SEQ ID NO:27, which also includes intervening sequence. An exemplary human RIPK1 RHIM domain is represented by the sequence set forth in SEQ ID NO:29. An exemplary combination of a human RIPK1 kinase effector domain (SEQ ID NO:28) and a human RIPK1 RHIM domain (SEQ ID NO:29) is set forth in SEQ ID NO:30, which also includes intervening sequence. An exemplary human RIPK2 RHIM domain is represented by the sequence set forth in SEQ ID NO:32. An exemplary combination of a human RIPK2 kinase effector domain (SEQ ID NO:31) and a human RIPK2 interaction domain (SEQ ID NO:32) is set forth in SEQ ID NO:33, which also includes intervening sequence.

Thus, in some embodiments the death inducing domain comprises an amino acid sequence as set forth in one of SEQ ID NOS:21, 23-25, 27, 28, 30, 31, and 33, or a functional variant thereof comprising an amino acid sequence with at least about 80%, about 85%, about 90%, about 95%, about 98%, or about 99% sequence identity thereto. In some embodiments, the functional variants are conservatively modified variants.

MLKL-Derived Death Inducing Domains

The term “MLKL effector domain” (also referred to as an “MLKL pore-forming domain”) refers to an effector domain that is derived from the Mixed Lineage Kinase Domain Like Pseudokinase (MLKL). In some embodiments, a death inducing domain comprises an MLKL effector domain. Any MLKL effector domain is encompassed by this disclosure. The term “MLKL effector domain” encompasses naturally occurring MLKL effector domains (e.g., MLKL effector domains having the sequences indicated below) or functional variants thereof (either naturally occurring or engineered), as defined and exemplified herein. An illustrative, non-limiting human MLKL effector domain is represented by the sequence set forth in SEQ ID NO:34 or a functional variant thereof.

In some embodiments, a fusion protein comprises (i) a death inducing domain comprising an MLKL effector domain and (ii) a multimerization domain. In such embodiments, the fusion protein may further comprise an MLKL pseudokinase domain, or a portion thereof, from the same or different MLKL as the MLKL effector domain. Exemplary MLKL pseudokinase domains are represented by the sequence set forth in SEQ ID NO: 35, or a variant thereof. An illustrative combination of a human MLKL effector domain (SEQ ID NO:34) and an MLKL pseudokinase domain (SEQ ID NO:35) is set forth in SEQ ID NO:36.

Thus, in some embodiments the death inducing domain comprises an amino acid sequence as set forth in any one of SEQ ID NOS:34 and 36 or a functional variant thereof comprising an amino acid sequence with at least about 80%, about 85%, about 90%, about 95%, about 98%, or about 99% sequence identity thereto. In some embodiments, the functional variants are conservatively modified variants.

GSDMD-Derived Death Inducing Domains

The term “gasdermin effector domain,” “gasdermin D effector domain,” “GSDMD effector domain,” or “GSDMD pore-forming domain” refers to an effector domain that is derived from gasdermin D (GSDMD). In some embodiments, a death inducing domain comprises a GSDMD effector domain. Any GSDMD effector domain is encompassed by this disclosure. The term “GSDMD effector domain” (and interchangeable terms) encompasses naturally occurring GSDMD effector domains (e.g., GSDMD effector domains having the sequences indicated below) or functional variants thereof (either naturally occurring or engineered), as defined and exemplified herein. An illustrative, non-limiting GSDMD effector domain is represented by the sequence set forth in SEQ ID NO:37 (which is a human GSDMD effector domain) or a functional variant thereof as defined.

In some embodiments, a fusion protein comprises (i) a death inducing domain comprising a GSDMD effector domain and (ii) a multimerization domain. In such embodiments, the fusion protein may further comprise a GSDMD auto-inhibitory domain, or a portion thereof, from the same or different GSDMD as the GSDMD effector domain. Exemplary GSDMD auto-inhibitory domains are represented by the sequence set forth in SEQ ID NO: 38, or a functional variant thereof. An illustrative combination of a GSDMD effector domain (SEQ ID NO:37) and a GSDMD auto-inhibitory domain (SEQ ID NO:38) is set forth in SEQ ID NO:39.

Thus, in some embodiments the death inducing domain comprises an amino acid sequence as set forth in one of SEQ ID NOS:37 and 39 or a functional variant thereof comprising an amino acid sequence with at least about 80%, about 85%, about 90%, about 95%, about 98%, or about 99% sequence identity thereto. In some embodiments, the functional variants are conservatively modified variants.

Multimerization Domain

As indicated above, any embodiment of a fusion protein also comprises a multimerization domain. As used herein, the term “multimerization domain” refers to a domain that physically associates with other such domains with sufficient affinity such that the domains are held in proximity to one another. In the context of the disclosed fusion proteins, the death inducing domains are held in sufficient proximity to one another such that they can function to induce programmed cell death signaling in a cell. In some embodiments, the multimerization domains homo-multimerize, meaning they form complexes with identical multimerization domains, or multimerization domains with minor variations (i.e., variants with at least about 80%, about 85%, about 90%, about 95%, about 98%, or about 99% sequence identity).

The disclosed multimerization domains can function to produce dimers, trimers, tetramers, pentamers, heptamers, or octamers of the fusion proteins in which they occur. In some embodiments, these multimers are homo-dimers, homo-trimers, homo-tetramers, homo-pentamers, homo-hexamers, homo-heptamers, or homo-octamers, as defined above, with respect to the sequence and structure of the multimerization domain (but not with respect to other sequences in the fusion protein). Accordingly, the fusion protein multimerization domain may comprise a dimerization domain, a trimerization domain, a tetramerization domain, a pentamerization domain, a hexamerization domain, a heptamerization domain, or an octamerization domain. In some embodiments, the fusion protein comprises two or more multimerization domains, which can include two or more of the same multimerization domain or different multimerization domains in the same fusion protein. Thus, in some embodiments, the fusion protein can comprise two or more of a trimerization domain, a tetramerization domain, a pentamerization domain, a hexamerization domain, a heptamerization domain, or an octamerization domain, in any combination.

Representative, non-limiting multimerization domains encompassed by the present disclosure and useful in the disclosed fusion proteins include engineered multimerization domains described in published International Application No.: WO/2017/173356, and also described in Boyken, S. E., et al., De novo design of protein homo-oligomers with modular hydrogen-bond network-mediated specificity. Science 352, 680-687 (2016), both of which are incorporated herein by reference. Briefly, the exemplary multimerization domains are engineered to form two concentric rings of helices. The helices contain polar groups to provide the opportunity for hydrogen bonding. The helical structure permits repetitive presentation of side chains that permit hydrogen bonding with partner helices. With activity analogous to the Watson-Crick base pairing in nucleic acids, the pair poly-amino acid helices interact with one or more pairs of concentric helical rings from other domains. The result is a hydrogen bond network along the interface of the helices from the individual domains. The two-ring concentric structure of each domain has been shown to maximize the hydrogen bonding, which functionally confers strength and specificity of the multimerization. The wide range of hydrogen-bond networks permitted by this design allows for formation of a variety of geometries and hypercoiling topologies, such as pairs (for dimers), triangles (for trimers), squares (for tetramers), and the like.

In some embodiments, the multimerization domain comprises one of: 2LHC2-23 (SEQ ID NO:40), 5L6HC3-1 (SEQ ID NO:43), 2L6HC3-9 (SEQ ID NO:148), 2L6HC3-13 (SEQ ID NO:147), 6H8 (SEQ ID NO:149), and 7H3 (SEQ ID NO:44). In some embodiments, the multimerization domain comprises an amino acid sequence as set forth in any one of SEQ ID NOS:40-44, 58-135, and 147-149, or a functional variant thereof comprising an amino acid sequence with at least with at least about 80%, about 85%, about 90%, about 95%, about 98%, or about 99% sequence identity thereto. In some embodiments, the differences in the variant sequence are conservative modifications or variations. It is noted that SEQ ID NOS:40-42 are the sequences of exemplary dimerization domains, SEQ ID NO:43 is the sequence of an exemplary trimerization domain, and SEQ ID NO:44 is the sequence of an exemplary heptamerization domain.

The present disclosure encompasses all combinations of disclosed death domains, e.g., the caspase, RIPK, MLKL, GSDMD effector domains described above) and multimerization domain types (e.g., a trimerization domain, a tetramerization domain, a pentamerization domain, a hexamerization domain, a heptamerization domain, or an octamerization domain) as any multimer (e.g., dimer, trimer, tetramer, pentamer, hexamer, heptamer, octamer, etc.) of any of the disclosed death domains will exhibit functional programmed cell death signaling. In some embodiments, the fusion protein comprises a caspase domain with a dimerization domain. In some embodiments, the fusion protein comprises a RIPK domain with a higher order multimerization domain, e.g., a tetramerization domain, a pentamerization domain, a hexamerization domain, a heptamerization domain, or an octamerization domain. In some embodiments, the fusion protein comprises a RIPK domain with a trimerization domain.

Exemplary, non-limiting fusion proteins of the disclosure that combine a death domain with a multimerization domain include those represented by the amino acid sequences set forth in SEQ ID NOS:45 and 46 (incorporating a murine caspase 1 effector domain and a dimerization domain, with and without a prodomain), SEQ ID NO:47 (incorporating a murine caspase 1 effector domain and a heptamerization domain 47), SEQ ID NOS:48 and 49 (incorporating a human caspase-8 effector domain with a dimerization domain, with and without a prodomain), SEQ ID NOS:50 and 51 (incorporating a human caspase-9 effector domain with a dimerization domain, with and without a prodomain), SEQ ID NOS:52 and 53 (incorporating a human RIPK3 domain with a trimerization domain, with and without a RHIM domain), SEQ ID NOS:54 and 55 (incorporating an MLKL effector domain with a trimerization domain, with and without a pseudokinase domain), SEQ ID NOS:56 and 57 (incorporating a GSDMD effector domain with a heptamerization domain, with and without an auto-inhibitory domain), and any functional (e.g., conservative) variant thereof that comprises a sequence with at least about 80%, about 85%, about 90%, about 95%, about 98%, or about 99% sequence identity to any of the foregoing sequences.

Linker

As indicated above, the death inducing domain and the multimerization domain can be contiguous within the fusion protein, in any order from the N-terminal side to the C-terminal side. In other embodiments, the death inducing domain and the multimerization domain are not contiguous, but instead are separated by a linker domain. A “linker domain” refers to any amino acid sequence that serves as intervening spacer between two domains. The physical separation provided by the linker can be useful to allow each of the death inducing domain and the multimerization domain to simultaneously maintain their respective functions while avoiding or decreasing steric hindrance between them. The length of the linker is not critical and is preferably of a length that avoids or decreases steric hindrance between the death inducing domain and the multimerization domain. Thus, the linker can be a peptide with at least a single amino acid, such as 2, 3, 4, 5, 6, 7, 8, 9, 10 or more amino acids. However, it will be understood that the linker can be substantially longer, ranging from 10 to 100 or even more amino acids long. The linker may be flexible to facilitate activity of each domain in the fusion protein. Furthermore, in some embodiments, the linker domain is not reactive. For example, the linker domain does not substantially interact with cytosolic components. In some embodiments, the linker can comprise one or more alanine residues, serine residues, glycine residues, or a combination thereof. Exemplary linkers used in the disclosed investigations include the sequences set forth in SEQ ID NOS:150 and 151, which are encompassed by the present disclosure.

In some embodiments, the linker is directly linked to one of the death inducing domain and the multimerization domain at its N-terminal end and to the other of the death inducing domain and the multimerization domain at its C-terminal end.

In certain embodiments, the linker is functionally incorporated into the fusion protein by using recombinant techniques to incorporate a nucleic acid sequence encoding the linker between nucleic acid sequences encoding the death inducing domain and the multimerization domain. The resulting nucleic acid construct may then be expressed, e.g., in a cell.

Nucleic Acids and Related Constructs

In another aspect, the disclosure provides a nucleic acid molecule encoding any of the fusion proteins described herein. For example, a person of ordinary skill in the art can use the genetic code to determine nucleic acid sequences that can encode fusion proteins comprising a death inducing domain, a multimerization domain, and an optional linker domain, based on the above disclosures. In some embodiments, the nucleic acid further comprises a promoter sequence operatively linked to the sequence encoding the fusion protein. The term “promoter” refers to a regulatory nucleotide sequence that can activate transcription (expression) of a gene and/or splice variant isoforms thereof. A promoter is typically located upstream of a gene, but can be located at other regions proximal to the gene, or even within the gene. The promoter typically contains binding sites for RNA polymerase and one or more transcription factors, which participate in the assembly of the transcriptional complex. As used herein, the term “operatively linked” indicates that the promoter and the encoding nucleic acid are configured and positioned relative to each other a manner such that the promoter can activate transcription of the encoding nucleic acid by the transcriptional machinery of the cell. The promoter can be constitutive or inducible. Constitutive promoters can be determined based on the character of the target cell and the particular transcription factors available in the cytosol. A person of ordinary skill in the art can select an appropriate promoter based on the intended person, as various promoters are known and commonly used in the art.

In some embodiments, the disclosure provides a vector comprising the nucleic acid described above. The vector can be any construct that facilitates the delivery of the nucleic acid to the target cell and/or expression of the nucleic acid within the cell. The vectors can be viral vectors, circular nucleic acid constructs (e.g., plasmids), or nanoparticles.

Various viral vectors are known in the art and are encompassed by the present disclosure. See, e.g., Machida, C. A. (ed.), Viral Vectors for Gene Therapy: Methods and Protocols, Humana Press, Totowa, N.J. (2003); Muzyczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno associated virus (AAV) vector, an adenovirus vector, a retrovirus vector, or a lentivirus vector. A specific embodiment of an AAV vector includes the AAV2.5 serotype.

Cells

In another aspect, the disclosure provides a cell comprising the nucleic acid encoding any fusion protein as described herein. In some embodiments, the cell comprises a vector, wherein the vector comprises the nucleic acid encoding any fusion protein as described herein. The cell is capable of expressing the fusion protein from the nucleic acid. For example, the nucleic acid and/or vector can be configured for expression of the fusion protein from the encoding nucleic acid within the cell. A promoter operatively linked to the nucleic acid can be appropriately configured to allow binding of the cell's RNA polymerase and one or more transcription factors to permit assembly of the transcriptional complex.

The disclosure encompasses any type of cell for this aspect. Exemplary embodiments include tumor cells of any kind (nonlimiting examples of tumor cells are provided herein and are encompassed by this aspect), a tumor-associated stromal cell, or other cell. The “other cell” can include non-cancerous cells that are useful with the induction of programmed cell death, such as in applications for research or immunological manipulations. For example, as described in more detail below, fibroblasts and cancer cells (e.g., such as derived from melanoma and lung adenocarcinoma) were transformed to contain nucleic acids encoding fusions proteins described herein. The cells were operable to induce immunological responses against tumors to which the necroptotic cells were administered.

The disclosure also encompasses therapeutic compositions comprising the disclosed cells. The compositions can comprise appropriate culture media. In some embodiments, the compositions comprise appropriate carriers for intra-tumoral administrations.

Methods

In another aspect, the disclosure provides a method of inducing programmed cell death in a cell. The method comprises providing the cell with multiple copies of the fusion protein as described above. Providing multiple copies can comprise administering, contacting, or otherwise delivering the multiple copies of the fusion protein to the cell. The fusion proteins can be readily and appropriately formulated accordingly to common practice in the art for delivery to the cell, whether in vitro or in vivo. In other embodiments, providing multiple copies comprises causing expression of the fusion protein in the cell from a nucleic acid as described herein. Thus, the step of providing multiple copies of the fusion protein comprises administering, contacting, or otherwise delivering the nucleic acid, or a vector comprising the nucleic acid, to the cell and permitting or inducing or allowing the expression of the encoded fusion protein within the cell. Strategies for transfecting a cell with a nucleic acid or vector containing the nucleic acids are known and can be routinely applied in the present context. Such transfection techniques can include, for example, electroporation, administering calcium phosphate, and the like. The approach can be dependent on the vector used. For example, AAV vectors can be used that specifically express the fusion protein in the cell of interest. As described in more detail below, fusion proteins including a 2L6HC3-9 homotrimerizing domain and an RIPK3 effector domain were expressed in cancer cells using an AAV vector under the control of synthetic MND promoter. In a similar approach, the fusion protein was also expressed in B16.F10 melanoma cells using a hybrid AAV2.5 serotype vector (Bowles D. E., et al. (2012). Phase 1 gene therapy for Duchenne muscular dystrophy using a translational optimized AAV vector. Mol Ther. 20, 443-455, incorporated herein by reference in its entirety).

As described in more detail above and below, the programmed cell death induced in the cell can be apoptosis, pyroptosis, or necroptosis, depending on the selection of the specific death inducing domain. For example, in embodiments where the death inducing domain is a caspase effector domain selected from: a caspase 2 effector domain, a caspase 3 effector domain, a caspase 8 effector domain, a caspase 9 effector domain, and a caspase 10 effector domain, the programmed cell death induced in the cell is apoptosis. Alternatively, in embodiments where the death inducing domain is a caspase effector domain selected from: a caspase 1 effector domain, a caspase 4 effector domain, a caspase 5 effector domain, a caspase 9 effector domain, and a caspase 11 effector domain, the programmed cell death induced in the cell is pyroptosis. Furthermore, in embodiments where the death inducing domain is a gasdermin effector domain, the programmed cell death induced in the cell is pyroptosis. Still further, in embodiments where the death inducing domain is selected from: a RIP1 kinase effector domain, a RIPK2 kinase effector domain, a RIPK3 kinase effector domain, and a MLKL effector domain, the programmed cell death induced in the cell is necroptosis.

The cell can be any target cells where induction of programmed cell death is desired. The cells are typically eukaryotic, for example, mammalian in origin. The method is particularly useful for target cells that are tumor cells. As used herein, tumor cells refers to cancer cells generally, which are transformed, neoplastic cells that have abnormal growth and division characteristics.

The cancer cells can be (or be derived from) a solid tumor cancer.

The solid tumor can be derived (i.e., originate) from any tissue. Illustrative, non-limiting examples of solid tumor cancer include pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, and soft tissue sarcoma.

In some embodiments, the method can be performed such that the programmed cell death is induced in a cell in vitro. Typically, one or more cells are maintained in a suitable culture medium. The multiple copies of the fusion protein (or nucleic acid encoding the same) are administered to the cells in the culture medium. Strategies for administering the cells with pre-expressed fusion proteins are known and include techniques such as use of electroporation and liposomal or other nanoparticle-based delivery techniques. As indicated above, strategies for transfecting a cell with nucleic acids that allow for transgenic expression of the fusion protein are also known. Such strategies also include electroporation. In other embodiments, viral vector systems are used as described herein. An exemplary viral vector is AAV, such as AAV 2.5, which can be used to deliver the nucleic acid under a control of a promoter that permits specific expression in the target cell.

In other embodiments, the programmed cell death is induced in the cell in vivo in a subject with a tumor or cancer. Accordingly, in another aspect the disclosure provides a method of treating cancer in a subject. The method comprises administering to the subject a therapeutically effective amount of the fusion protein disclosed herein.

As used herein, the term “treat” refers to medical management of a disease, disorder, or condition (e.g., cancer, as described above) of a subject (e.g., a human or non-human mammal, such as another primate, horse, dog, mouse, rat, guinea pig, rabbit, and the like). Treatment can encompasses any indicia of success in the treatment or amelioration of a disease or condition (e.g., a cancer), including any parameter such as abatement, remission, diminishing of symptoms or making the disease or condition more tolerable to the patient, slowing in the rate of degeneration or decline, or making the degeneration less debilitating. Specifically in the context of cancer, the term treat can encompass slowing or inhibiting the rate of cancer growth, or reducing the likelihood of recurrence, compared to not having the treatment. In some embodiments, the treatment encompasses resulting in some detectable degree of cancer cell death in the patient. The treatment or amelioration of symptoms can be based on objective or subjective parameters, including the results of an examination by a physician. Accordingly, the term “treating” includes the administration of the compositions of the present disclosure to alleviate, or to arrest or inhibit development of the symptoms or conditions associated with disease or condition (e.g., cancer). The term “therapeutic effect” refers to the amelioration, reduction, or elimination of the disease or condition, symptoms of the disease or condition, or side effects of the disease or condition in the subject. The term “therapeutically effective” refers to an amount of the composition that results in a therapeutic effect and can be readily determined.

As indicated above, administering to the subject a therapeutically effective amount of the fusion protein can comprise administering a therapeutically effective amount of a nucleic acid encoding the fusion protein to cause the expression of the fusion protein in the target cell. The nucleic acid is typically configured for expression in the target cell, i.e., being operatively linked to an appropriate promoter. The nucleic acid can be integrated into a vector configured to deliver and/or express the nucleic acid in a cancer cell or a non-cancer cell, such as a stromal cell associated with a tumor, as described in more detail above. The vector can be rationally designed to target delivery of the nucleic acid to (and cause expression of the encoded fusion protein in) the cancer cell or non-cancer cell such that minimal off target expression of the fusion protein occurs. The targeting is rationally designed based on the character of the target cell, which would be known to the practitioner.

In some embodiments, the method of treatment is combined or coordinated with other cancer therapeutic strategies. Any other cancer therapeutic strategy is contemplated in this combinatorial aspect. In some embodiments, the other cancer strategy is a cancer immunotherapy that utilizes immunomodulatory compositions (e.g., antibodies, immune cells, cytokines, etc.), which may boost the subject's own immune response against the cancer target. Such immuno therapies include adoptive immune cell therapies, including CAR T-cells, immune checkpoint inhibitor therapies, cancer vaccines, and the like.

In one embodiment, the disclosed method is combined or coordinated with administration of a PD-1 pathway inhibitor. A “PD-1 pathway inhibitor” refers to an agent that inhibits the PD-1/PD-L1 signaling pathway to a measurable extent. In some embodiments, the PD-1 pathway inhibitor acts by binding to PD-1 and/or PD-L1. In some embodiments, the PD-1 pathway inhibitor also binds to PD-L2. In some embodiments, a PD-1 pathway inhibitor blocks (completely or partially) binding of PD-1 to PD-L1 and optionally PD-L2. Nonlimiting exemplary PD-1 pathway inhibitors include PD-1 antagonists, such as antibodies that bind to PD-1, and PD-L1 antagonists, such as antibodies that bind to PD-L1. It is noted that the term “antibody” is used herein in the broadest sense and encompasses various antibody structures, including but not limited to monoclonal and polyclonal antibodies; monovalent, bivalent and multivalent antibodies; multispecific antibodies (e.g., bispecific antibodies); and antibody fragments so long as they exhibit the desired antigen-binding activity. Examples of such antibody fragments include, but are not limited to, Fv, single-chain Fv (scFv), Fab, Fab′, and (Fab′)₂ fragments. Nonlimiting exemplary of antibodies that bind to PD-1 include nivolumab and pembrolizumab; and Nonlimiting exemplary of antibodies that bind to PD-L1 include atezolizumab, durvalumab and avelumab.

As described in more detail below, the inventors demonstrated that certain forms of cell programmed death, namely necroptosis, induced immune activation directed to the cancer cells. The effect was demonstrated by administration of necroptotic cells to the tumor microenvironment as well as inducing expression of necroptosis-inducing fusion protein constructs directly in the tumor cells using a viral vector system.

Accordingly, in another aspect, the disclosure provides a method of inhibiting growth of tumor cells in a subject. The method comprises modifying the tumor microenvironment of the tumor cells to contain one or more necroptotic cells. The one or more necroptotic cells comprise multimers of engineered proteins that comprise a necroptotic death inducing domain.

The engineered proteins contain a non-naturally occurring combination of a death (specifically necroptosis) inducing domain with a multimerization domain. In this context, the multimerization domain can be constitutively active, i.e., multimerizing without addition or presence of an additional interaction ligand or partner to facilitate multimerization. However, the multimerization domain need not be constitutively active, but can be rationally designed to be inducible upon administration of a binding partner. An example of an inducible multimerization strategy is described in more detail below.

As indicated above the presence of one or more necroptotic cells within the tumor microenvironment can promote an antitumor immune response in the subject. In some embodiments, this modification of the tumor microenvironment comprises administering necroptotic cells to the microenvironment. In some embodiments, the cells have been induced to undergo necroptosis in vitro prior to administering the cells to the microenvironment. In some embodiments, the necroptotic cells are administered prior to their death. However, some but not all of the necroptotic cells may be dead prior to the administration. For example, the step of modifying the tumor microenvironment of the tumor cells to contain necroptotic cells can comprise inducing multimerization of two or more engineered proteins comprising a necroptotic death inducing domain within one or more cells in vitro, and administering the one or more cells to the tumor microenvironment prior to the death of the one or more cells. Administration of cells can include any acceptable technique, including intratumoral administration. Intratumoral injection includes injection directly to the tumor microenvironment. In some embodiments, the one or more cells are tumor cells that are matched (autologous) to the tumor cells in the tumor microenvironment. For example, the one or more cells can be initially obtained from the tumor microenvironment prior to the step of inducing multimerization of the two or more proteins. In other embodiments, the cells are obtained from the same source tissue as the tumor microenvironment. (For example, if the tumor is a breast tumor, then cells from breast tissue may be obtained.) In some embodiments, the cells are autologous (i.e., initially obtained from the same subject).

To illustrate, in some embodiments cells can be obtained from a subject diagnosed with a tumor. The cells can be obtained from the same tissue from which the tumor is derived. Alternatively, the cells can be tumor cells from (e.g., from a biopsy) or from stromal cells associated with the tumor. The obtained cells can be cultured in vitro during which phase they are administered an effective amount of fusion protein or are transfected with the nucleic encoding the fusion protein. An exemplary transfection approach is described in more detail below, which incorporates the nucleic acid into an AAV (e.g., AAV 2.5) vector wherein the nucleic acid is under the control of a promoter that allows expression in the cell. Once necroptosis is induced, the cells are injected into the tumor microenvironment in an appropriate carrier.

In some embodiments, the engineered proteins are or comprise a fusion protein as disclosed above that comprises both a multimerization domain and a necroptosis death inducing domain (e.g., a RIPK1 kinase effector domain, a RIPK2 kinase effector domain, a RIPK3 kinase effector domain, and a MLKL effector domain). Exemplary engineered proteins encompassed by this embodiment include SEQ ID NOS:52 and 53 (incorporating a human RIPK3 domain with a trimerization domain, with and without a RHIM domain), SEQ ID NOS:54 and 55 (incorporating an MLKL effector domain with a trimerization domain, with and without a pseudokinase domain), and a functional variant comprising an amino acid sequence with at least about 80%, about 85%, about 90%, about 95%, about 98%, or about 99% sequence identity thereto. In some embodiments, the sequence variants are conservative variants.

In some embodiments, the engineered proteins are or comprise a fusion protein as disclosed above that comprises both a multimerization domain and a necroptosis death inducing domain (e.g., a RIPK1 kinase effector domain, a RIPK2 kinase effector domain, a RIPK3 kinase effector domain, and a MLKL effector domain). Exemplary engineered proteins encompassed by this embodiment include SEQ ID NOS:52 and 53 (incorporating a human RIPK3 domain with a trimerization domain, with and without a RHIM domain), SEQ ID NOS:54 and 55 (incorporating an MLKL effector domain with a trimerization domain, with and without a pseudokinase domain), and an amino acid sequence with at least about 80%, about 85%, about 90%, about 95%, about 98%, or about 99% sequence identity thereto. In some embodiments, the sequence variants are conservative variants.

The necroptosis can be induced in the one or more cells by contacting the one or more cells with a nucleic acid or a vector comprising the nucleic acid, wherein the nucleic acid comprises a sequence encoding one of a RIPK1 kinase effector domain, a RIPK2 kinase effector domain, a RIPK3 kinase effector domain, and a MLKL effector domain. The nucleic acids and vectors are described in more detail above and are specifically encompassed by this aspect of the disclosure. Modifying the tumor microenvironment of the tumor cells can comprise contacting the one or more cells with the nucleic acid or vector in vitro and subsequently administering the one or more cells to the tumor microenvironment. In some embodiments, the method also comprises actively obtaining the one or more cells from the tumor microenvironment prior to the step of contacting the one or more cells with the nucleic acid or vector.

As an alternative to inducing necroptosis in select cells in vitro and then administering the necroptotic cells to the tumor microenvironment, in other embodiments, the step of modifying the tumor microenvironment of the tumor cells comprises contacting the one or more cells with the nucleic acid or vector in vivo. Thus, in some embodiments, the one or more cells referred to above are tumor cells in the tumor microenvironment. However, the one or more cells can also be or comprise stromal cells associated with the tumor in the tumor microenvironment.

For example, in some embodiments, the cells are administered nucleic acids encoding a fusion protein that comprises a RIPK3 effector domain with an amino acid sequence as set forth in SEQ ID NO:21 or 25, or a functional variant thereof comprising an amino acid sequence with at least about 80%, about 85%, about 90%, about 95%, about 98%, or about 99% sequence identity thereto. In some embodiments, the multimerization domain in the fusion protein has a sequence set forth in one of SEQ ID NOS:40-44, 58-135, and 147-149. In some embodiments, the multimerization domain in the fusion protein is a trimerizing domain or higher level multimerization domain. In some embodiments, the fusion protein comprises the trimerizing domain SEQ ID NO:43. In some embodiments, the fusion protein comprises the trimerizing domain SEQ ID NO:147. In other embodiments, the fusion protein comprises the heptamerizing domain SEQ ID NO:44. In one embodiment, the fusion protein comprises an amino acid sequence as set forth in SEQ ID NO:52 or 53, or a functional variant thereof comprising an amino acid sequence with at least about 80%, about 85%, about 90%, about 95%, about 98%, or about 99% sequence identity thereto. In one embodiment, the cell is administered the nucleic acids in vitro. In an alternative embodiment, the cell is administered the nucleic acids in vivo, which comprises administering the nucleic acid, or a vector (e.g., AAV vector) comprising the nucleic acid. In some embodiments, the administration is intratumoral.

In other embodiments, the cells are administered nucleic acids encoding a fusion protein that comprises a RIPK1 effector domain with an amino acid sequence as set forth in SEQ ID NO:28, or a functional variant thereof comprising an amino acid sequence with at least about 80%, about 85%, about 90%, about 95%, about 98%, or about 99% sequence identity thereto. In one embodiment, the fusion protein comprises an amino acid sequence as set forth in SEQ ID NO:30, or a functional variant thereof comprising an amino acid sequence with at least about 80%, about 85%, about 90%, about 95%, about 98%, or about 99% sequence identity thereto. In some embodiments, the multimerization domain in the fusion protein has a sequence set forth in one of SEQ ID NOS:40-44, 58-135, and 147-149. In some embodiments, the multimerization domain in the fusion protein is a trimerizing domain or higher level multimerization domain. In some embodiments, the fusion protein comprises the trimerizing domain SEQ ID NO:43. In some embodiments, the fusion protein comprises the trimerizing domain SEQ ID NO:147. In other embodiments, the fusion protein comprises the heptamerizing domain SEQ ID NO:44. In one embodiment, the cell is administered the nucleic acids in vitro. In an alternative embodiment, the cell is administered the nucleic acids in vivo, which comprises administering the nucleic acid, or a vector (e.g., AAV vector) comprising the nucleic acid. In some embodiments, the administration is intratumoral.

In other embodiments, the cells are administered nucleic acids encoding a fusion protein that comprises a RIPK2 effector domain with an amino acid sequence as set forth in SEQ ID NO:31, or a functional variant thereof comprising an amino acid sequence with at least about 80%, about 85%, about 90%, about 95%, about 98%, or about 99% sequence identity thereto. In one embodiment, the fusion protein comprises an amino acid sequence as set forth in SEQ ID NO:33, or a functional variant thereof comprising an amino acid sequence with at least about 80%, about 85%, about 90%, about 95%, about 98%, or about 99% sequence identity thereto. In some embodiments, the multimerization domain in the fusion protein has a sequence set forth in one of SEQ ID NOS:40-44, 58-135, and 147-149. In some embodiments, the multimerization domain in the fusion protein is a trimerizing domain or higher level multimerization domain. In some embodiments, the fusion protein comprises the trimerizing domain SEQ ID NO:43. In some embodiments, the fusion protein comprises the trimerizing domain SEQ ID NO:147. In other embodiments, the fusion protein comprises the heptamerizing domain SEQ ID NO:44. In one embodiment, the cell is administered the nucleic acids in vitro. In an alternative embodiment, the cell is administered the nucleic acids in vivo, which comprises administering the nucleic acid, or a vector (e.g., AAV vector) comprising the nucleic acid. In some embodiments, the administration is intratumoral.

In yet other embodiments, the cells are administered nucleic acids encoding a fusion protein that comprises an MLKL effector domain with an amino acid sequence as set forth in SEQ ID NO:34, or a functional variant thereof comprising an amino acid sequence with at least about 80%, about 85%, about 90%, about 95%, about 98%, or about 99% sequence identity thereto. In some embodiments, the fusion protein further comprises the pseudokinase domain sequence set forth in SEQ ID NO: 35, or a functional variant thereof comprising an amino acid sequence with at least about 80%, about 85%, about 90%, about 95%, about 98%, or about 99% sequence identity thereto. In some embodiments, the multimerization domain in the fusion protein has a sequence set forth in one of SEQ ID NOS:40-44, 58-135, and 147-149. In some embodiments, the multimerization domain in the fusion protein is a trimerizing domain or higher level multimerization domain. In some embodiments, the fusion protein comprises the trimerizing domain SEQ ID NO:43. In some embodiments, the fusion protein comprises the trimerizing domain SEQ ID NO:147. In other embodiments, the fusion protein comprises the heptamerizing domain SEQ ID NO:44. In some embodiments, the fusion protein comprises an amino acid sequence as set forth in SEQ ID NO:54 or 55, or a functional variant thereof comprising an amino acid sequence with at least about 80%, about 85%, about 90%, about 95%, about 98%, or about 99% sequence identity thereto. In one embodiment, the cell is administered the nucleic acids in vitro. In an alternative embodiment, the cell is administered the nucleic acids in vivo, which comprises administering the nucleic acid, or a vector (e.g., AAV vector) comprising the nucleic acid. In some embodiments, the administration is intratumoral.

The method of this aspect can be applied to any tumor microenvironment without limitation. Exemplary solid tumors are indicated above.

Additionally, the method of this aspect can be optionally combined with other anti-cancer therapeutic strategies, which are also described in more detail above. In specific embodiments, the anti-cancer therapeutic strategies are cancer immunotherapies, such as administration of checkpoint inhibitors (e.g., PD-1 pathway inhibitor), or other immunomodulatory agents.

Formulation and Administration

The disclosure also encompasses formulations appropriate for methods of administration for application to in vivo therapeutic settings in subjects (e.g., mammalian subjects with cancer). According to skill and knowledge common in the art, the disclosed fusion proteins, encoding nucleic acids, and/or vectors comprising the nucleic acids, can be formulated with appropriate carriers and non-active binders, and the like, for administration to target specific tumor and/or cancer cells.

General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

Reference to sequence identity addresses the degree of similarity of two polymeric sequences, such as protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are provided for the purpose of illustrating, not limiting, the disclosure.

### Example 1

The following describes the initial design and implementation of exemplary embodiments of fusion proteins that are constitutively active upon expression. This work demonstrates that the fusion proteins are capable of multimerization and induction of cell death without requiring additional factors, such as ligands, to effect induction.

Introduction

The caspases, RIP kinases, and MLKL are activated by an endogenous process called “induced proximity.” Specifically, inactive precursor zymogens become active when they are induced to co-localize via interactions with other signaling proteins. This mode of activation applies to the apoptotic caspases 2, 3, 8, 9 and 10, as well as the pyroptotic caspases 1, 4, 5 and 11. These enzymes are activated endogenously by the dimerization of inactive zymogens to form fully active proteolytic dimers. The RIP kinases and MLKL, which are effectors of necroptosis, are similarly activated by induced proximity. These proteins are endogenously activated via formation of oligomers in the case of the RIPKs, and specifically formation of trimers in the case of MLKL. In all cases, proximity-induced activation occurs in large complexes in the cytosol, in response to a variety of cellular signals.

In prior investigations of these proximity-induced pathways, the inventors have created ligand-activatable forms of the caspases, RIP kinases and MLKL. In general, this approach involved fusing either full-length zymogens, or key effector domains from the zymogens, to separate domains that dimerize in response to the presence of a small-molecule ligand. The FKBPF36V domain, a modified version of the mammalian FKBP protein, which can be induced to dimerize in response to a dimeric ligand, was used for this approach. FKBP fusion allowed controlled dimerization for the activation of caspases. This domain was used to create cross-linked oligomers through the stacking of multiple FKBP domains on target proteins, allowing activation of the RIP kinases and MLKL through a similar approach. However, while informative in research settings, the FKBP/ligand approaches have been limited in their in vivo application by the high cost and limited solubility of the required ligand that is used. Thus, there is a need for methods of inducing programmed cell death, including in particular immunogenic forms of cell death.

Constitutively Active Death Inducing Fusion Proteins

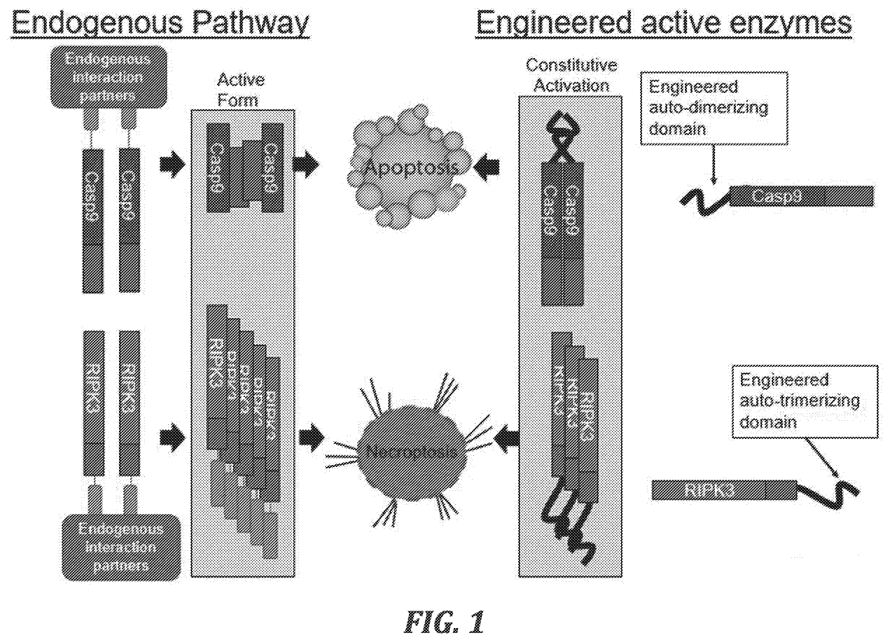

To address the issues indicated above, the inventors embarked on a wholly different approach to create constitutively active cell death effector enzymes that induce specific forms of programmed cell death upon expression or introduction into target cells. FIG. 1 provides a schematic comparing the endogenous pathway for proximity-induced induction of programmed cell death with certain embodiments of the engineered, constitutively active cell death effectors provided herein. On the left, endogenous cell death effector zymogens such as the caspases and RIP kinases are normally activated by interaction with key endogenous protein interaction partners to form active complexes. This leads to induction of cell death by apoptosis or necroptosis, respectively. In contrast, on the right, endogenous interaction domains are replaced by multimerization domains that form constitutive dimers, trimers, or even higher-order (not shown) oligomers. This leads to direct activation of cell death effectors upon their expression in cells, without requiring additional activation signals or ligands. Thus, the key death inducing domains are brought into proximity via engineered multimerization protein domains created through in silica protein design. By design the small, two helix domains are used to form specific, high affinity dimers, trimers, or higher-order oligomers (depending on the version used) in the absence of any exogenous ligand. Using this approach, constitutively active forms of the caspases, RIP kinases, MLKL or the Gasdermins were created. Expression of these constitutively active engineered enzyme constructs allowed the induction of apoptosis, pyroptosis, or necroptosis (depending on the construct used) in the absence of any exogenous ligand. These engineered cell death fusion proteins can be delivered to target cells, such as cancer cells, to induce cancer cell death and to potentiate immunotherapy approaches. Alternatively, the engineered cell death fusion proteins can introduced to cells in vitro to create cells useful for administration (e.g., to a tumor micro-environment) to induce an anti-tumor immune response.

To create constitutively active fusion proteins, wild-type interaction domains were replaced with constitutively-dimerizing, trimerizing, or higher-order oligomerizing domains. These domains were created and described in (Boyken, S. E., et al., (2016) De novo design of protein homo-oligomers with modular hydrogen-bond network-mediated specificity. Science 352, 680-687, incorporated herein by reference in its entirety). As an initial proof of concept, dimerization or trimerization domains (called 2L4HC2_23 or 5L6HC3_1, respectively) were fused to the death inducing domains of the enzymes caspase-1, caspase-8, caspase-9, caspase-11 (a murine caspase homologous to human caspase-4 and -5), RIPK1, RIPK3 and MLKL. Expression of these constructs directly and rapidly triggered apoptosis (Casp-8 or -9), pyroptosis (Casp-1, 4, 5 or 11) or necroptosis (RIPK1, RIPK3, MLKL) in target cells. The same approach was used to generate and characterize additional versions of each enzyme utilizing an alternative trimerization domain (2L6HC3_13), or hexamerization (6H8) and heptamerization (7H3) domains.

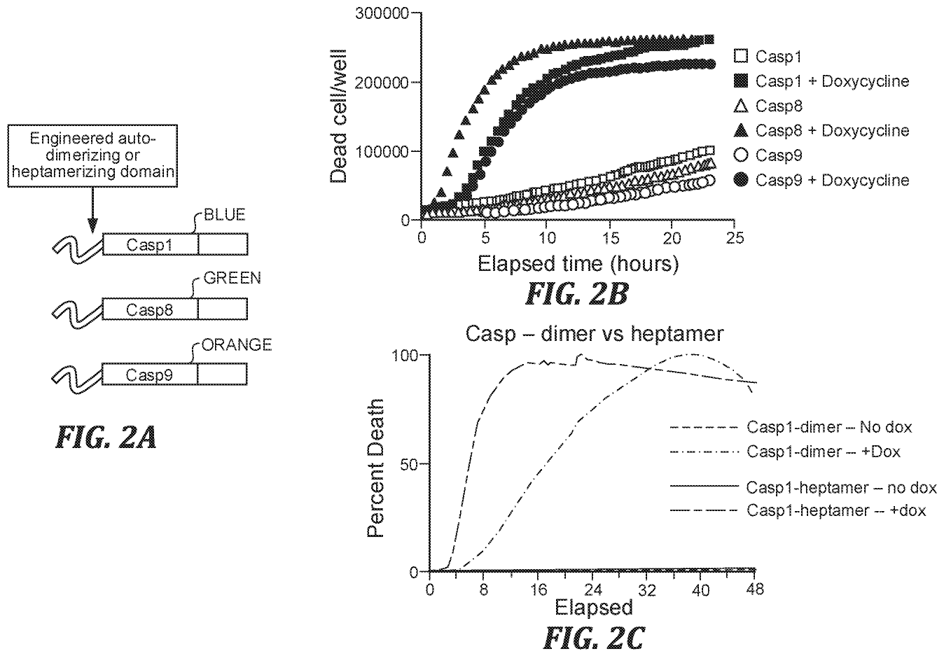

For example, FIGS. 2A-2C schematically and graphically illustrate the engineered multimerizing caspase death inducing domains and their induction of death in representative cell models. FIG. 2A is a schematic representation of the chimeric, auto-dimerizing caspase constructs. The endogenous protein interaction domain normally present at the N-terminus of the caspases was replaced by an engineered auto-dimerizing (or higher level multimerization) domain. This domain was fused to the effector domains of caspase-1 (an inducer of pyroptosis), or caspase-8 or -9 (inducers of apoptosis.) FIG. 2B graphically illustrates that the chimeric caspase constructs shown in FIG. 2A were stably transduced in murine fibroblast cells, under the control of a doxycycline-responsive promoter. In these cells, the chimeric caspase constructs were not expressed until doxycycline is added. As the cells were treated with doxycycline, cell death events were tracked over time using an IncuCyte imaging platform. Dead cells were quantified through addition of the cell-impermeable DNA-binding dye Sytox Green, which only marks cells that have lost the integrity of their plasma membrane. Expression of the constitutively-active caspase fusion proteins, in the absence of any other stimulus, was observed to be sufficient to trigger robust cell death. FIG. 2C graphically illustrates a comparison of induced expression of autodimerizing Casp-1 constructs and autoheptamerizing Casp-1 constructs. The death events were tracked as described for FIG. 2B and the expression of the constitutively-active caspase fusion proteins was sufficient to trigger robust cell death, with Casp1-heptamers demonstrating a much more immediate effect compared to dimerized Casp-1 fusion constructs. The sequences of the dimerizing fusion proteins with Casp-1 effector domains, Casp-8, and Casp-9 are set forth in SEQ ID NOS:46, 49, and 51, respectively. The sequence of the heptamerizing fusion protein with a Casp-1 effector domain is set forth in SEQ ID NO:47.

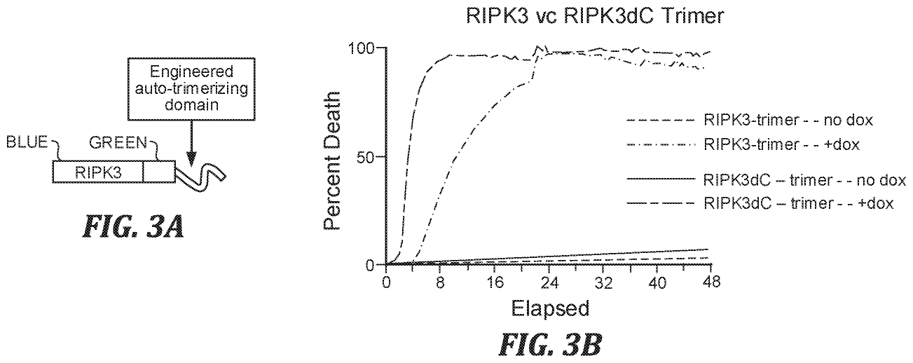

FIGS. 3A and 3B illustrate results of a similar approach to that described in FIGS. 2A-2C, using the necroptosis inducing enzyme RIPK3. As illustrated in FIG. 3A, a RIPK3 effector domain (with or without a C terminal RIP homotypic interaction motif (RHIM) as represented by the “green” domain and referred to as RIPK3dC) was fused to an engineered trimerizing domain. The sequence of the trimerizing fusion protein with a RIPK3 effector domain and RHIM domain is set forth in SEQ ID NO:52, and the sequence of the trimerizing fusion protein with an effector domain but without the RHIM domain (RIPK3dC) is set forth in SEQ ID NO:53. This construct was expressed in cells under the control of doxycycline. Cell death was quantified by uptake of Sytox Green, as described above for FIGS. 2B and 2C. As above, this chimeric enzyme construct triggered robust cell death upon expression and in the absence of other stimuli or ligands.

Conclusion

These results demonstrate the successful creation of fusion proteins comprising effector domains fused to engineered multimerization domains that form constitutively active multimeric fusion proteins. The fusion proteins can be created in many different ways, allowing formation of dimers, trimers, tetramers, pentamers, and the like. For a given multimer, different protein topologies can be achieved by altering the design of the multimerization domains. These results provide proof-of-concept by creating constitutive caspase-1, 8 and 9 dimers and RIPK3 oligomers that function to induce cell programmed death, specifically apoptosis and necroptosis, respectively. Similar processes can be employed to create constitutively active forms of any caspase, RIP kinase, or MLKL (or fragments thereof including functional death inducing domains). Thus, constitutively active enzymes can be created that are capable of triggering cell death and its associated signaling modalities.

### Example 2

The following describes additional embodiments of the disclosure, specifically addressing induction and/or introduction of a specific form of programmed cell death (PCD), necroptosis, in the microtumor environment.

Significance

The specific induction of immunogenic forms of PCD within the tumor microenvironment has been a challenge because tumor cells often mutate or lose signaling pathway components related to cell death. This study uses reductionist systems to define necroptosis as a cell death program within the tumor microenvironment that promotes beneficial anti-tumor immunity. It also demonstrates the development and optimization of engineered AAVs to trigger necroptosis in tumor cells, offering novel approaches for treating tumors by driving tumor cell death. This study further shows that these approaches synergize with established immunotherapies, including checkpoint inhibitors. Considering that successful tumor immunotherapy regimens will likely require the rational application of multiple treatment strategies, maximizing the immunogenicity of dying cells within the tumor microenvironment through specific induction of necroptosis is demonstrated to be a beneficial treatment approach for clinical applications.

Introduction

Tumor immunotherapy, which boosts the ability of the body's own immune system to recognize and kill transformed cells, constitutes an immensely successful advance in the modern treatment of cancer. Notably, the efficacy of existing T cell-targeted therapies such as immune checkpoint blockade (ICB) can often be boosted upon co-administration of cytotoxic treatments such as irradiation. However, the specific forms of PCD initiated upon administration of cytotoxic therapies to tumor cells are often not rigorously defined. Considering the growing body of evidence supporting differential immune activation or suppression in response to distinct PCD modalities, strategies to maximize the immunogenicity of dying tumor cells could potentially function to boost the effects of co-administered treatments including ICB.

Necroptosis is a form of PCD that occurs downstream of the receptor-interacting protein kinases RIPK1 and RIPK3, which assemble into an oligomeric complex termed the ‘necrosome’. A growing body of evidence supports the idea that necroptosis is a more potently immunogenic form of PCD than apoptosis in certain contexts. Necroptotic cells undergo rapid membrane permeabilization, leading to the release of intracellular contents including immunogenic DAMPs that can activate innate immune pattern recognition receptors. Furthermore, death-independent functions of RIPK3 have also been recently defined, including inflammatory chemokine and cytokine production that can promote cross-priming of CD8⁺ T cell vaccination responses and confer protection during viral infection. Therefore, a model emerges in which necroptosis can function as an alternative PCD modality that can eliminate caspase-compromised cells in the event of infection, while simultaneously releasing a payload of inflammatory signals to recruit and activate immune cells. Notably, these findings have not yet been comprehensively applied to the field of tumor immunology, in part due to technical limitations related to the manipulation of PCD programs in vivo. Indeed, specific modulation of necroptosis using endogenous signaling components is difficult, as there is extensive regulatory cross-talk between extrinsic apoptotic and necroptotic signaling pathways. This is further complicated by the fact that many tumors have mutated or silenced either caspases or the RIP kinases. Given these obstacles, the specific differential effects of enforced RIPK3 activation versus caspase-8 or -9 activation within the tumor microenvironment have not been described until the present study.

This disclosure describes a beneficial role for RIPK3-dependent necroptosis within the tumor microenvironment. Using engineered versions of pro-death enzymes, we present a reductionist system that circumvents endogenous pro-death signaling pathways within tumor cells. Importantly, ectopic activation of RIPK3 promotes tumor antigen loading by tuAPCs associated with enhanced CD8⁺ leukocyte-mediated anti-tumor responses, which leads to systemic tumor control that synergizes robustly with ICB co-administration. These beneficial effects occur specifically following administration of necroptotic cells within solid tumors, but not following exposure to apoptotic cells or cells dying via lytic necrosis (a dying process that is not a type of programmed cell death), indicating that these protective effects are due to signals specifically derived from the RIPK1/RIPK3 necrosome complex. Additionally, a tractable system for the induction of necroptosis in tumor cells in situ using novel AAVs is described, which successfully recapitulate tumor control effects following necroptosis initiation. Collectively, these findings demonstrate that engagement of necroptosis in established solid tumors promotes robust anti-tumor immunity.

Results

Necroptotic Cells Confer Tumor Control Across Multiple Syngeneic Flank Tumor Models

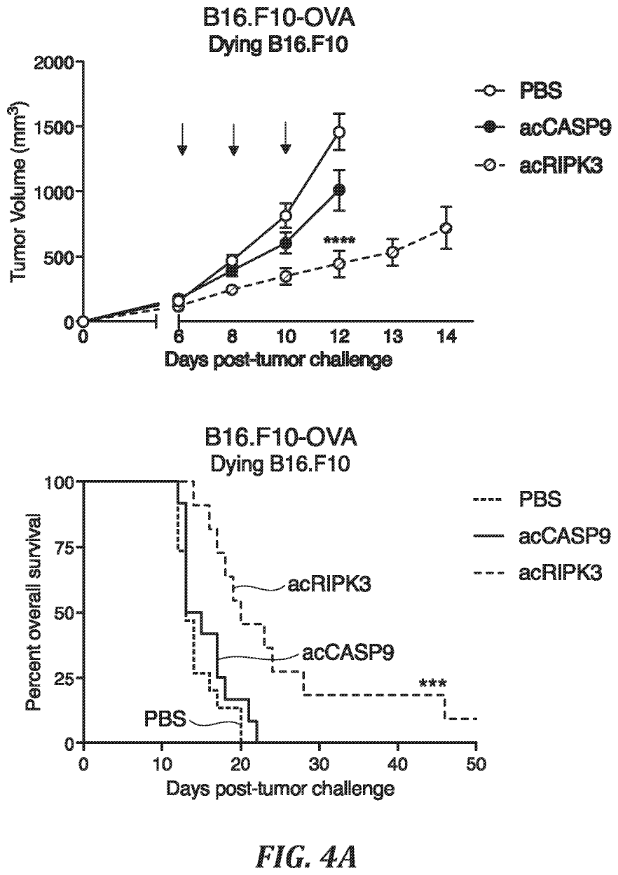

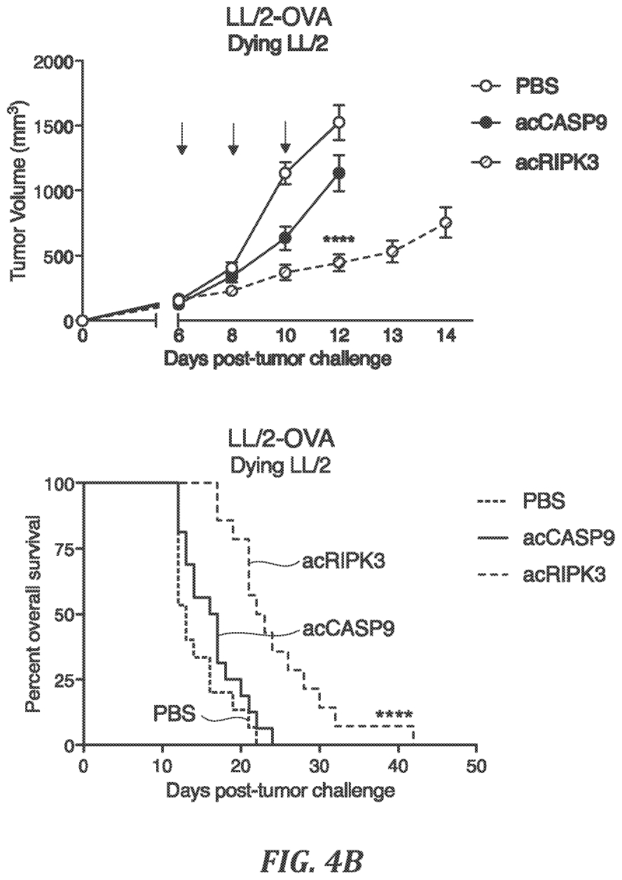

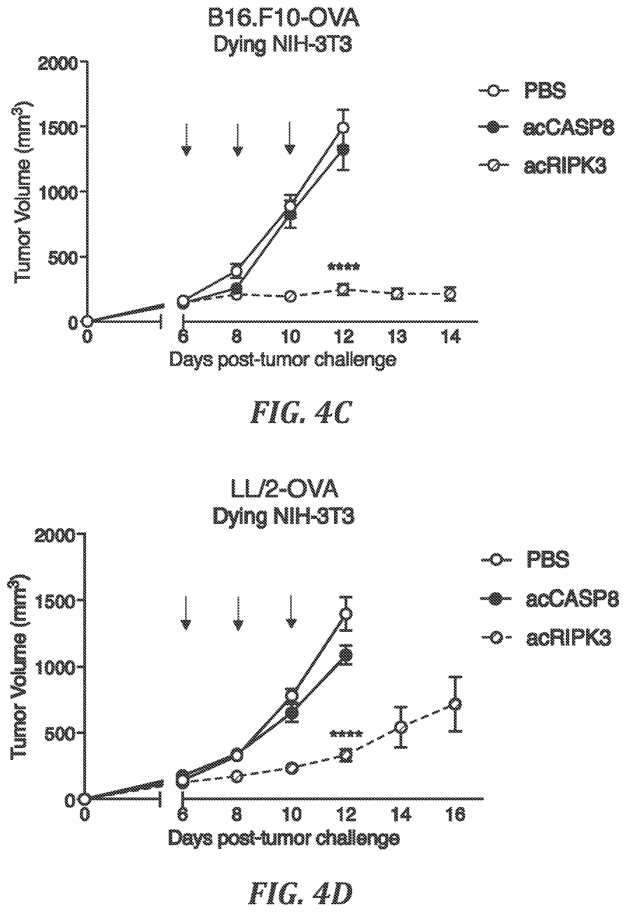

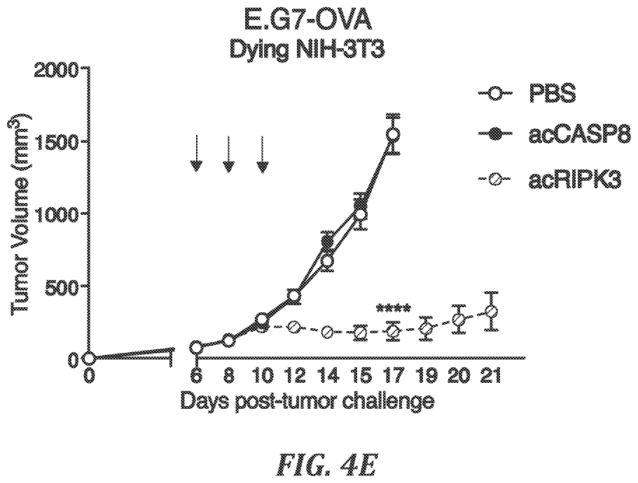

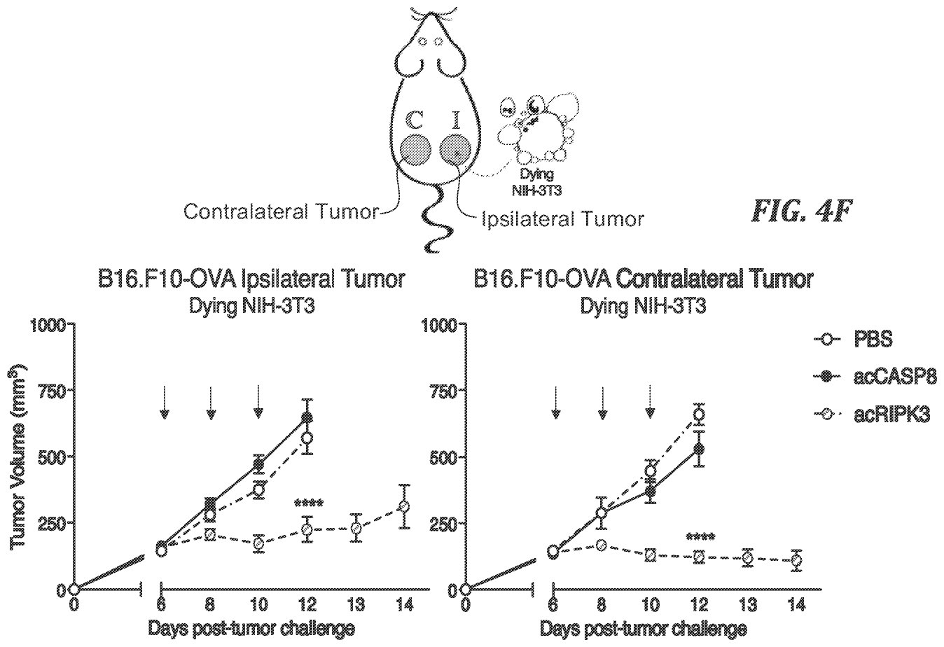

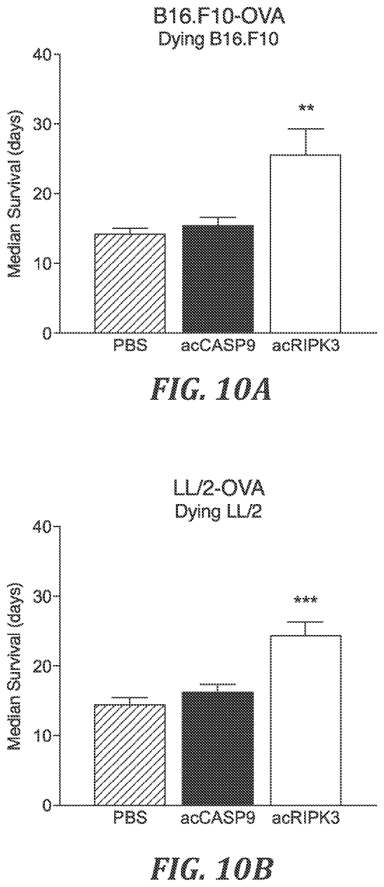

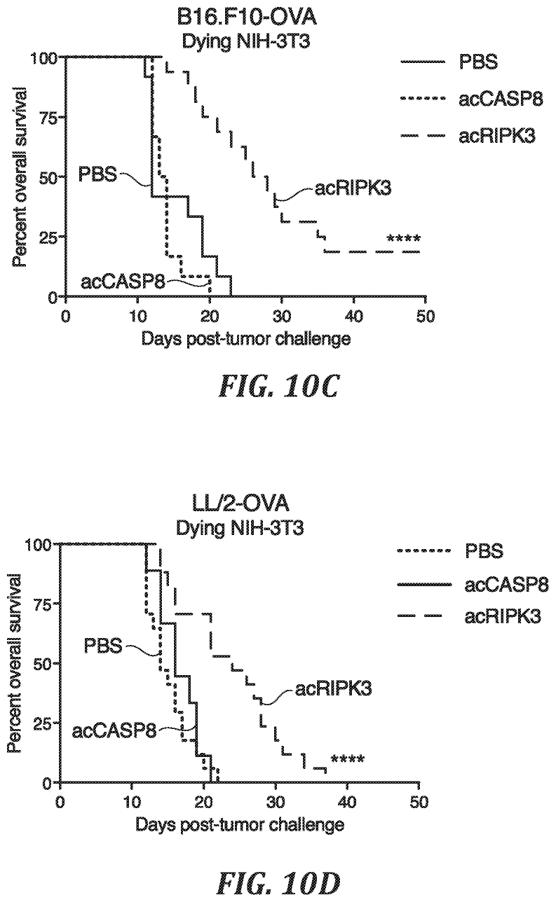

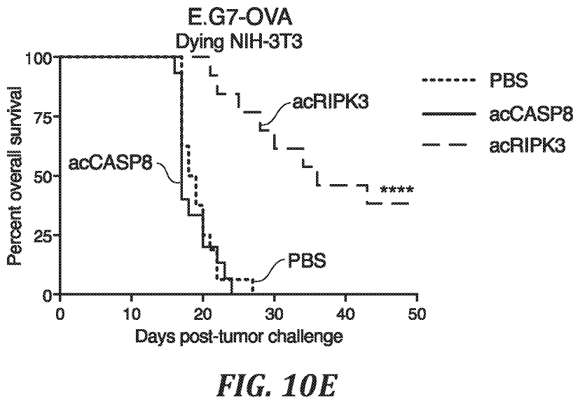

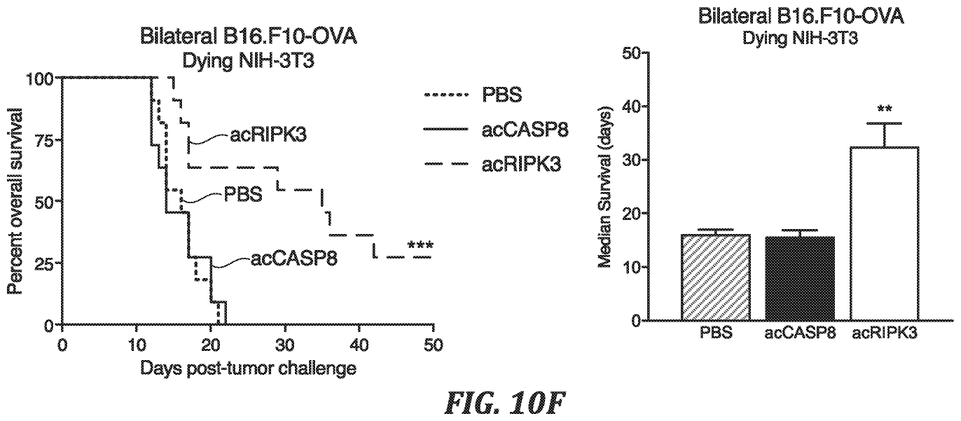

To assess the impact of necroptotic tumor cell death on gross tumor outgrowth responses, a model of intratumoral dying cell administration was utilized. Constructs encoding chimeric versions of pro-death proteins fused to activatable (“ac”) FKBP^{F36V} domains were employed, which were previously shown to allow enforced oligomerization of the chimeric protein following incubation with a synthetic bivalent homologue of rapamycin that functions as a nontoxic ligand (Orozco S., et al., (2014). RIPK1 both positively and negatively regulates RIPK3 oligomerization and necroptosis. Cell Death Differ. 21, 1511-1521, incorporated herein by reference in its entirety). Tumor cells transduced with activatable versions of either pro-apoptotic caspase-9 (acCASP9) or pro-necroptotic RIPK3 (acRIPK3) were pulsed with ligand drug in vitro to enforce oligomerization of these pro-death enzymes, and then injected intratumorally into pre-established syngeneic flank tumors. In this system, ectopically administered cells are alive at the time of injection, but are fated to undergo respective forms of PCD within the tumor microenvironment (TME), accompanied by any signaling activity induced downstream of either acCASP9 or acRIPK3. Using this model, administration of autologous necroptotic, but not apoptotic, tumor cells into cell type-matched tumors were observed to confer control of tumor outgrowth, extension of animal survival, and increased median survival of animals bearing either B16.F10-OVA melanoma flank tumors (FIG. 4A, FIG. 10A) or Lewis Lung (LL/2)-OVA adenocarcinoma flank tumors (FIG. 4B, FIG. 10B). Interestingly, the tumor control effects of necroptosis were not limited to autologous tumor cells that were fully matched for endogenous tumor-associated antigens, as injection of an unrelated fibroblast line, NIH-3T3, similarly lead to tumor control and extension of animal survival following necroptotic (acRIPK3), but not apoptotic (acCASP8), fibroblast administration in B16.F10-OVA (FIG. 4C, FIG. 10C), LL/2-OVA (FIG. 4D, FIG. 10D), and E.G7-OVA thymoma (FIG. 4E, FIG. 10E) flank tumors. These data indicate that necroptotic cells delay tumor outgrowth and even confer complete tumor clearance in some animals across multiple syngeneic flank tumor models.

Considering these findings, it was next tested if this treatment could act systemically in a bilateral flank tumor model, where a single mouse is implanted with separate B16.F10-OVA tumors on either flank (FIG. 4F). Upon injection of necroptotic fibroblasts into the tumor on one flank, control of flank tumor outgrowth was observed in both the treated (ipsilateral) as well as the untreated (contralateral) tumor (FIG. 4F), leading to an extension of animal survival and increase in median survival time (FIG. 10F). Control of both tumors in this bilateral tumor model is indicative of an abscopal effect, whereby application of a therapeutic agent to a primary tumor can lead to the control and even elimination of distal, untreated metastases. These data indicate that, once ectopically introduced into the tumor microenvironment, stimuli derived from necroptotic cells confer control of both treated and distal tumors, irrespective of antigen matching between necroptotic cells and the tumor cells themselves.

Tumor Control by Non-Autologous Necroptotic Cells Requires RIPK1/RIPK3 Activation

Given this observation that RIPK3-dependent necroptosis enabled tumor control even in the absence of tumor-associated antigens within the dying cells, the mechanisms by which necroptotic NIH-3T3 cells promote tumor control were next explored. The inventors posited that due to its lytic nature, cells undergoing necroptosis could function as a method for delivering a bolus of immunogenic DAMPs to the TME. To test this, fibroblasts dying via lytic necrosis were administered into established B16.F10-OVA tumors, using 3 different forms of lytic necrotic fibroblasts: (1) cells expressing a mutated version of activatable RIPK3 lacking the C-terminal RIP Homotypic Interaction Motif (RHIM) domain (acRIPK3ΔC), which cannot recruit and activate RIPK1 to induce downstream NF-kB-mediated inflammatory gene transcription, yet maintains the ability to activate the executioner protein mixed-linkage kinase-like (MLKL) to induce pore formation and lytic cell death, (2) cells expressing an activatable version of MLKL (acMLKL) to induce pore formation and lytic cell death in the absence of upstream RIPK3 activation, and (3) cells that were mechanically lysed via repeated freeze/thaw cycles in vitro immediately prior to intratumoral injection in vivo. All 3 forms of lytic necrotic cells similarly release cell-associated DAMPs due to loss of plasma membrane integrity, but lack activation of the RIPK1/NF-κB signaling axis that is otherwise observed upon activation of full-length RIPK3 in necroptotic NIH-3T3s.

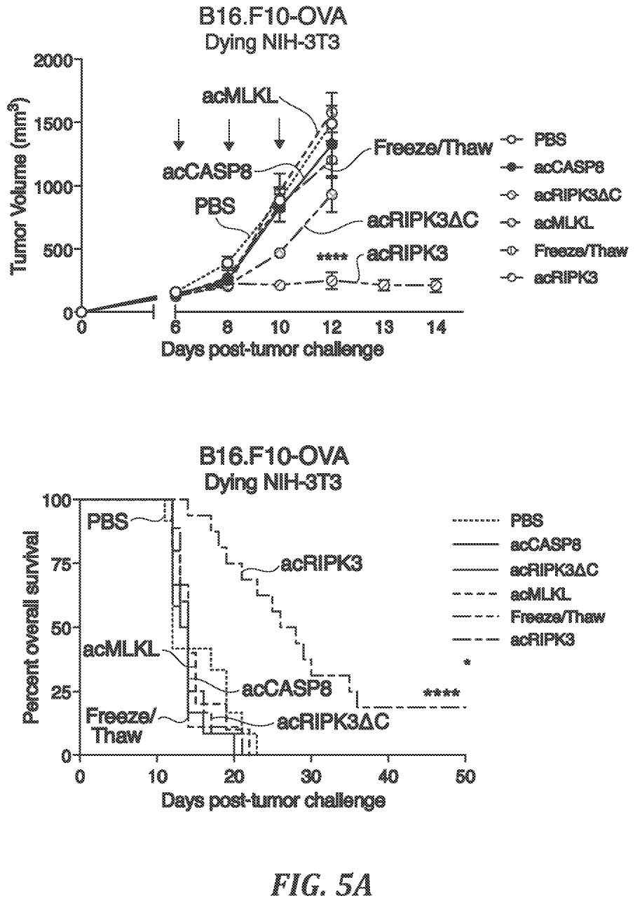

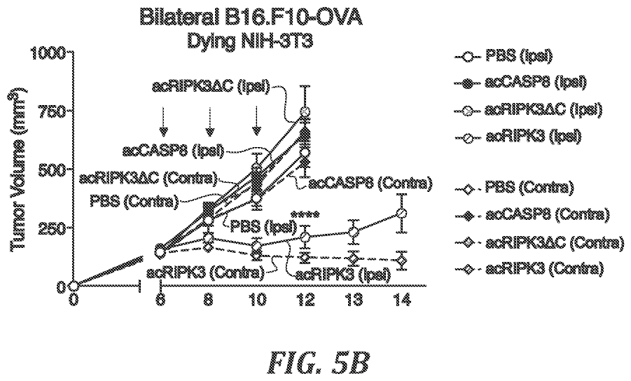

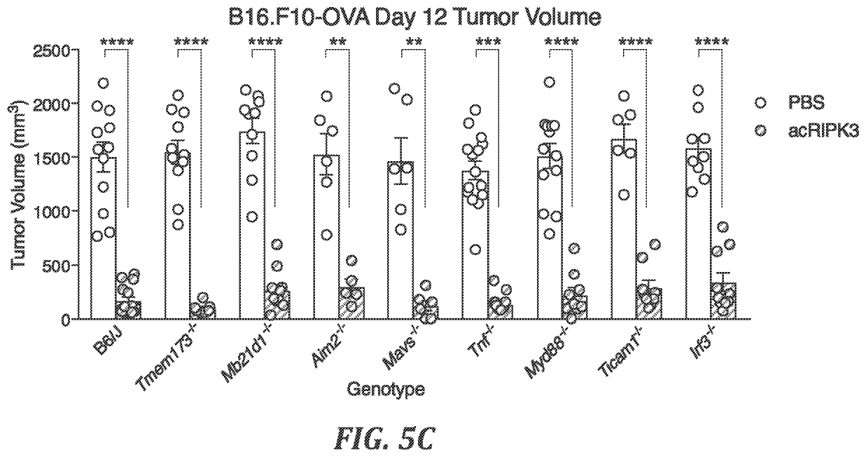

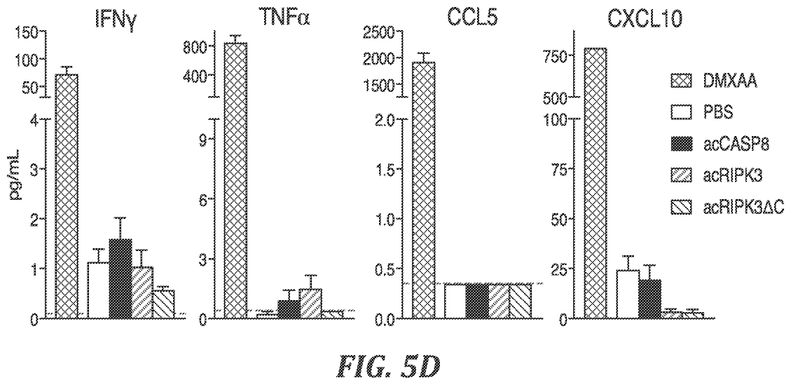

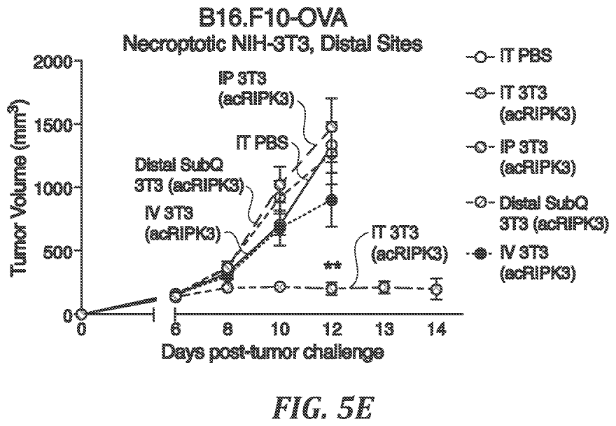

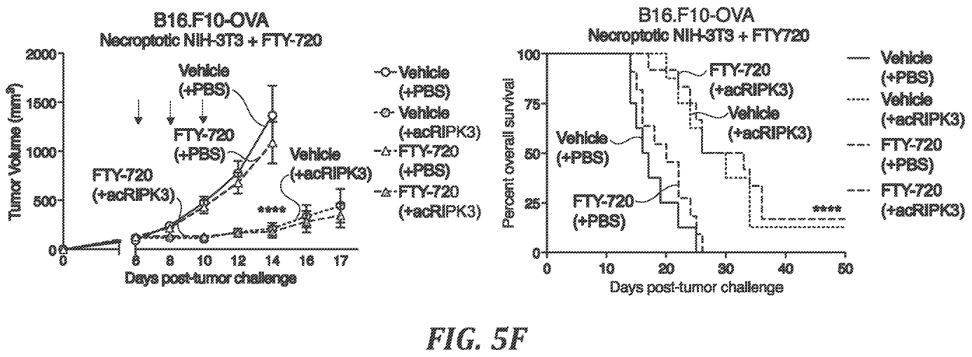

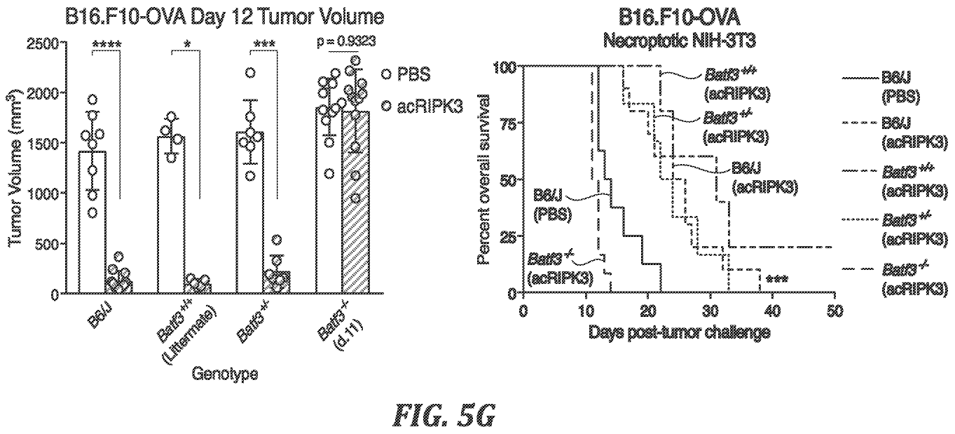

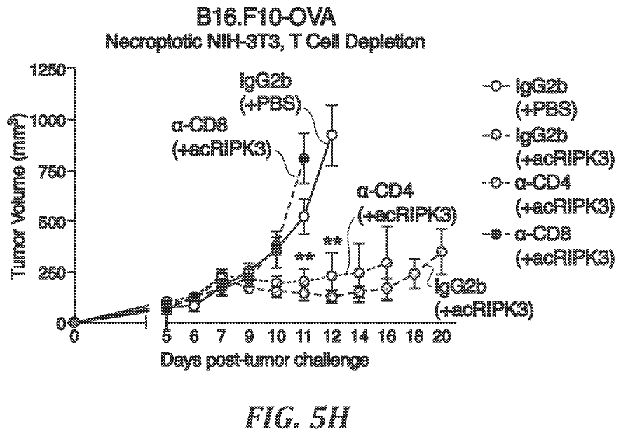

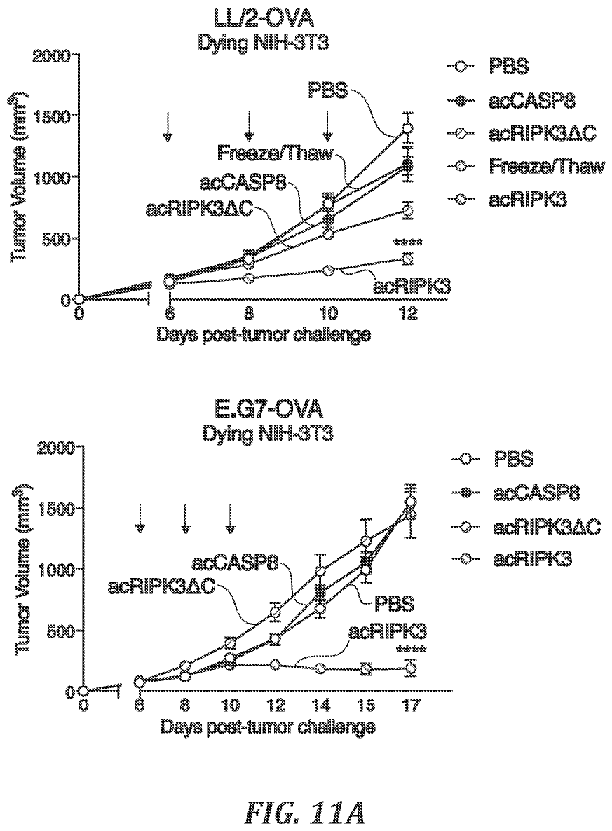

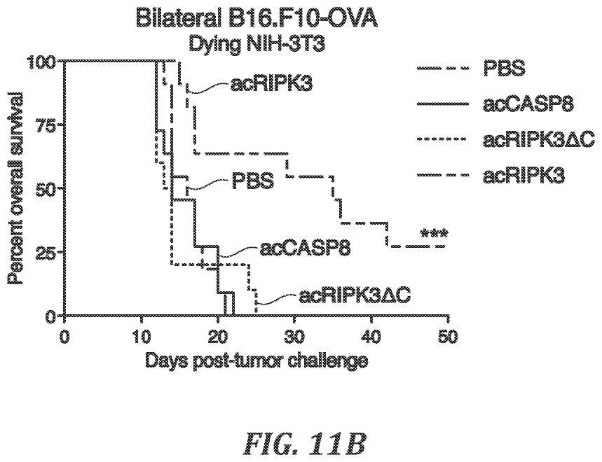

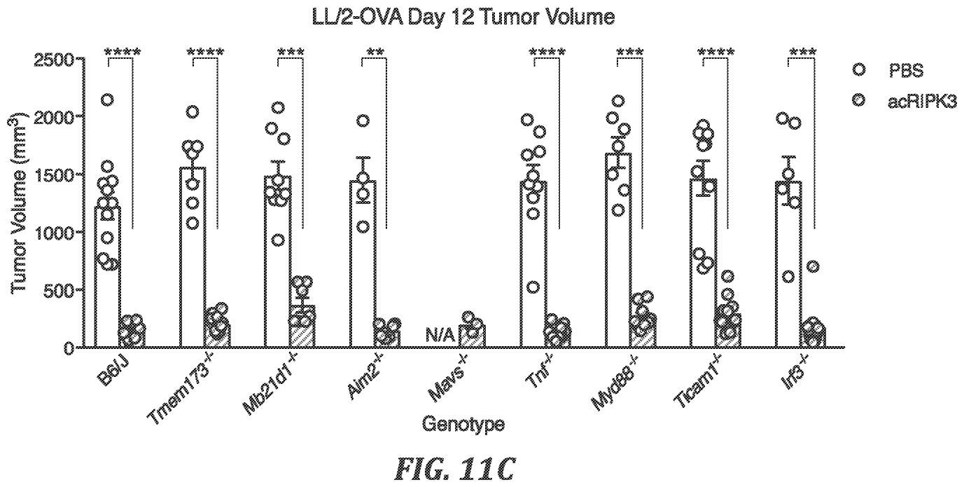

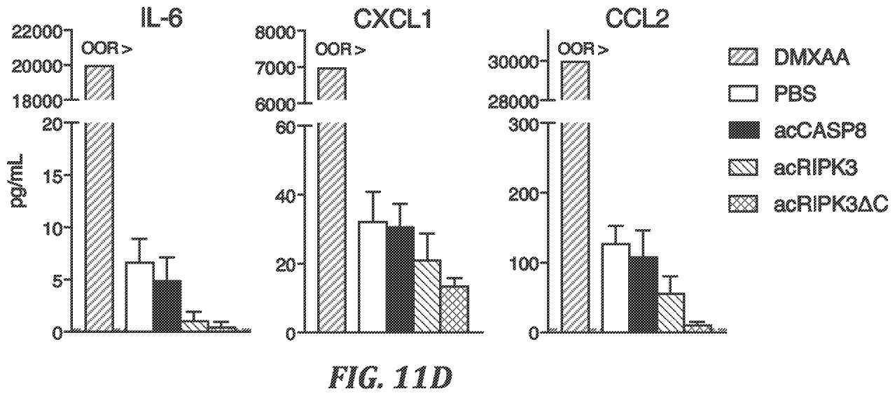

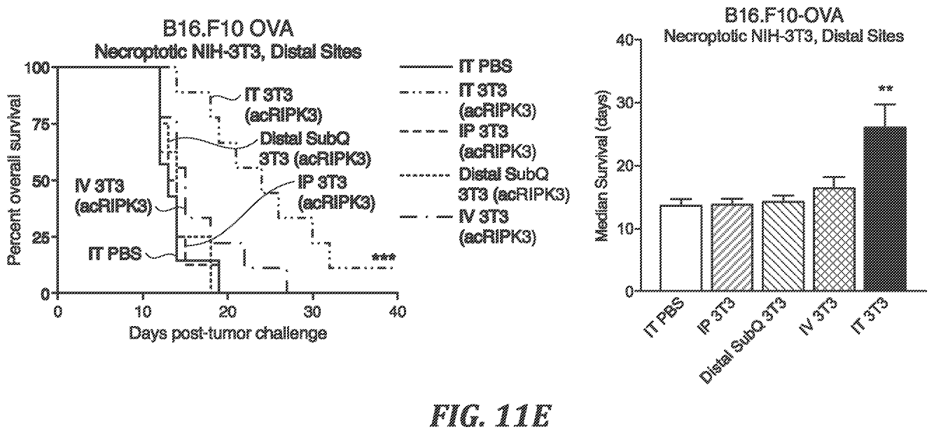

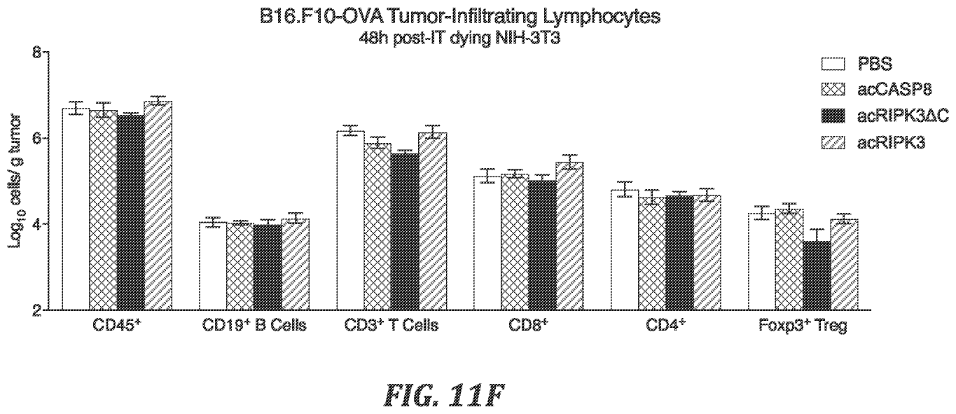

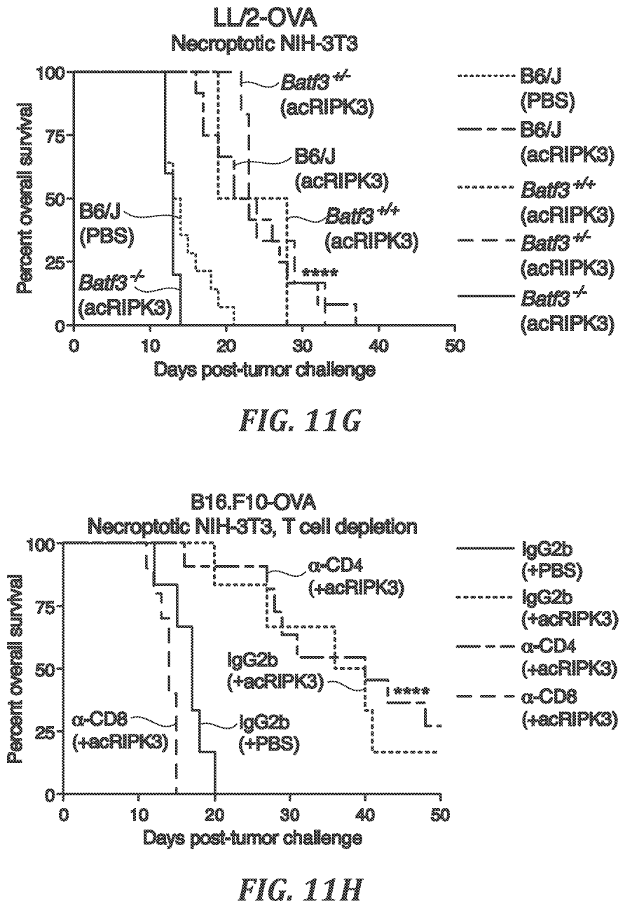

It was observed that all three treatments of lytic necrotic fibroblasts failed to confer tumor control and extend animal survival compared to fibroblasts dying via acRIPK3-mediated necroptosis, both in single B16.F10-OVA tumors (FIG. 5A) and single LL/2-OVA tumors (FIG. 11A). Consistent with this, administration of acRIPKΔC fibroblasts failed to confer tumor control (FIG. 5B) and extension of survival (FIG. 11B) in bilateral B16.F10-OVA tumor-bearing mice. These results revealed that DAMP release and subsequent pattern recognition receptor (PRR) activation are not responsible for driving the tumor control effects of necroptotic fibroblasts within the TME. To further confirm this finding, necroptotic fibroblasts was tested for the ability to recapitulate tumor control effects in a variety of global knockout mice, whose tumor-infiltrating immune cells lack expression of PRRs or their downstream signaling components. Indeed, mice deficient in signaling components involved in cytosolic DNA sensing (Tmem173^{-/-}, Mb21d1^{-/-}, Aim2^{-/-}), cytosolic RNA sensing (Mavs^{-/-}), TLR signaling (Myd88^{-/-}, Ticam1^{-/-}, Irf3^{-/-}) or general inflammation (Tnf^{-/-}) all retained the ability to control tumor outgrowth following administration of necroptotic fibroblasts into either B16.F10-OVA (FIG. 5C) or LL/2-OVA (FIG. 11C) tumors, indicating that the therapeutic effects of necroptotic cells are not strictly mediated through the singular effect of any of these innate immune signaling components within tumor-infiltrating leukocytes. Taken together, these results indicate that tumor control by necroptotic cells occurs independently of DAMP release and PRR recognition, and instead requires signaling activities downstream of RIPK1/RIPK3 necrosome complex formation and activation.

Necroptotic Cells Act Locally within the TME to Promote Immune-Mediated Tumor Control

Necroptotic NIH-3T3 cells produce a variety of inflammatory chemokines and cytokines, including CXCL1, CCL2, and IL-6, and the generation of these immunogenic signals depends on RIPK1-mediated NF-κB activation. Based on this knowledge, intratumoral injection of necroptotic fibroblasts was tested for whether they initiated systemic inflammation in recipient animals by measuring levels of inflammatory mediators in sera harvested from B16.F10-OVA tumor-bearing mice 48 hours following intratumoral necroptotic cell administration. Notably, there were no differences between treatment groups with respect to systemic levels of inflammatory chemokines and cytokines relevant for anti-tumor responses, including IFN-γ, TNF-α, CCL5, and CXCL10 (FIG. 5D), or chemokines and cytokines known to be produced by necroptotic NIH-3T3 fibroblasts, including IL-6, CXCL1, and CCL2 (FIG. 11D). Consistent with this, injection of necroptotic fibroblasts into spatially distinct locations distal from the tumor site, including intraperitoneally, intravenously, or subcutaneously on the opposite flank to the tumor, all failed to confer tumor outgrowth control (FIG. 5E) or extend animal survival (FIG. 11E) compared to intratumoral injection of necroptotic fibroblasts. These data indicate that administration of necroptotic fibroblasts does not lead to tumor control through nonspecific systemic inflammation and that the therapeutic effect of this treatment is due to local mechanisms exerted specifically within the TME.

This localization requirement for tumor control by necroptotic cells was surprising, considering previous findings that neuronal RIPK3 activation is required for the production of CCL2 and CXCL10 to recruit CCR2⁺ monocytes and CXCR3⁺ T cells to the brain during neurotropic viral infection. This presented the possibility that activation of RIPK3 in the TME could lead to the production of chemokines important for attracting key leukocyte subsets into the tumor. To test whether necroptotic fibroblast injection required the recruitment of CD8⁺ T cells primed in the tumor-draining lymph node (tdLN) into the TME to exert tumor control, necroptotic fibroblasts were co-administered with the sphingosine-1-phosphate receptor modulator FTY-720 to inhibit egress of lymphocytes from the tdLN. Interestingly, blockade of lymphocyte trafficking did not affect B16.F10-OVA tumor control by necroptotic fibroblasts, as FTY-720-treated animals still exhibited effective control over tumor outgrowth and extension of animal survival compared to vehicle-treated controls (FIG. 5F). Indeed, enumeration of various tumor-associated lymphocyte populations isolated from B16.F10-OVA tumors 48 hours post-dying cell administration revealed similar numbers of CD19⁺ B cells, CD4⁺ T cells, and CD8⁺ T cells within the TME among tumors that received apoptotic (acCASP8), necroptotic (acRIPK3), or lytic necrotic (acRIPK3ΔC) cell injections (FIG. 11F). These results show that the therapeutic effect of necroptotic fibroblasts does not function by producing lymphocyte-recruiting chemokines into the TME, and furthermore, that rapid recruitment of tumor-reactive lymphocytes from the tumor-draining lymph node is not required for tumor control.

Given these findings, the question of which immune cell subsets were required for tumor control by necroptotic fibroblasts was investigated. Batf3 is a transcription factor required for the development of cDC1 dendritic cells, which are critical for the cross-presentation of exogenous antigens to stimulate CD8-mediated immunity, indeed, cDC1 are required for endogenous anti-tumor immune responses. The tumor control effects of necroptotic fibroblasts required Batf3⁺ cDC1, as Batf3^{-/-} mice failed to restrict B16.F10-OVA (FIG. 5G) or LL/2-OVA (FIG. 11G) tumor growth compared to wild-type B6/J, wild-type littermate (Batf3^{+/+}), or heterozygous (Batf3^{+/-}) controls. Consistent with the critical role for cDC1 in mediating anti-tumor immunity, it was also observed that depletion of CD8⁺ leukocytes (including both CD8α⁺ cytotoxic T cells and CD8α⁺ dendritic cells) completely abrogated the therapeutic effect of necroptotic fibroblast administration, while depletion of CD4⁺ leukocytes did not affect tumor control responses (FIG. 5H, FIG. 11H). Collectively, these results define a role for local induction of necroptosis within the TME that promotes Batf3⁺ cDC1- and CD8⁺ leukocyte-dependent tumor control that requires the activation of a RIPK1/RIPK3 complex.

Necroptosis Promotes Anti-Tumor CD8⁺ T Cell Responses and Synergizes with Immune Checkpoint Blockade

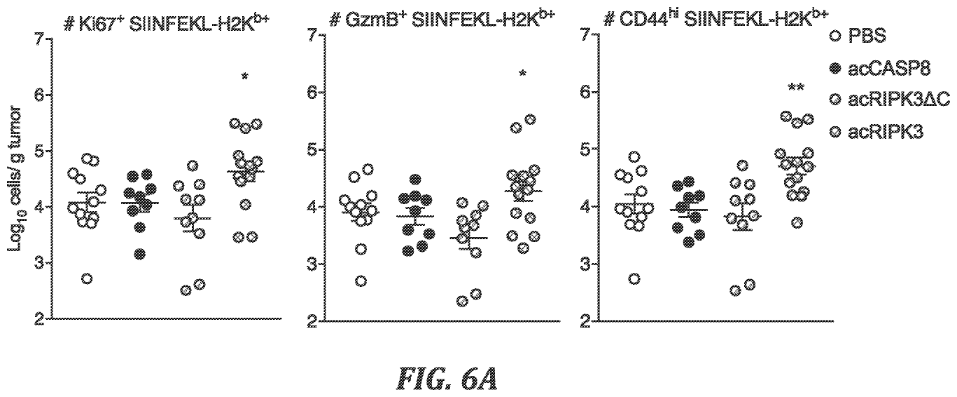

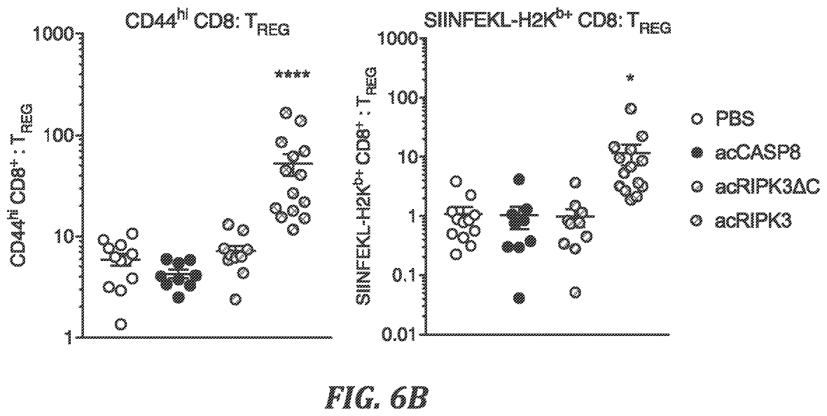

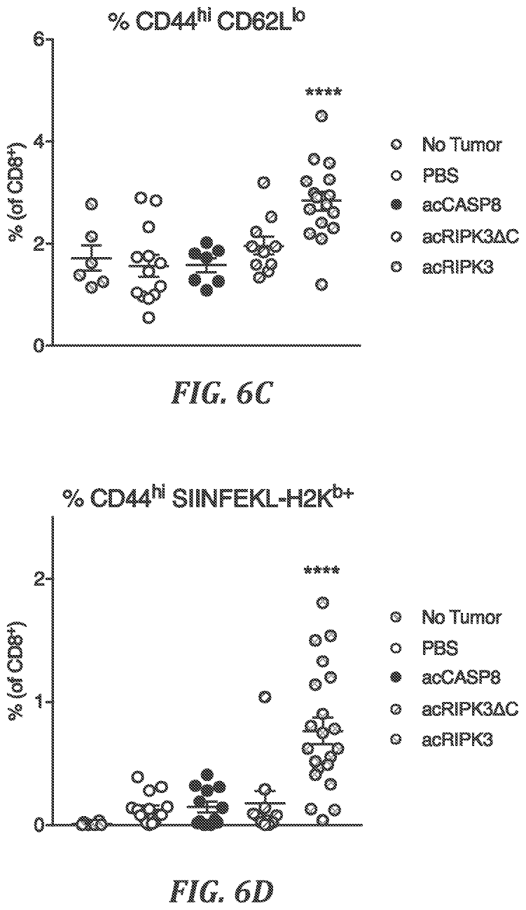

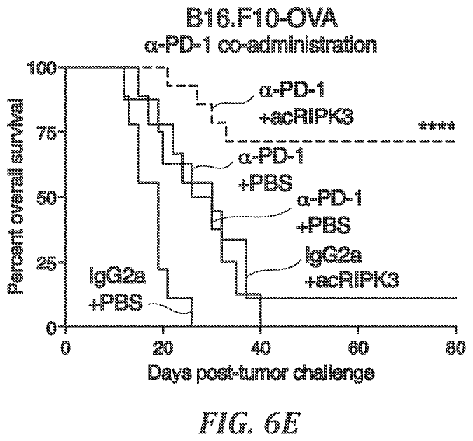

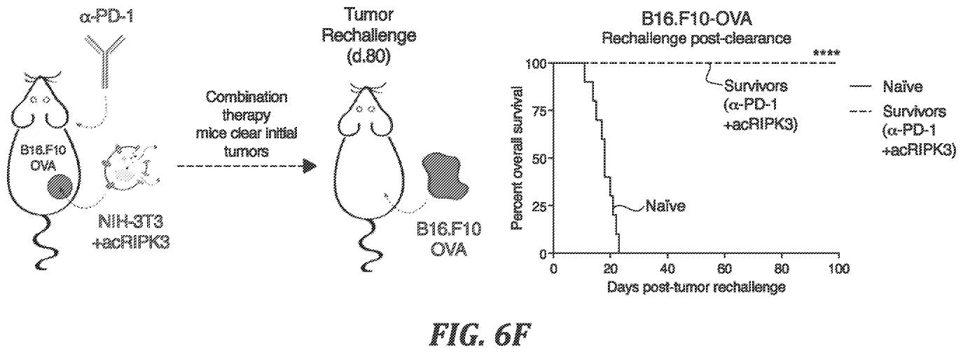

As tumor control following necroptotic fibroblast administration required a CD8⁺ cellular compartment, the next step was to characterize the effects of dying cell administration on cytotoxic CD8⁺ T cells, as they are a critical mediator of anti-tumor immunity. Using flow cytometric analysis to identify subsets of OVA-specific (SIINFEKL-H2K^{b+}) CD8⁺ T cells isolated from B16.F10-OVA tumors, increased numbers were observed of OVA-specific T cells that were positive for markers of proliferation (Ki67⁺), effector function (GranzymeB⁺), and general activation (CD44^{hi}) following necroptotic (acRIPK3), but not apoptotic (acCASP8) or lytic necrotic (acRIPK3ΔC) fibroblast administration (FIG. 6A). Furthermore, significant increases were observed in the ratios of both activated (CD44^{hi}) or tumor-specific (SIINFEKL-H2k^{b+}) CD8⁺ T cells to CD25⁺Foxp3⁺ T_{REG} (FIG. 6B) specifically within tumors that received necroptotic fibroblasts, indicating that the profile of tumor-infiltrating T cells was skewed towards more favorable cytotoxic CD8⁺ T cells, rather than an immunosuppressive profile dominated by T_{REG}. These data indicate that exposure to necroptotic cells within the TME is associated with increased numbers of tumor-specific CD8⁺ T cells present in the tumor tissue.

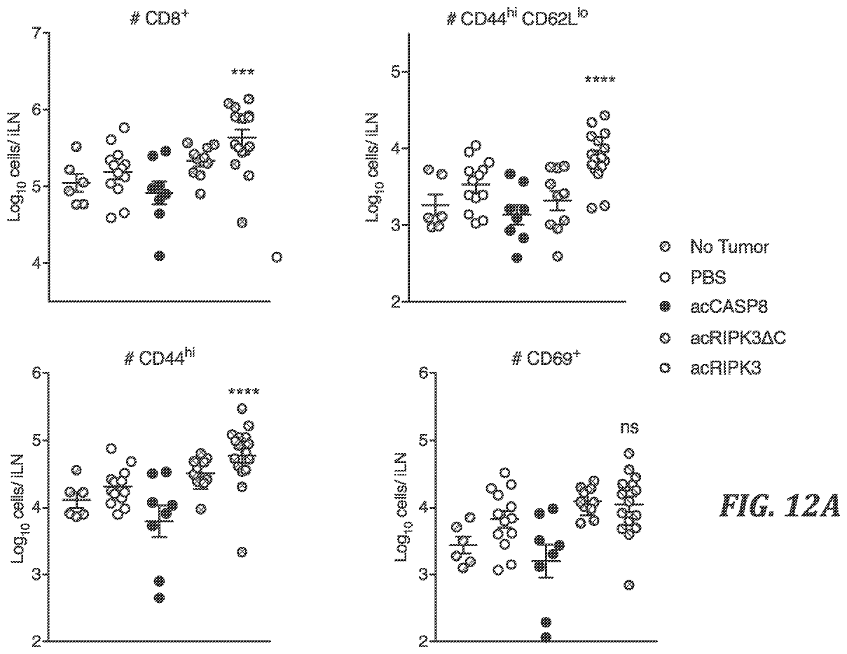

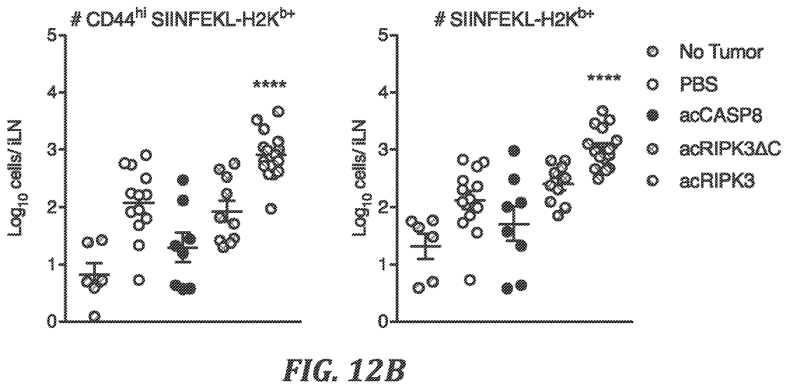

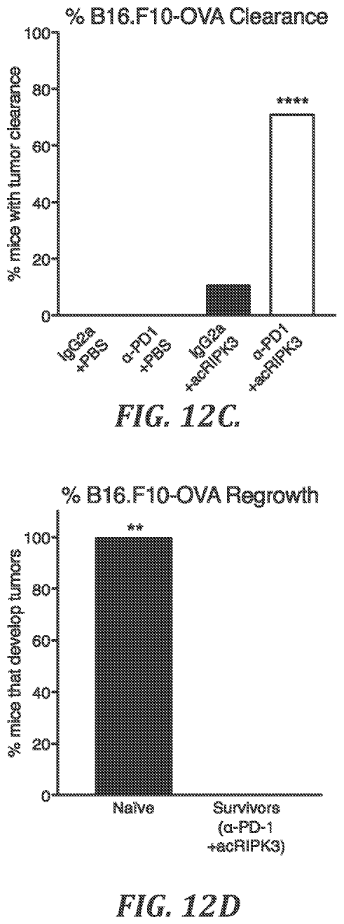

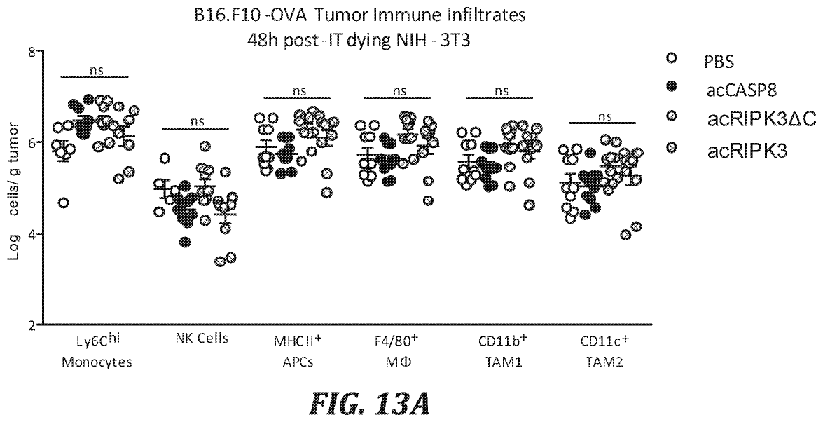

In order to characterize the effects of necroptotic cell administration on lymph node priming, the abundance and quality of CD8⁺ T cell responses in the tumor-draining lymph node (tdLN) of these mice were concurrently examined. Notably, an increased frequency (FIG. 6C) and number (FIG. 12A) of overall activated (defined as CD44^{hi} CD62L^{lo}) CD8⁺ T cells were observed in the tdLN of mice that received intratumoral necroptotic fibroblasts. These were accompanied by increases in the numbers of bulk CD8⁺ and single-positive CD44^{hi} CD8⁺ T cells, but not CD69⁺ CD8⁺ T cells (FIG. 12A). Similar increases were also observed in the frequency (FIG. 6D) and number (FIG. 12B) of activated, tumor-specific (defined as CD44^{hi} SIINFEKL-H2k^{b+}) CD8⁺ T cells in the tdLN of necroptotic cell-treated mice. Therefore, in addition to an expansion of favorable CD8⁺ T cell phenotypes locally within the TME, necroptotic fibroblast injection also resulted in lymph node priming of tumor-reactive cytotoxic CD8⁺ T cells, although priming was dispensable for single tumor control (FIG. 5F).

The efficacy of ICB is often boosted upon co-administration with cytotoxic therapies, including irradiation. Because stimuli from necroptotic cells boosted numbers of tumor-specific CD8⁺ T cells, it was hypothesized that ectopic administration of necroptotic cells could function as a stimulatory form of PCD within the TME that could potentially synergize with ICB, specifically α-PD-1. To test this, injections of necroptotic fibroblasts were interleaved into B16.F10-OVA flank tumors with administration of α-PD-1, and it was observed that 71.4% of mice successfully cleared their tumors (FIG. 12C) and exhibited significantly improved survival outcomes (FIG. 6E) following this co-administration regimen. To determine whether this successful combination therapy conferred protective immune memory, mice were re-challenged ˜2 months after they successfully cleared their tumors, injecting identical tumor cells into the same flank that bore the initial B16.F10-OVA tumor (FIG. 6F, left panel). Notably, 100% of mice were protected from tumor re-challenge (FIG. 12D) and failed to succumb to tumor outgrowth compared to naive B6/J controls (FIG. 6F, right panel). Altogether, these data indicate that necroptosis in the TME can potently synergize with ICB co-administration to promote durable tumor rejection.

Exposure to Necroptosis in the TME Promotes Antigen Uptake and Activation of Tumor-Associated APCs

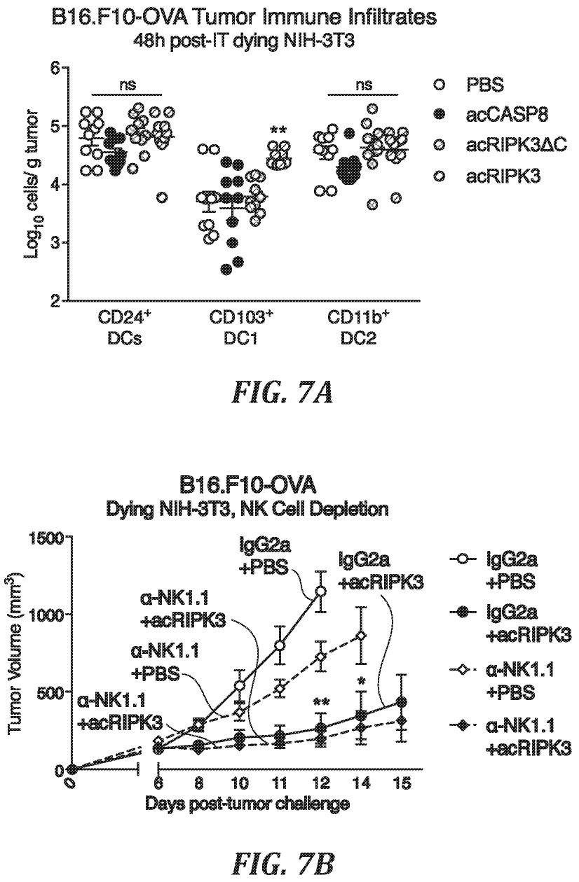

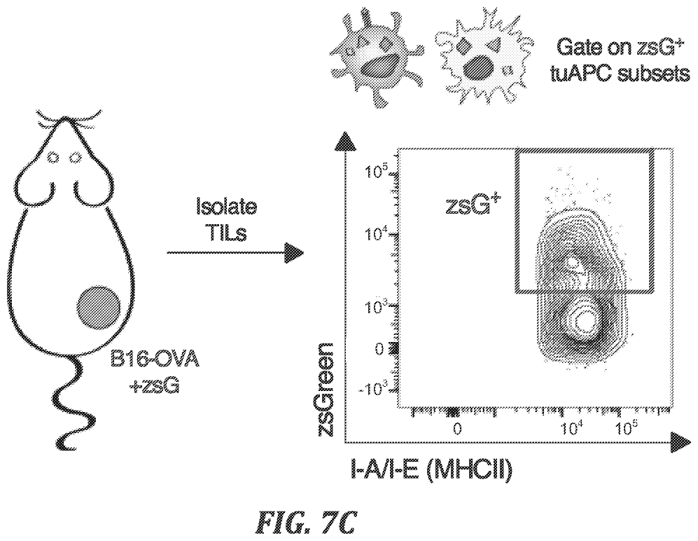

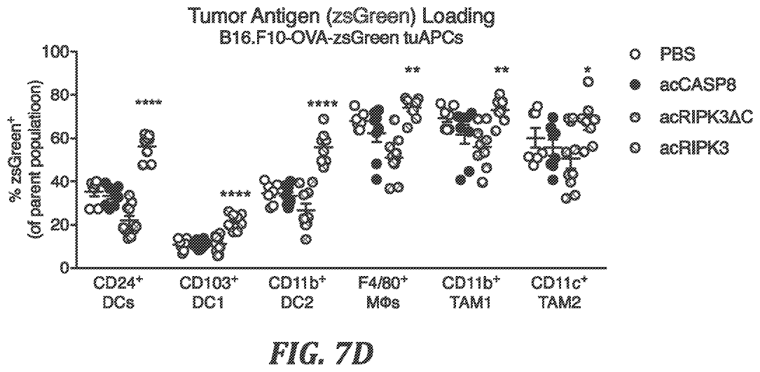

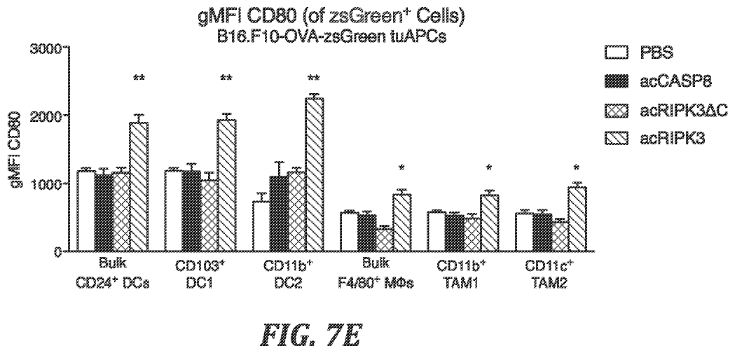

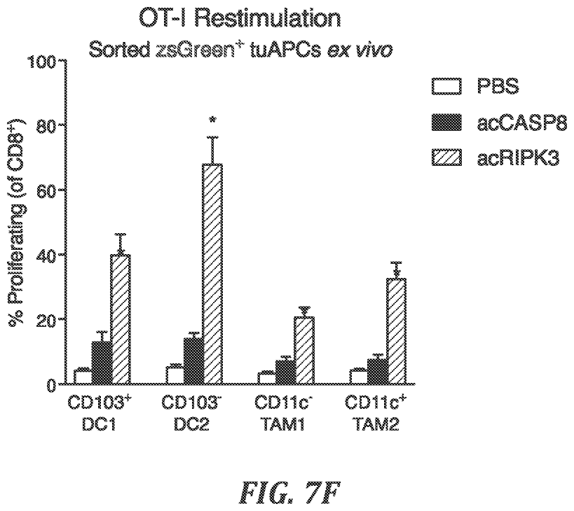

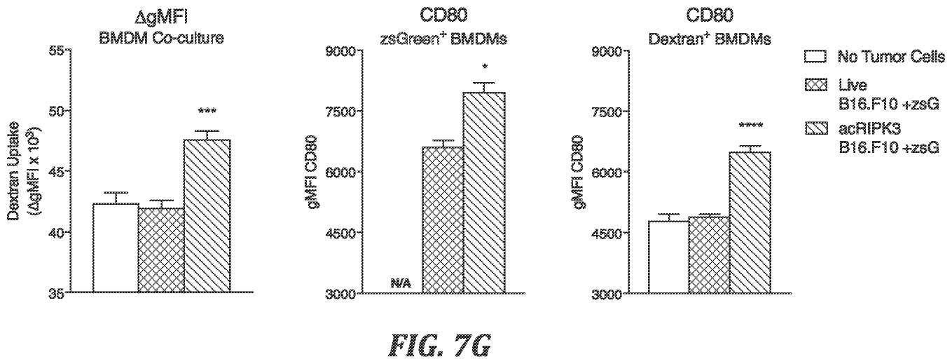

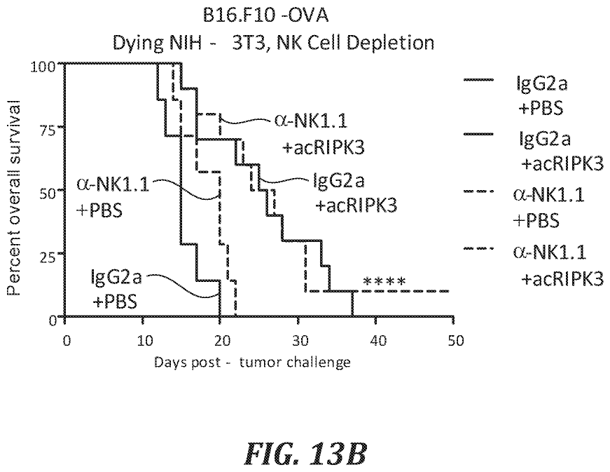

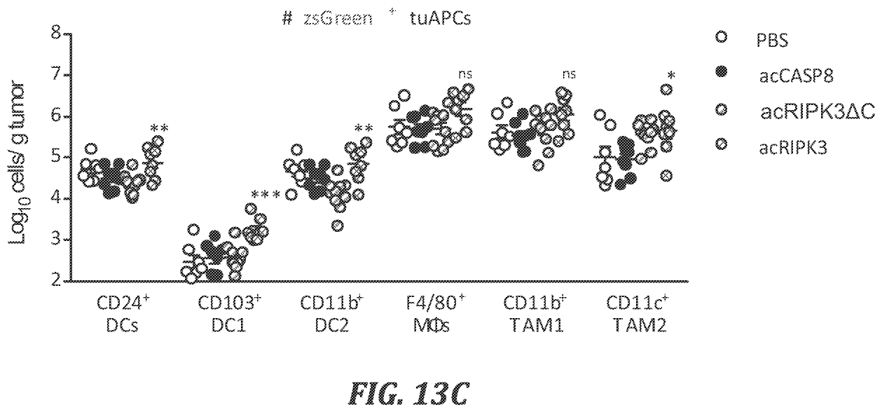

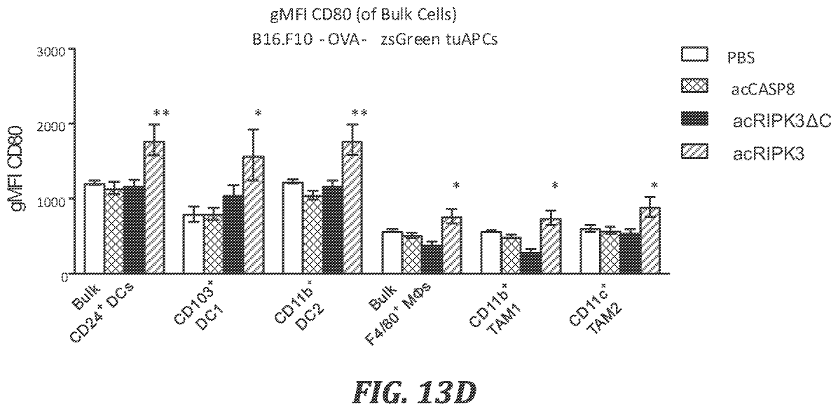

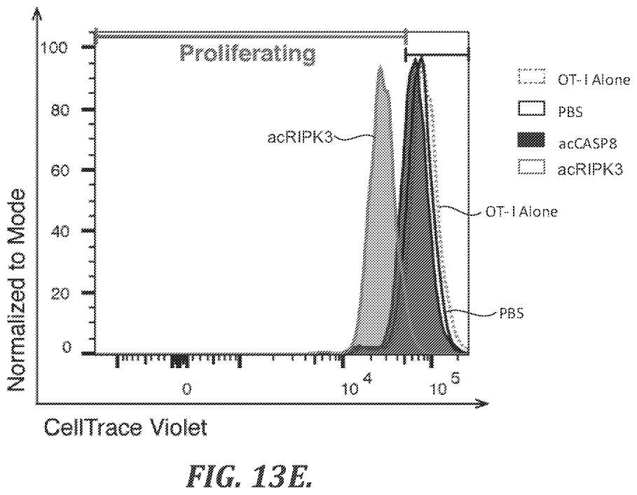

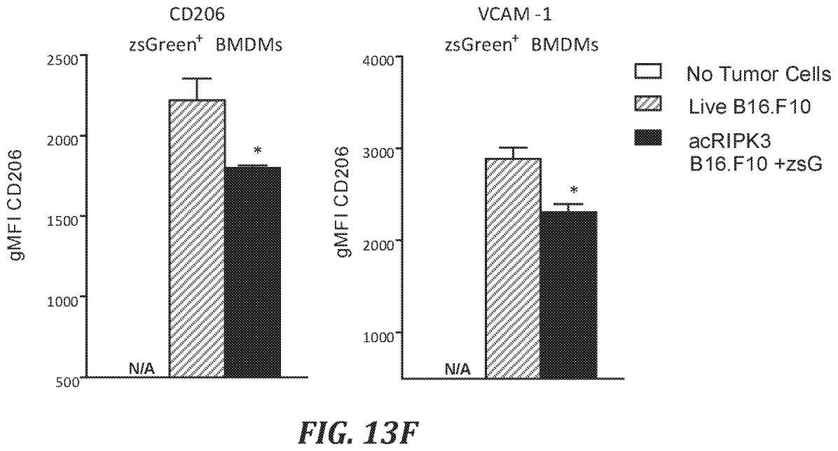

These data indicate that necroptosis potentiates anti-tumor CD8⁺ T cell responses even when necroptotic cells do not contain tumor antigen. Therefore, the next aim was to define necroptosis-induced changes to tumor-associated myeloid cell populations that could function upstream to initiate adaptive immunity. Using a previously published gating strategy to identify subsets of tumor-associated innate immune cells (Broz M. L., et al. (2014). Dissecting the tumor myeloid compartment reveals rare activating antigen-presenting cells critical for T cell immunity. Cancer Cell. 26, 638-652), various innate immune cells isolated from B16.F10-OVA tumor tissue following administration of apoptotic (acCASP8), lytic necrotic (acRIPK3ΔC), or necroptotic (acRIPK3) fibroblasts were enumerated. Notably, a significant increase was observed in the number of CD24⁺ CD103⁺ DC1 (FIG. 7A), although there were no significant differences in the number of Ly6C^{hi} monocytes, NK1.1⁺ NK cells, bulk MHCII⁺ tumor-associated antigen-presenting cells (tuAPCs), F4/80⁺ macrophages, or CD24⁺ CD11b⁺ DC2 (FIG. 7A, FIG. 13A). This was promising, given that CD103⁺ DC1 are often viewed as the most functional tuAPC subset with respect to stimulating CD8⁺ T cell-mediated anti-tumor immunity. Considering that CD103⁺ DC1 can be recruited to the TME via NK cell-derived chemokines, it was tested whether depletion of NK cells abrogated the therapeutic effect of necroptotic fibroblasts. Interestingly, NK cell depletion had no effect on tumor control (FIG. 4B) and survival extension (FIG. S4B) by necroptotic fibroblasts, indicating that either this influx of CD103⁺ DC1 into the TME was dispensable for tumor control, or that CD103⁺ DC1 were recruited to the TME via NK cell-independent mechanisms.

Beyond the overall number of tumor-associated innate immune cell populations, the phenotype of phagocytic tuAPCs with respect to tumor antigen loading and their activation status were next evaluated. To do this, mice were implanted with B16.F10-OVA cells that also express the bright and stable fluorophore zsGreen. Using zsGreen as a surrogate tumor antigen, zsGreen⁺ tumor phagocytes were gated in order to identify tuAPCs that have ingested tumor-derived material (FIG. 7C, FIG. 13C). Using this gating strategy, zsGreen⁺ subsets of 6 primary tuAPC populations were identified: bulk CD24⁺ DCs, CD103⁺ DC1, CD11b⁺ DC2, bulk F4/80⁺ tumor-associated macrophages (TAM), CD11b⁺ TAM1, and CD11c⁺ TAM2. Intriguingly, the proportion of zsGreen⁺ cells was significantly increased across all tuAPC subsets following administration of non-zsGreen-labeled necroptotic fibroblasts, this increase was particularly pronounced in the DC subsets examined (FIG. 7D). Accordingly, the absolute number of zsGreen⁺ cells among tumor DC subsets was increased following necroptotic cell exposure (FIG. 13D). As zsGreen expression was restricted to B16.F10-OVA tumor cells in this model, these results show that signals derived from necroptotic fibroblasts act in trans to increase either the rate of phagocytosis or the retention of tumor-associated antigen within tuAPC populations.

Next, the activation status of zsGreen⁺ tuAPCs was assessed following exposure to dying fibroblasts within the TME. zsGreen⁺ tuAPCs were first observed to express higher levels of the costimulatory marker CD80 on a per cell basis following administration of necroptotic fibroblasts, this increase was consistent across all 6 tuAPC subsets examined (FIG. 7E). Coupled with increased percentages of zsGreen⁺ tuAPCs following exposure to necroptotic cells, it follows that a significant increase in the gMFI of CD80 exposure to necroptotic cells was observed when gating on bulk (zsGreen⁻ and zsGreen⁺) populations of each tuAPC subset (FIG. 13E). Importantly, this increase in activation marker expression correlated with an improved functional capacity of these zsGreen⁺ tuAPCs following exposure to necroptotic cells in the TME, as zsGreen⁺ tuAPCs sorted ex vivo were capable of more robustly stimulating proliferation of previously-activated transgenic OVA-specific (OT-I) CD8⁺ T cells in an in vitro co-culture system (FIG. 7F, FIG. 13F). Therefore, exposure to necroptotic cells in the TME increases not only the abundance, but the immunostimulatory quality of tumor antigen-loaded tuAPCs.

Consistent with this, necroptotic cells were found to enhance antigen uptake by phagocytes in a tumor-independent setting, as co-culturing bone marrow-derived macrophages (BMDMs) with necroptotic B16.F10 tumor cells in vitro resulted in increased uptake of an inert dextran-fluorophore substrate included in the co-culture, compared to BMDMs cultured with live B16.F10 cells (FIG. 7G, left panel). Notably, uptake of this bystander substrate was also associated with an increase in CD80 expression on both dextran⁺ and zsGreen⁺ BMDMs only following co-culture with necroptotic B16.F10 cells (FIG. 7G, right panels), while expression of the immunomodulatory markers CD206 and VCAM-1 was decreased on zsGreen⁺ BMDMs co-cultured with necroptotic tumor cells compared to live tumor cell controls (FIG. 13G). Collectively, these data indicate that stimuli derived from necroptotic cells increase antigen loading by phagocytic cell subsets, and that this effect may constitute a conserved response to necroptotic cell-derived stimuli rather than a specific effect restricted to the TME.

Novel Adeno-Associated Viruses (AAVs) can be Used to Specifically Induce Necroptosis of Tumor Cells In Vitro

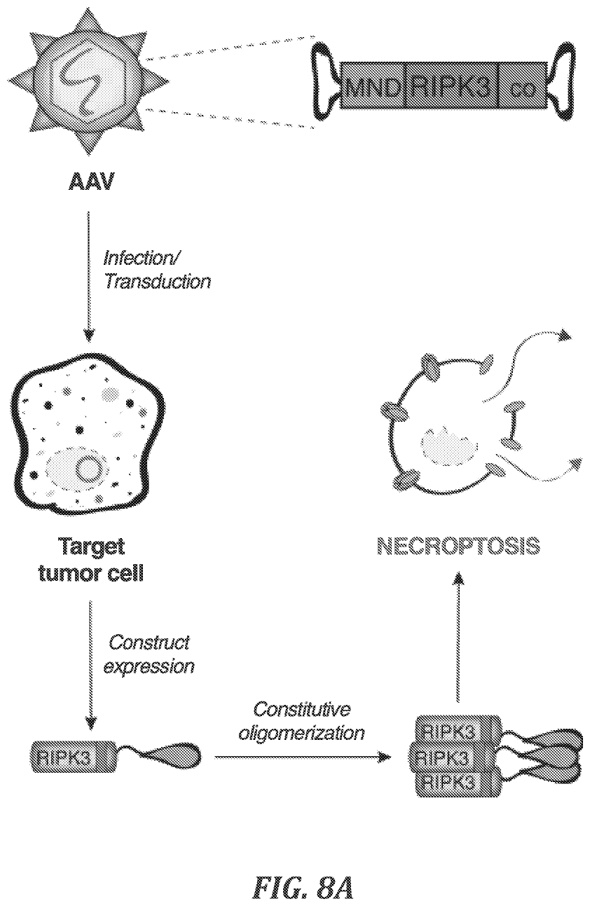

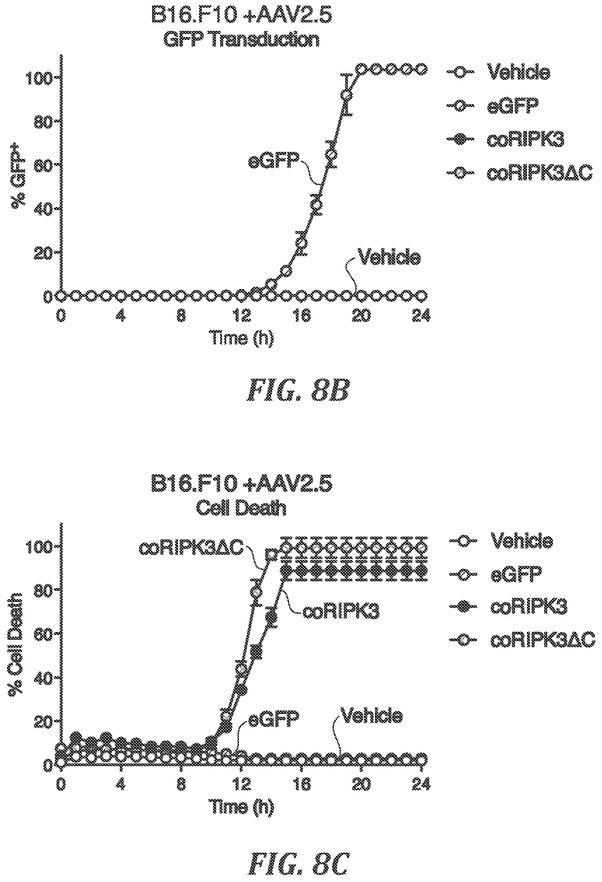

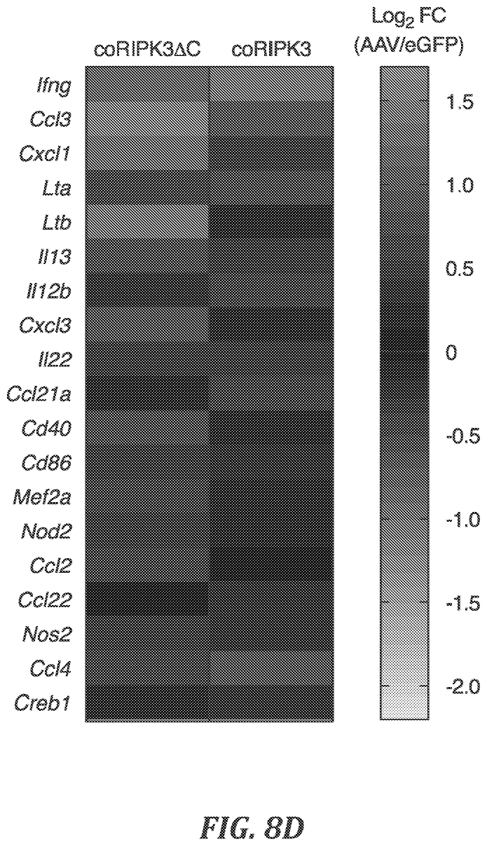

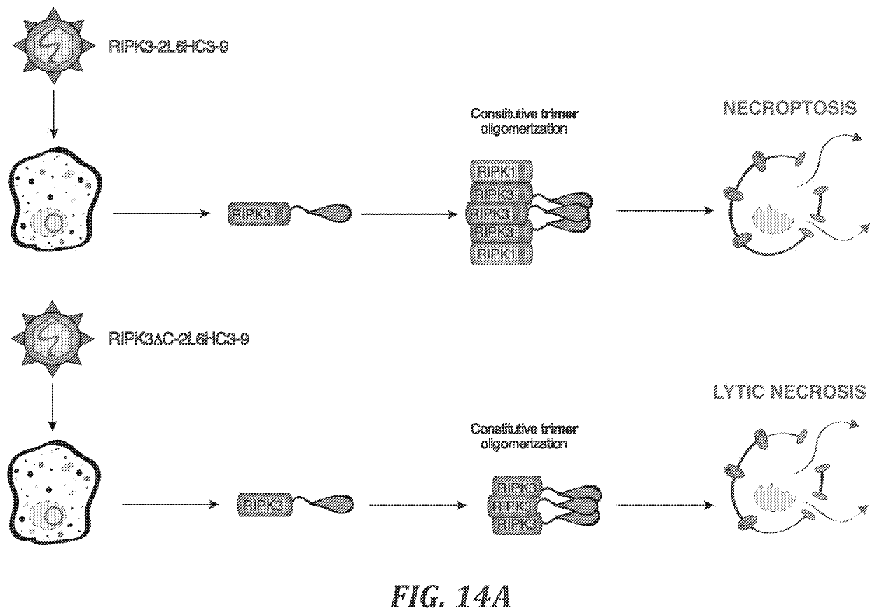

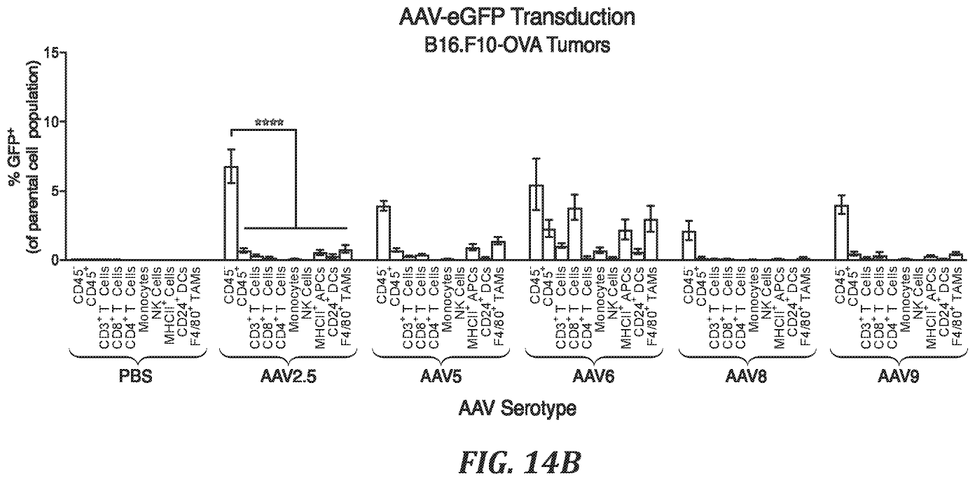

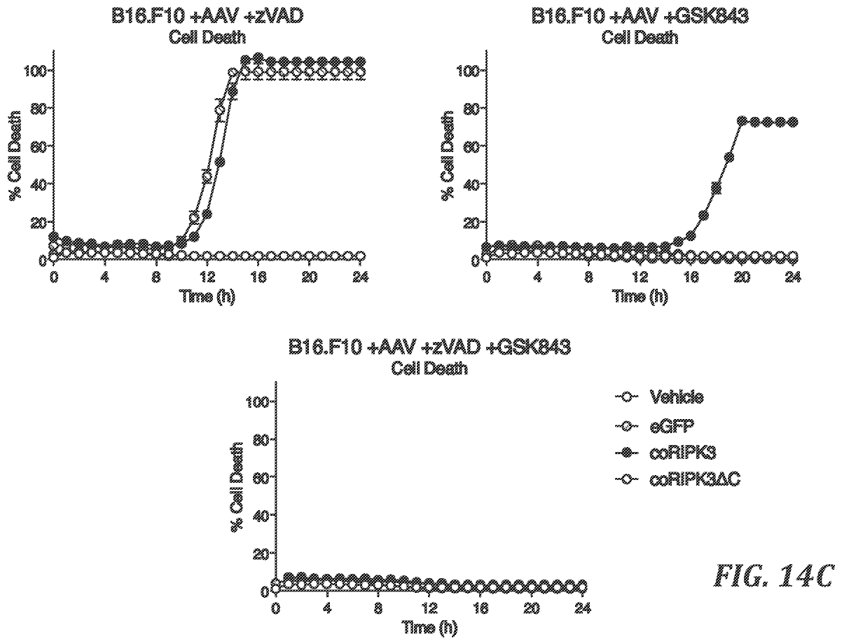

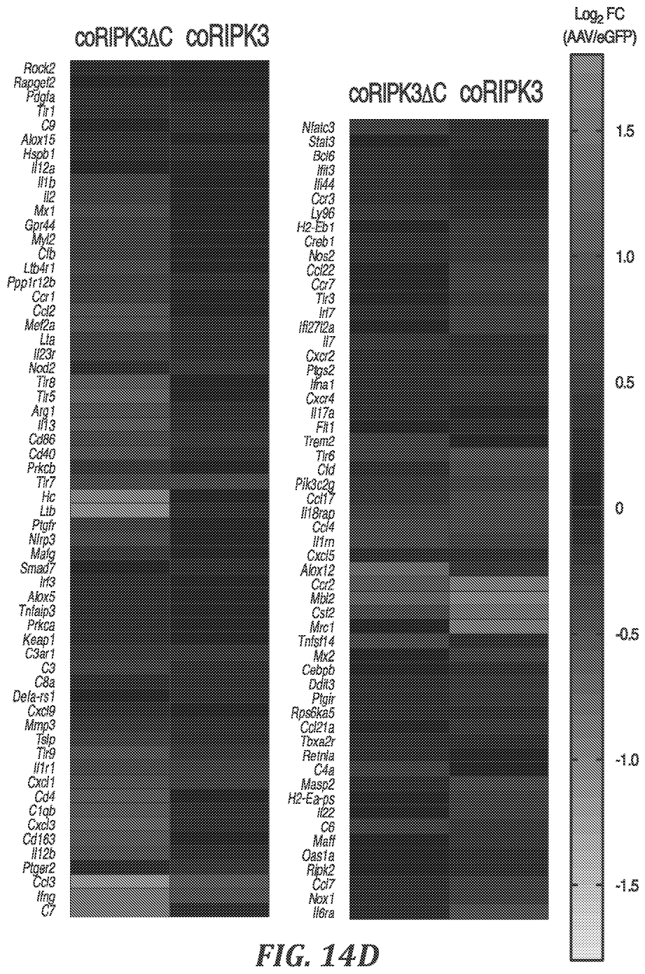

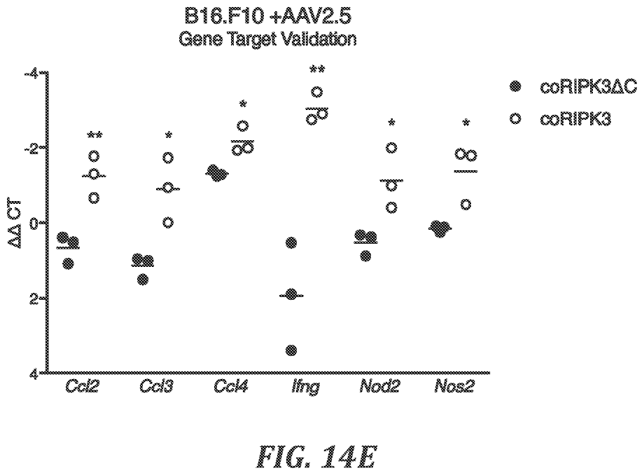

Intratumoral dying cell injection provides a cleanly controlled model for examining how exposure to stimuli derived from necroptotic cells can influence anti-tumor immune responses. However, an obvious caveat of this model is that it fails to assess immune responses to tumor cell necroptosis in situ. To address this, reagents were created that would allow direct induction of necroptosis in tumors in vivo. To achieve this, versions of RIPK3 fused to a constitutively-oligomerizing (“co”) domain were generated that contained high-affinity 2L6HC3-9 homotrimeric domains that have been previously synthesized and described (Boyken S. E., et al. (2016). De novo design of protein homo-oligomers with modular hydrogen-bond network-mediated specificity. Science. 6, 680-687, incorporated herein by reference in its entirety). These chimeric forms of RIPK3 undergo oligomerization and activation upon their expression in cells, independent of any upstream signaling or the presence of a ligand. To deliver these reagents to tumor cells, AAVs were created that contained genes encoding these constructs under control of a synthetic MND (myeloproliferative sarcoma virus enhancer, negative control region deleted, d1587rev primer-binding site substituted) promoter, enabling robust gene expression in target cells. Upon transduction of a target cell by these engineered AAVs, the fusion protein of interest is expressed, constitutively oligomerizes, and leads to rapid and specific induction of RIPK3-dependent cell death (FIG. 8A, FIG. 14A).

AAVs are a flexible tool for primary cell transduction, as several serotypes with varying cellular tropisms have been described. Therefore, an AAV serotype was sought that would selectively deliver construct expression to B16.F10 melanoma cells. Using a hybrid AAV2.5 serotype (Bowles D. E., et al. (2012). Phase 1 gene therapy for Duchenne muscular dystrophy using a translational optimized AAV vector. Mol Ther. 20, 443-455, incorporated herein by reference in its entirety), robust transduction of cultured B16.F10 tumor cells was observed within 24 hours of eGFP-AAV2.5 addition (FIG. 8B). Importantly, the AAV2.5 serotype also transduced non-leukocytic CD45⁻ cells within B16.F10-OVA tumors in vivo, exhibiting successful eGFP transduction in a higher percentage of CD45⁻ cells compared to AAV5, AAV6, AAV8, or AAV9 serotypes (FIG. 14B). eGFP-AAV2.5 also had the lowest percentage of off-target transduction of tumor-associated immune cells in vivo, across both myeloid and lymphoid cell populations (FIG. 14B). It was therefore concluded that the hybrid AAV2.5 serotype would maximize tumor cell transduction efficiency while limiting off-target transduction of immune cells when adapted for use in vivo.

Next, the kinetics of death induced by AAV2.5 particles that deliver genes encoding chimeric pro-death proteins in vitro were characterized. By imaging analysis, it was observed that transduction of B16.F10 tumor cells with necroptosis-targeting (coRIPK3) or lytic necrosis-targeting (coRIPK3ΔC) AAV2.5 leads to 100% cell death in tumor cells within ˜15 hours of AAV addition. Cells appeared to die via programmed necroptosis, as observed by cellular morphology observed under bright-field microscopy (data not shown) and via manipulation of cell death signaling using various death pathway inhibitors. The pan-caspase inhibitor zVAD-fmk did not affect death induction by coRIPK3- or coRIPK3ΔC-AAV2.5 (FIG. 14C, left panel). Addition of the RIPK3 inhibitor GSK-843 eliminated coRIPK3ΔC-AAV2.5-induced death while decreasing coRIPK3-AAV2.5-induced death, this latter effect was likely due to reverse signaling through the RIPK1/RIPK3 necrosome to induce apoptosis, as has been previously described (FIG. 14C, middle panel). Consistent with this, incubation with both zVAD-fmk and GSK-843 eliminated all cell death associated with coRIPK3-AAV2.5 treatment (FIG. 14, right panel). This set of experiments shows that either necroptosis or lytic necrosis can be specifically and rapidly induced in B16.F10 tumor cells in vitro upon transduction by coRIPK3- or coRIPK3ΔC-AAV2.5, respectively.

It was hypothesized that the immunostimulatory nature of necroptosis involved engagement of an inflammatory transcriptional response upon activation of RIPK3, but not RIPK3ΔC, as mutated RIPK3ΔC lack the ability of recruit RIPK1 and subsequent NF-κB-dependent gene transcription. To characterize transcriptional signatures associated with either coRIPK3-AAV2.5-induced necroptosis or coRIPK3ΔC-AAV2.5-induced lytic necrosis, we infected B16.F10 tumor cells in vitro with corresponding AAVs for 10 hours (a time point at which tumor cells have not yet undergone membrane permeabilization, allowing for nucleic acid isolation) and then harvested total RNA for Nanostring analysis. Using a pre-designed gene target panel to evaluate murine inflammatory gene expression, we found that transduction of tumor cells with coRIPK3-AAV2.5 yielded a distinct transcriptional signature compared to cells transduced with coRIPK3ΔC-AAV2.5 (FIG. S5D). Further examination of this signature revealed that necroptotic (coRIPK3) B16.F10 cells exhibited upregulated expression of numerous NF-κB-dependent gene targets, including Lta, Ltb, Cd40, Cd86, Mef2a Nod2, Nos2, and Creb1 in comparison to lytic necrotic (coRIPK3ΔC) tumor cells (FIG. 5D). Additionally, necroptotic B16.F10 cells also upregulated expression of several inflammatory chemokines and cytokines, including Cxcl1, Cxcl3, Ccl2, Ccl3, Ccl4, Ccl21a, Ccl22, Il12b, Il22, and Ifng (FIG. 8D). Upregulated transcript levels for several of these target genes were independently validated via qRT-PCR (FIG. 14E). Taken together, these data indicate that the induction of tumor cell death via coRIPK3-AAV2.5 transduction in vitro leads to an inflammatory transcriptional signature consistent with immunogenic necroptosis. Furthermore, this gene signature depends on the assembly of the RIPK1/RIPK3 necrosome via RHIM-RHIM interactions, as it is absent in tumor cells transduced with coRIPK3ΔC-AAV2.5.

Administration of Necroptosis-Targeting AAVs in Conjunction with α-PD-1 In Vivo Promotes Durable Tumor Clearance

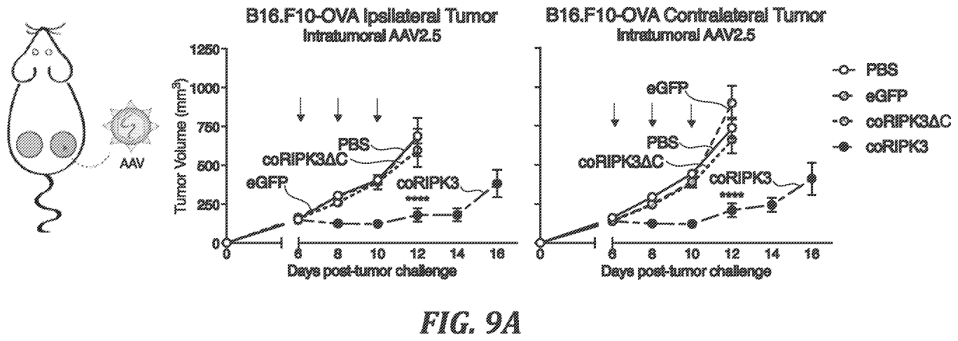

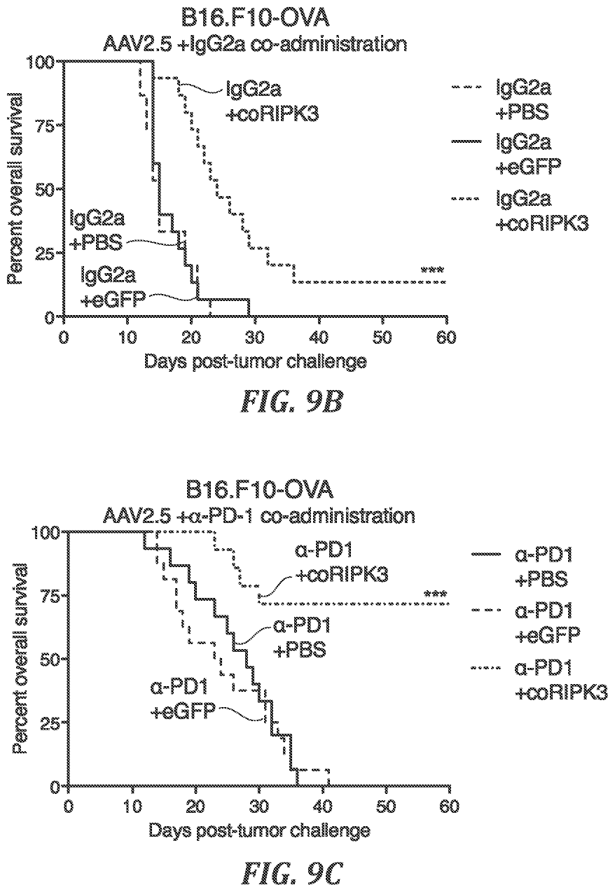

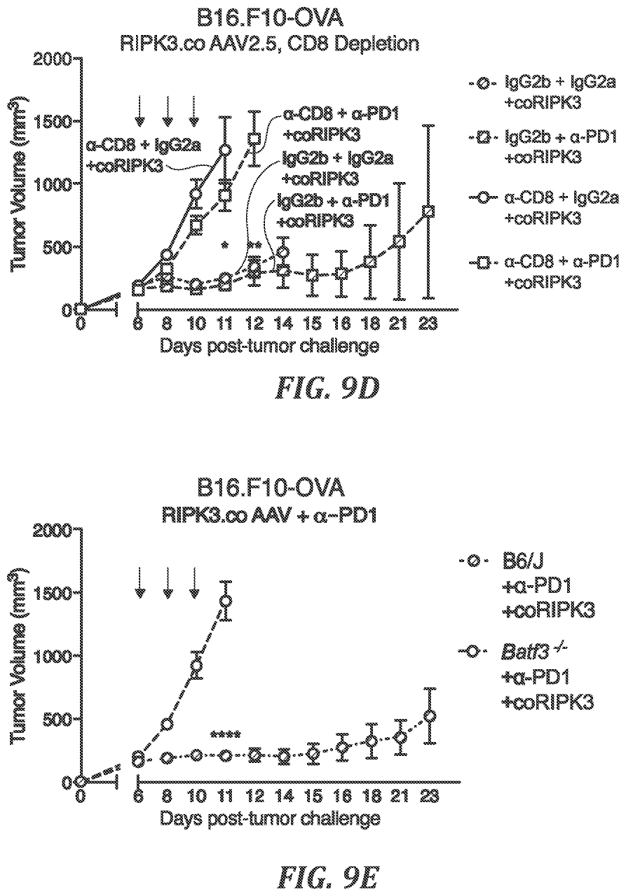

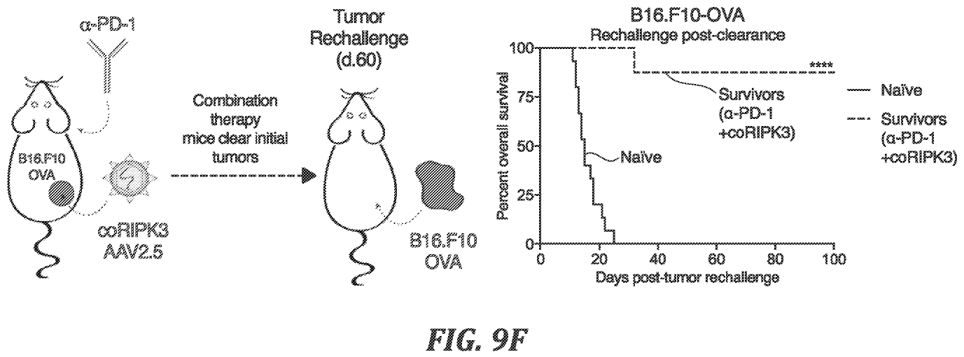

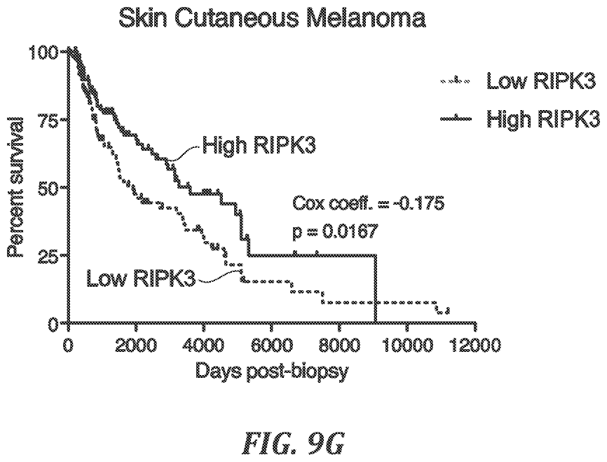

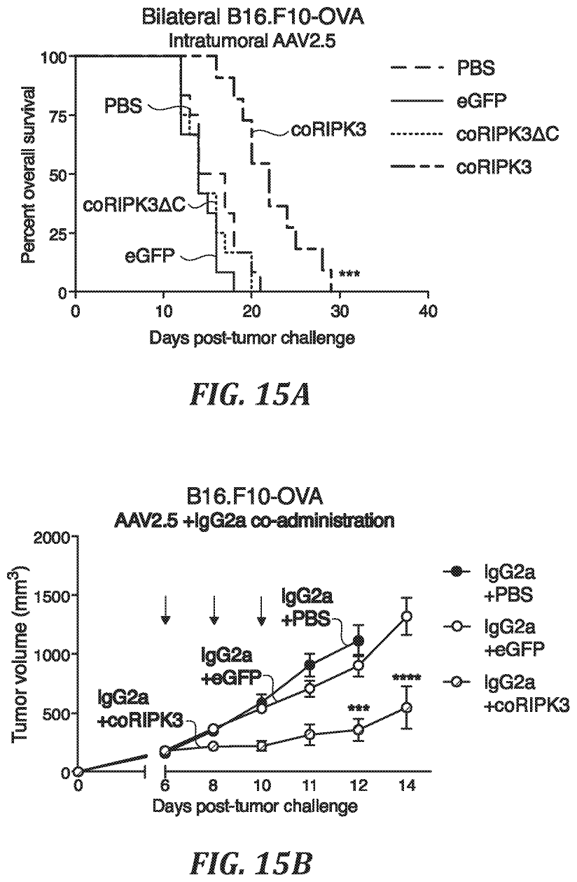

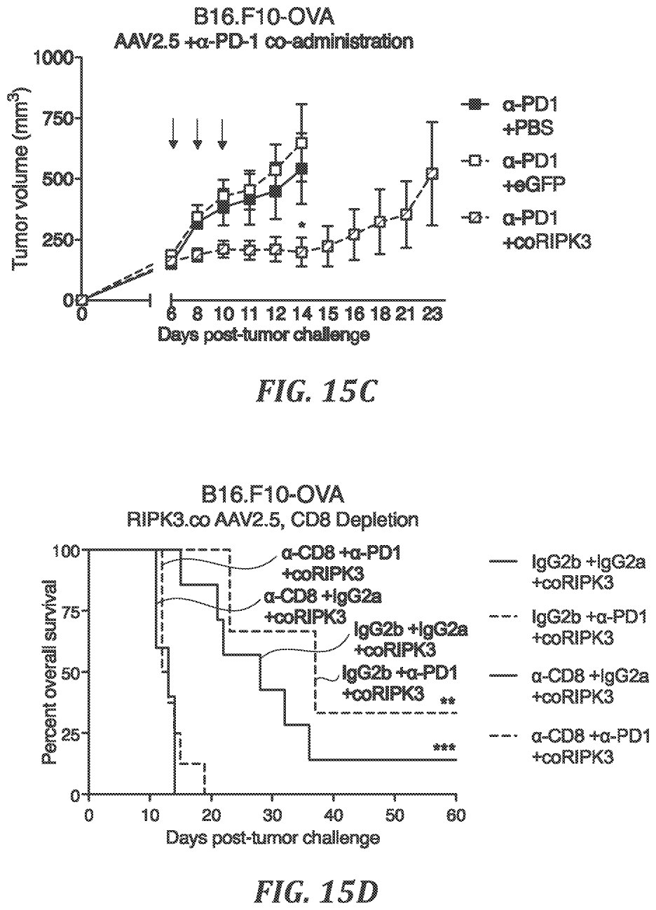

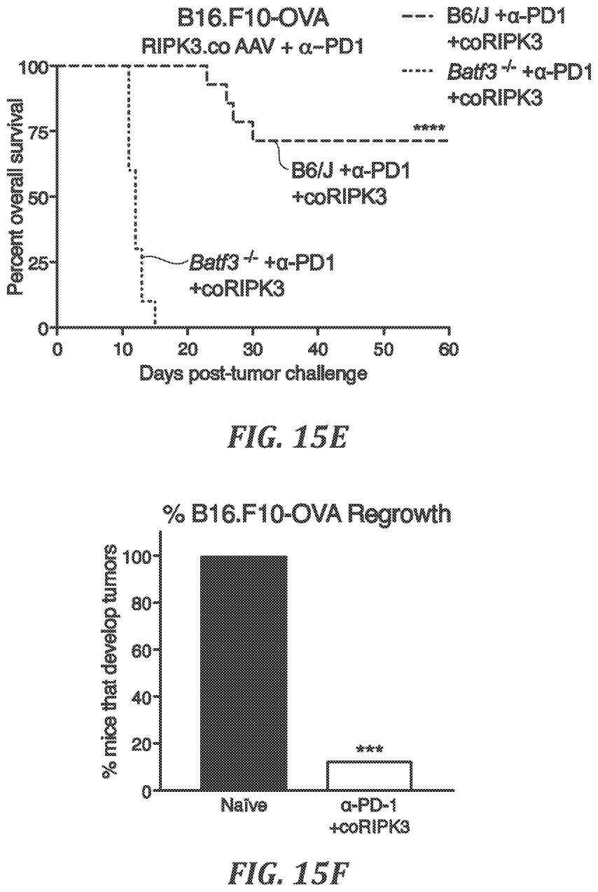

Following validation of the PCD-targeting AAVs in vitro, these tools were applied to study anti-tumor responses in vivo. Using a bilateral B16.F10-OVA flank tumor model, intratumoral administration of coRIPK3-AAV2.5 was found to confer control over tumor outgrowth in both treated (ipsilateral) and untreated (contralateral) tumors (FIG. 9A) and significantly extended animal survival (FIG. 15A) in comparison to intratumoral injection of coRIPK3ΔC-AAV2.5 or control eGFP-AAV2.5. These results recapitulated the tumor control effects that we observed in a bilateral tumor model using necroptotic fibroblast administration. Next, it was tested if necroptosis-targeting AAV could similarly protect mice from single B16.F10-OVA tumor outgrowth upon co-administration with the ICB agent α-PD-1. Not only did administration of coRIPK3-AAV2.5 with isotype controls significantly extend animal survival (FIG. 9B) and inhibit tumor growth (FIG. 15B) in comparison to eGFP-AAV2.5-treated control mice, but the co-administration of coRIPK3-AAV2.5 with α-PD-1 lead to robust responses, with complete tumor clearance in 69.2% of mice (FIG. 9C) and significant control over tumor outgrowth (FIG. 15C). Again, these tumor elimination responses closely paralleled those observed in the intratumoral necroptotic fibroblast injection model.

B16.F10-OVA tumor control following co-administration of coRIPK3-AAV2.5+isotype or α-PD-1 required the presence of CD8⁺ leukocytes, as depletion of CD8⁺ cell subsets via antibody injection completely abrogated the protective effects of coRIPK3-AAV2.5+IgG2a or α-PD-1 (FIG. 9D, FIG. 15D). Additionally, mice lacking Batf3⁺ cDC1 also failed to control B16.F10-OVA tumors following coRIPK3-AAV2.5+α-PD-1 treatment regimen (FIG. 9E, FIG. 15E). Considering that tumor control by necroptotic fibroblasts also necessitated the presence of these immune cell compartments, these experiments revealed similar effector cell subset requirements between both intratumoral dying fibroblast and intratumoral AAV models. Considering these requirements, it was tested if the mice that had successfully cleared their B16.F10-OVA tumors following dual therapy (FIG. 9C) had developed protective immune memory. To this end, surviving animals were re-challenged with identical tumor cells on the same flank that initially bore the B16.F10-OVA tumors (FIG. 9F, left panel). Strikingly, the majority of these animals were protected from mortality due to tumor outgrowth (FIG. 9F, right panel), as only 12.5% of mice regrew tumors (FIG. 15F) compared to 100% of naïve controls. Overall, these data demonstrate that intratumoral administration of necroptosis-targeting AAVs in conjunction with α-PD-1 confers durable, immune-mediated tumor rejection similar to that observed upon administration of intratumoral necroptotic NIH-3T3 fibroblasts.

AAV-mediated transduction of tumor cells allows for enforced expression of activated RIPK3, regardless of the expression status of endogenous RIPK3. Considering the beneficial effects of enforced RIPK3 activation that we observed in our murine melanoma model, we wanted to examine how endogenous levels of RIPK3 correlated with survival outcomes in human cancer patients. Using tumor biopsy RNAseq data available through The Cancer Genome Atlas (TCGA) database, we stratified human skin cutaneous melanoma patients based on upper (High) and lower quartiles (Low) of RIPK3 transcript expression within the tumor tissue. Strikingly, patients whose tumor biopsy samples fell within the bounds of high RIPK3 expression exhibited significantly improved survival outcomes compared to low RIPK-expressing patients (FIG. 9G). Furthermore, multivariate Cox regression modeling revealed a negative coefficient (−0.175), indicating that high expression of RIPK3 is correlated with a better survival outcome (FIG. 9G). Altogether, these results show that higher levels of RIPK3 expression within melanoma tumors is associated with improved survival in a subset of human patients.

Discussion

Distinct forms of PCD can differentially instruct subsequent immune responses mounted against antigens derived from dying cells. Here, a role for RIPK1/RIPK3-dependent necroptosis is described in which necroptotic fibroblasts within the TME drive increased antigen uptake and activation of tuAPCs to potentiate tumor-specific CD8⁺ T cell immunity, which synergizes with α-PD-1 co-administration to confer durable tumor rejection. Importantly, these gross tumor control effects are recapitulated in a model of AAV-mediated induction of necroptosis within melanoma tumor cells in situ, indicating that enforced activation of RIPK3 may lead to beneficial inflammatory signaling that is conserved across multiple cell types. Notably, existing therapies to target necroptosis in vivo exhibit variable efficacy due to off-target effects of global caspase inhibition and the differential status of pro-death signaling molecules in tumor cells. AAV-mediated reconstitution of constitutively active necroptotic signaling components within tumor cells represents a novel strategy to specifically induce necroptosis independently of any endogenous signaling requirements. Considering that high levels of RIPK3 expression in human melanoma tumors correlate with improved patient survival, such strategies to restore or increase necroptotic signaling in human tumors are a promising therapeutic target.

Across both dying cell and AAV administration models, the therapeutic effects of necroptotic cells appear to occur independently of intracellular DAMP release, as administration of lytic necrotic cells failed to recapitulate anti-tumor immune responses. This implies that transcriptional signaling downstream of the RIPK1/RIPK3 necrosome complex is required for therapeutic efficacy. This conclusion is further supported by the fact that singular deficiency of a variety of innate immune signaling molecules involved in cytosolic nucleic acid sensing, TLR signaling, or TNF-mediated inflammation in tumor-infiltrating leukocytes does not affect tumor control following immune stimulation via necroptosis. Although any of these pathways could provide a partial contribution to tumor control, none of the candidates tested were absolutely required for the therapeutic effect of necroptotic cells.

These results are consistent with reports of RIPK1- and NF-κB-dependent gene expression mediating the immunogenic effects of necroptotic cells with respect to dendritic cell maturation and priming of protective CD8⁺ T cells in vaccination models. A growing body of evidence has revealed death-independent functions of RIPK1/RIPK3 signaling that function independently of cell lysis via MLKL-mediated pore formation, including the production of protective inflammatory chemokines during neuroinvasive viral infection, or cytokines following TLR4 stimulation via LPS treatment or infection with avirulent strains of Gram-negative bacteria. Indeed, a RIPK1-NF-κB signaling axis is likely responsible for driving anti-tumor immune responses to necroptotic B16.F10 melanoma cells, as the Nanostring analysis of necroptotic tumor cells in vitro revealed upregulated expression of a variety of NF-κB-dependent gene targets, as well as several inflammatory chemokines and cytokines. Interestingly, the present observation of increased tumor-derived antigen within tuAPCs exposed to necroptotic cells is consistent with a previous report of necrotic debris being ingested alongside extracellular contents via macropinocytosis. However, the specific signals derived from necroptotic cells that are responsible for driving macropinocytosis to increase sampling of the local extracellular microenvironment remain unknown. Defining the mechanistic targets of RIPK1/RIPK3 activation and how these targets interact with tuAPCs to drive either increased macropinocytosis or improved retention of tumor antigen in order to better stimulate cytotoxic CD8⁺ T cells remains an important area for study.

Recent efforts have sought to test how stimulation of innate immune signaling pathways specifically within tumors can benefit anti-tumor responses. These strategies include agonism of nucleic acid sensing via cGAS/STING and TLR pathways, as well as inhibition of regulatory signals such as TAM RTKs on tumor-associated myeloid cells to eliminate repression of inflammatory responses. These studies highlight the therapeutic benefit of manipulating innate immune signaling targets within the tumor microenvironment to preferentially skew the polarization or activation of tuAPCs to more effectively promote tumor-reactive T cell responses. Here, it is demonstrated that necroptosis induction within the TME can similarly function to beneficially stimulate the activation of tuAPCs and subsequent CD8⁺ T cell-mediated immunity, which successfully synergizes with ICB to promote durable tumor rejection. The dynamic nature of tumor-immune interactions necessitates the identification of novel therapeutic targets to add to the existing arsenal of tumor immunotherapy. Tumor cell death represents a proximal event in the generation of tumor immunity, and specific modulation of PCD to maximize its immunogenicity may constitute an important orthogonal target to complement existing innate- and T cell-based forms of immunotherapy for optimal stimulation of anti-tumor immune responses.

Experimental Procedures

Cell Culture

B16.F10-OVA, LL/2-OVA, NIH-3T3, and HEK-293T cells were maintained in Dulbecco's modification of Eagle medium (DMEM) supplemented with 10% (vol/vol) FBS, 2 mM L-glutamine, 10 mM HEPES, and 1 mM sodium pyruvate (“complete DMEM”). E.G7-OVA cells were maintained in RPMI 1640 supplemented with 10% FBS, 2 mM L-glutamine, 10 mM HEPES, 1 mM sodium pyruvate, 0.05 mM beta-mercapthoethanol, 0.4 mg/mL geneticin (G418), and 4.5 g/L D-glucose. Bone marrow-derived macrophages (BMDMs) were cultured in complete DMEM+penicillin/streptomycin+20 ng/mL recombinant M-CSF and differentiated for 7 days prior to plating for experiments. All cells were cultured at 37° C. with 5% CO₂. B16.F10 and LL/2 cell lines transduced with a plasmid (pSLIK) encoding activatable versions of caspase-9 or RIPK3 under TRE control, so cells were cultured in 1 μg/mL doxycycline (Sigma) for 18 h to induce construct expression prior to harvesting for B/B homodimerizer pulse incubation as described below for dying cell injections.

Mice

C57BL6/J (B6/J) mice were purchased (Jackson Laboratories), while all other genotypes were bred and housed under specific-pathogen-free conditions at the University of Washington. All animals were housed at the University of Washington and maintained according to protocols approved by the University of Washington Institutional Animal Care and Use Committee (IACUC).

Tumor Models

6-10 week old female (B16.F10-OVA, E.G7-OVA) or male (LL/2-OVA) mice were injected subcutaneously on the right flank with 1×10⁵ (B16.F10-OVA, E.G7-OVA) or 2×10⁵ (LL/2-OVA) tumor cells, mixed in a 1:1 volumetric ratio with the basement membrane matrix Matrigel HC (Corning) for a final injection volume of 100 μL. For bilateral tumor experiments, mice were also implanted with syngeneic tumor cells on the left flank on the same day (d.0) of right flank tumor injection. Tumors were measured, and volume was calculated using the following formula as previously described (Corrales L., et al. (2015). Direct activation of STING in the tumor microenvironment leads to potent and systemic tumor regression and immunity. Cell Reports. 11, 1018-1030): Volume=Short axis²×Long axis×0.523. Mice were euthanized once tumor burden reached a volume≥2000 mm³. Mice that developed skin ulceration over the tumor site were euthanized and excluded from experimental analyses.

Intratumoral Dying Cell Injections

NIH-3T3, B16.F10, or LL/2 cells stably transduced with pro-death constructs were harvested, and 5×10⁶ cells/mL were incubated in complete DMEM+1 mM of B/B homodimerizer (Clontech) for 15 minutes at 37° C., mixed periodically to prevent cells from settling. Cells were then quenched with an excess of ice-cold 1× PBS, washed 3 times in cold PBS, counted, and resuspended at 20×10⁶ cells/mL in PBS and kept on ice prior to injection. 1×10⁶ dying cells were administered intratumorally in a 50 μL injection volume. Extra cells were re-plated and cultured at 37° C. overnight to ensure that <95% of treated cells underwent PCD for each set of injections. Dying cells were administered on days 6, 8, and 10 post-initial tumor challenge.

In Vivo Antibody Administration

For T cell depletion and immune checkpoint blockade experiments, 200 μg of α-CD8 (clone 2.43, BioXCell), α-CD4 (clone GK1.5, BioXCell), α-PD-1 (clone RMP1-14, BioXCell), or respective isotype controls were administered to mice via intraperitoneal injection on days 5, 7, 9, and 11 post-initial tumor challenge. NK cell depletion experiments followed the same dosing protocol, using 250 μg of α-NK1.1 (clone PK136, BioXCell). T cell and NK cell depletions were confirmed by staining peripheral blood for target cell populations on day 8 post-initial tumor challenge, as assessed via flow cytometry using antibody clones distinct from those targeted by depletion antibodies. T cells were gated on CD45⁺>CD19⁻ CD3⁺>either CD4⁺ or CD8⁺ cells, NK cells were gated on CD45⁺>CD19⁻ CD3⁻>NKp46⁺ DX5⁺ cells.

Recombinant AAV Cloning

Design and sequencing analysis of all plasmids was performed using Geneious software v.7.1 (Kearse M., et al. (2012). Geneious Basic: an integrated and extendable desktop software platform for the organization and analysis of sequence data. Bioinformatics. 28, 1647-1649). The 2L6HC3-13 trimer homo-oligomer domain was a gift from David Baker (Boyken S. E., et al. (2016). De novo design of protein homo-oligomers with modular hydrogen-bond network-mediated specificity. Science. 6, 680-687). Trimerizing RIPK3 constructs were directly cloned into a single-stranded AAV (ssAAV) vector using multi-fragment assembly (Infusion HD, Takara Biosciences). AAV backbone was linearized using SnaBI digest as previously described (Hubbard N., et al. (2016). Targeted gene editing restores regulated CD40L function in X-linked hyper-IgM syndrome. Blood. 127, 2513-2522). Primers for amplification of gene fragments were designed to contain 20 base pair 5′ and 3′ homology to neighboring fusion sequences, and PCR amplification was carried out using Q5 Polymerase (New England Biosciences). The shortened 3′ UTR WPRE and polyA elements were amplified from pAAV-CW3SL-EGFP, a gift from Bong-Kiun Kaang (Addgene plasmid #61463). Sense and anti-sense primer sequences were as follows:

Fragment 1 (MND Promoter):
(SEQ ID NO: 136)
(S) CGCCATGCTACTTATCTACGGAGTCGTGACCTAGGGAACAGAGAAA
CAGG,
(SEQ ID NO: 137)
(AS) TTCGAGGAAGTCAAAACAGCGTGG,
Fragment 2 (RIPK3 and RIPK3ΔC):
(SEQ ID NO: 138)
(S) CGCTGTTTTGACTTCCTCGAACCATGTCTTCTGTCAAGTTATGG,
(full length RIPK3 AS)
(SEQ ID NO: 139)
AGAACCACTCCCTTCTGATCCTTCGGAACCCGTACGCTTGTGGAAGGGCT
GCCAGC,
(RIPK3ΔC AS)
(SEQ ID NO: 140)
AGAACCACTCCCTTCTGATCCTTCGGAACCCGTACGTCATTGGATTCGGT
GGGGTC,
Fragment 3 (2L6HC3-13 homo-trimer domain):
(SEQ ID NO: 141)
(S) GATCAGAAGGGAGTGGTTCTCATATGGGTACGAAATACG,
(SEQ ID NO: 142)
(AS) CAGAGGTTGATTATGCGGCCTTAGTCACTTTTGGCGTTAATTT
TC,
Fragment 4 (sWPRE/polyA):
(SEQ ID NO: 143)
(S) GGCCGCATAATCAACCTCTGG,
(SEQ ID NO: 144)
(AS) CCGCCATGCTACTTATCTACAAAAAACCTCCCACATCTCCCCC.

The DNA sequence of inserted elements was verified by sequencing, and the integrity of the viral inverted terminal repeat (ITR) within the pAAV backbone confirmed by restriction digest using AhdI, BglI or SmaI, prior to viral production.

Adeno-Associated Virus (AAV) Production, Purification, and Quantification

AAVs were produced as described (Khan I. F., et al. (2011). AAV-mediated gene targeting methods for human cells. Nat Protocol. 6, 482-501, Sather B. D., et al. (2015). Efficient modification of CCRS in primary human hematopoietic cells using a megaTAL nuclease and AAV donor template. Sci Transl Med. 7, 307ra156). Briefly, AAV stocks were generated in HEK293T cells via PEI transfection using engineered death enzyme-encoding vector+serotype helper (pLTAAV). Transfected cells were harvested 48 hours post-transfection, lysed via freeze/thaw cycling, treated with 100 U/mL Universal Nuclease (Thermo) at 37° C. for 30 minutes, and purified via centrifugation over an iodixanol density step gradient. Titers of viral stocks were determined via fluorogenic quantitative reverse transcriptase PCR (qRT-PCR) analysis in conjunction with TaqMan reagents and a ViiA 7 Real-Time PCR apparatus (Applied Biosystems). qRT-PCR for viral titer used primers targeting the conserved ITR, using the following sequences:

(SEQ ID NO: 145)
(F) GGAACCCCTAGTGATGGAGTT,
(SEQ ID NO: 146)
(R) CGGCCTCAGTGAGCGA.

Intratumoral AAV Injections

1×10¹¹ infectious unit (IFU) of respective AAV were administered intratumorally in a 50 μL injection volume, diluted in 1× PBS as necessary. Virus aliquots used for in vivo experiments were thawed once following initial freezing post-purification. AAV injections were administered on days 6, 8, and 10 post-initial tumor challenge.

Flow Cytometry and Cell Sorting

Leukocytes were isolated from either tumor-adjacent inguinal lymph node (iLN) by mashing over a 70 μM strainer, or from tumor tissue by digesting minced tumors in 1× PBS+2.6 mg/mL Collagenase A (Sigma)+23 U/mL DNase I (Sigma) at 37° C. with agitation for 45 minutes prior to mashing tissue over a 70 μM strainer. 1-3×10⁶ cells were blocked with anti-CD16/32 (BD Biosciences) and stained with Zombie viability dye (BioLegend) in 1× PBS at room temperature for 30 min. Cells were then incubated with appropriate fluorochrome-conjugated antibodies in 1× PBS+0.5% FBS+2 mM EDTA at for 4° C. for 1 hour. Permeabilization and intranuclear staining were performed using a Foxp3 Intranuclear Transcription Factor Staining Kit (eBiosciences). Data were collected using an LSRII flow cytometer (BD) and analyzed using FlowJo software (Treestar). For sorting of zsGreen⁺ tuAPCs populations, B16.F10-OVA-zsGreen tumors were harvested 48 hours post-intratumoral dying cell injection, leukocytes were processed and stained as described above, and zsGreen⁺ tuAPC subsets were sorted using a FACSAria II (BD).

OT-I Proliferation Assay

Lymph nodes and spleens from OT-I TCR transgenic mice were processed and enriched for CD8⁺ OT-I T cells via negative selection using biotinylated antibodies against B220, CD4, CD11b, CD11c, and Ter119 (eBioscience). OT-I T cells were activated via 6 day co-culture with irradiated splenocytes pulsed with 100 ng/mL SL8 peptide (Invivogen). 20,000 previously-activated OT-I were labeled with 5 μM CellTrace Violet (Thermo Fisher) and plated with 4,000 sorted zsGreen⁺ tuAPC subsets in 96 well U-bottom plates for 48 hours prior to analysis of proliferation dye dilution via flow cytometry.

Serum Cytokine Assessment

Sera were harvested from mice receiving indicated intratumoral treatments 48 hours post-dying cell administration and stored for <2 weeks at −80° C. As a positive control for inflammatory mediators, B6/J mice were injected intraperitoneally with 40 mg/kg of the STING agonist DMXAA (ApexBio), and sera were harvested 5 hours post-injection. Thawed serum samples were analyzed using a Th1/Th2 ProcartaPlex™ Panel 1 Luminex kit (Thermo Fisher) to quantify serum cytokines and chemokines.

Nanostring RNA Analysis and qRT-PCR

2×10⁶ B16.F10 cells were infected with 1×10¹¹ IFU of respective AAV for 10 hours. Total RNA was isolated using a Nucleospin RNA Kit (Macherey-Nagel) and run on an nCounter Sprint in conjunction with an nCounter Mouse Inflammation V2 Panel (Nanostring). Data were normalized and analyzed using nSolver software (Nanostring). For target gene validation, oligo(dT) random hexamers and SuperScript III Reverse Transcriptase (Life Technologies) were used to synthesize cDNA from the same total RNA samples used for Nanostring analysis. Fluorogenic quantitative reverse transcriptase PCR (qRT-PCR) analysis was performed using previously published oligonucleotide primer sequences using SYBR Green reagents and a ViiA 7 Real-Time PCR apparatus (Applied Biosystems). Cycle threshold (CT) values for target genes were normalized to CT values of the housekeeping gene Gapdh (ΔCT=CT_{Target}−CT_{Gapdh}), and subsequently normalized to baseline control values (ΔΔCT=ΔCT_{Experimental}−ΔCT_{Control}).

In Vitro Cell Death Assay

1×10⁵ B16.F10-OVA cells were cultured in 500 μL complete DMEM+1×10¹¹ IFU of respective AAV in 24 well plates for 24 hours. Cell viability was evaluated via incorporation of cell viability dye Sytox Green (Molecular Probes) or Yoyo-3 (200 nM, Life Technologies) and quantified using a 2-color Incucyte Zoom bioimaging platform (Essen Biosciences), as described (Brault et al., 2017). Where indicated, 50 μM zVAD-fmk (SM Biochemicals) or 100 nM GSK-873 (GlaxoSmithKline) were added to inhibit pan-caspase activation or RIPK3 activation, respectively.

In Vitro Dextran Uptake Assay

Differentiated BMDMs were plated in a 1:5 ratio with either live or RIPK3-expressing B16.F10-zsGreen tumor cells. Necroptotic tumor cells were induced to die upon 18 h incubation with 1 μg/mL doxycycline prior to plating in co-culture with BMDMs, then cultured with 100 nM BB homodimerizer (Clontech) for 24 h before adding 1 mg/mL Dextran-PE.TexasRed (10,000 MW, Thermo Scientific). Dextran incubations were performed in triplicate at either 4° C. or 37° C. for 30 min, and plates were tapped lightly every 10 minutes to mix. Cells were washed 3× in cold FACS buffer, stained with fluorochrome-conjugated antibodies, and immediately analyzed on a flow cytometer. Dextran uptake was calculated as follows: ΔgMFI=(gMFI dextran binding at 37° C.−gMFI dextran binding at 4° C.).

TCGA Analysis

The OncoLnc package (Anaya J. (2016). OncoLnc: linking TCGA survival data to mRNAs, miRNAs, and lncRNAs. PeerJ Comp Sci. 2, e67) was used to analyze RNASeqV2 and overall survival data generated by The Cancer Genome Atlas Research Network database (Weinstein J. N., et al. (2013). The Cancer Genome Atlas pan-cancer analysis project. Nat Genet. 45, 1113-1120). OncoLnc was used to conduct survival analyses using multivariate Cox regression modeling, assign log rank p values and Cox coefficients to assess significance, and generate Kaplan-Meier survival curves.

Statistics

Survival curves were analyzed via Mantel-Cox log-rank test unless noted otherwise. All other experiments were compared using parametric 2-tailed student's t-test, or 1-way or 2-way ANOVA, with appropriate corrections for repeated measures of tumor growth curves. All statistical analyses were performed using GraphPad Prism software unless noted otherwise.

While illustrative embodiments have been illustrated and described, it will be appreciated that various changes can be made therein without departing from the spirit and scope of the invention.

1. A fusion protein comprising a death inducing domain and a multimerization domain.

2. (canceled)

3. The fusion protein of claim 1, wherein the death inducing domain induces programmed cell death when the fusion protein forms a complex with a protein comprising one or more further death inducing domains.

4. (canceled)

5. The fusion protein of claim 1, wherein the death inducing domain comprises an effector domain selected from:
a caspase effector domain,
a kinase effector domain,
a MLKL effector domain, and
a gasdermin effector domain.

6. The fusion protein of claim 5, wherein the death inducing domain comprises a caspase effector domain selected from:
a caspase 1 effector domain,
a caspase 2 effector domain,
a caspase 3 effector domain
a caspase 4 effector domain,
a caspase 5 effector domain,
a caspase 8 effector domain,
a caspase 9 effector domain,
a caspase 10 effector domain, and
a caspase 11 effector domain.

7. (canceled)

8. The fusion protein of claim 5, wherein the death inducing domain comprises a kinase effector domain selected from:
a RIPK1 kinase effector domain,
a RIPK2 kinase effector domain, and
a RIPK3 kinase effector domain.

9. (canceled)

10. The fusion protein of claim 5, wherein the death inducing domain comprises an MLKL effector domain and further comprises a pseudokinase domain.

11. (canceled)

12. The fusion protein of claim 5, wherein the death inducing domain comprises a gasdermin effector domain, and further comprises an auto-inhibitory domain.

13-15. (canceled)

16. The fusion protein of claim 1, wherein the multimerization domain comprises a domain selected from:
2LHC2-23,
5L6HC3-1,
2L6HC3-9,
2L6HC3-13,
6H8, and
7H3.

17. The fusion protein of claim 1, wherein the death inducing domain and the multimerization domain are joined by a linker domain.

18-20. (canceled)

21. A nucleic acid comprising a sequence encoding the fusion protein of claim 1.

22. (canceled)

23. A vector comprising the nucleic acid of claim 21 and a promoter sequence operatively linked to the sequence encoding the fusion protein.

24-25. (canceled)

26. A cell comprising the nucleic acid of claim 21.

27. (canceled)

28. A method of inducing programmed cell death in a cell, comprising providing the cell with multiple copies of the fusion protein of claim 1.

29. The method of claim 28, wherein providing the cell with multiple copies of the fusion protein comprises contacting the cell with nucleic acid comprising a sequence encoding the fusion protein or a vector comprising the nucleic acid and a promoter sequence operatively linked to the sequence under conditions that permit expression of the fusion protein in the cell.

30-34. (canceled)

35. The method of claim 28, wherein the cell is a tumor cell, a tumor-associated stromal cell, or a fibroblast.

36-37. (canceled)

38. A method of inhibiting tumor cell growth in a subject, comprising administering to the subject an effective amount of:
the nucleic acid of claim 21,
a vector comprising the nucleic acid of claim 21 and a promoter sequence operatively linked to the sequence encoding the fusion protein, or
a cell comprising the fusion protein encoded by the nucleic acid of claim 21.

39. (canceled)

40. The method of claim 38, wherein the effective amount of the nucleic acid, vector, or cell is administered intratumorally.

41. The method of claim 38, further comprising administering to the subject an effective amount of an immunotherapeutic agent.

42-44. (canceled)

45. A method of inhibiting growth of tumor cells in a subject, comprising modifying the tumor microenvironment of the tumor cells to contain necroptotic cells, wherein the necroptotic cells comprise multimers of an engineered protein comprising a death inducing domain that induces necroptosis.

46. (canceled)

47. The method of claim 45, wherein the engineered protein is a fusion protein comprising a death inducing domain and a multimerization domain.

48-49. (canceled)

50. The method of claim 45, wherein modifying the tumor microenvironment comprises:
generating the necroptotic cells in vitro by expressing the engineered protein in cells under conditions that permit multimerization of the engineered protein, and
administering the necroptotic cells to the tumor microenvironment prior to the death of at least some of the necroptotic cells.

51-52. (canceled)

53. The method of claim 50, wherein the cells are autologous with respect to the tumor cells in the tumor microenvironment.

54-67. (canceled)

68. The method of claim 38, wherein the other cell is a tumor cell, a tumor-associated stromal cell, or a fibroblast.

69. (canceled)
